(19) United States
(12) Patent Application Publication
(10) Pub. No.: US 2026/0162760 A1
(43) Pub. Date: Jun. 11, 2026

(54) TECHNIQUES FOR PREDICTING IMMUNE-RELATED ADVERSE EVENTS

(71) Applicant: BostonGene Corporation, Waltham, MA (US)

(72) Inventors: Aleksandr Zaitsev, Yerevan (AM); Evgenii Bolshakov, Moscow (RU); Maria Savchenko, Yerevan (AM); Sofya Kust, Boston, MA (US); Michael F. Goldberg, Boston, MA (US); Ravshan Ataullakhanov, Moscow (RU); Tatiana Vasileva, Yerevan (AM); Anastasiia Terenteva, Yerevan (AM); Aleksandra Brunovlenskaia-Bogoiavlenskaia, Moscow (RU); Alena Frank, Yerevan (AM); Anastasiia Bolshakova, Yerevan (AM); Ani Aloyan, Yerevan (AM); Svetlana Bezlepkina, Yerevan (AM); Anna Vardazaryan, Yerevan (AM); Nazar Arutiunian, Yerevan (AM); Dmitrii Fastovets, Yerevan (AM)

(21) Appl. No.: 19/378,273

(22) Filed: Nov. 3, 2025

Related U.S. Application Data
(60) Provisional application No. 63/715,796, filed on Nov. 4, 2024.

(51) Int. Cl. G16B 5/00; G16B 30/10; G16B 40/20; G16H 50/30

(52) U.S. Cl. CPC G16B 5/00; G16B 30/10; G16B 40/20; G16H 50/30

(57) ABSTRACT

Described herein are techniques for predicting whether a subject will experience an immune-related adverse event (irAE) in response to administration of an immune checkpoint inhibitor (ICI) therapy. In some embodiments, the techniques include: determining a likelihood that the subject will experience the irAE in response to administration of the ICI therapy, the determining comprising: performing: (a) processing clinical data using a first machine learning (ML) model to output a first likelihood that the subject will experience the irAE, (b) processing RNA sequencing data using a second ML model to output a second likelihood that the subject will experience the irAE, and/or (c) processing immune receptor data using a third ML model to output a third likelihood that the subject will experience the irAE; and processing the first, second, and/or third likelihoods using a fourth ML model trained to predict the likelihood that the subject will experience the irAE.

### CROSS-REFERENCE TO RELATED APPLICATIONS

This application claims the benefit of and priority under 35 U.S.C. § 119(e) of the filing date of U.S. Provisional Application No. 63/715,796, filed Nov. 4, 2024, and entitled “TECHNIQUES FOR PREDICTING IMMUNE-RELATED ADVERSE EVENTS,” which is incorporated by reference herein in its entirety.

### BACKGROUND

Immune checkpoint blockade targeting regulatory molecules (e.g., immune checkpoint inhibitors) are used for treating solid tumors, showing efficacy in multiple cancers including, for example melanoma, non-small cell lung carcinoma, and esophageal cancers. Examples of immune checkpoint inhibitors include anti-PD-1, anti-PD-L1, and CTLA-4. However, immune checkpoint blockage targeting regulatory molecules can lead to immune-related adverse events (irAE) of varying degrees of severity that may cause early treatment discontinuation, negative side effects, and, in some cases, death.

### SUMMARY

Some aspects provide for a method for predicting whether a subject will experience an immune-related adverse event (irAE) in response to administration of an immune checkpoint inhibitor (ICI) therapy to the subject, the method comprising: using at least one processor to perform: obtaining healthcare data for the subject, the healthcare data comprising at least two of: clinical data for the subject, RNA sequencing data for the subject, and immune receptor data for the subject; determining, using at least some of the healthcare data, a likelihood that the subject will experience the irAE in response to administration of the ICI therapy, the determining comprising: performing at least two of: (a) processing the clinical data for the subject using a first machine learning (ML) model to output a first likelihood that the subject will experience the irAE in response to administration of the ICI therapy, wherein the first ML model is trained to predict, from clinical data for a particular subject, a likelihood that the particular subject will experience the irAE in response to administration of the ICI therapy, (b) processing the RNA sequencing data for the subject using a second ML model to output a second likelihood that the subject will experience the irAE in response to administration of the ICI therapy, wherein the second ML model is trained to predict, from RNA sequencing data for a particular subject, a likelihood that the particular subject will experience the irAE in response to administration of the ICI therapy, and (c) processing the immune receptor data for the subject using a third ML model to output a third likelihood that the subject will experience the irAE in response to administration of the ICI therapy, wherein the third ML model is trained to predict, from immune receptor data for a particular subject, a likelihood that the particular subject will experience the irAE in response to administration of the ICI therapy; and processing two or more of the first, second, and third likelihoods using a fourth ML model trained to predict the likelihood that the subject will experience the irAE in response to administration of the ICI therapy from one or more of the likelihoods that the subject will experience the irAE determined using two or more of first-, second- and third-ML models; and outputting the likelihood that the subject will experience the irAE in response to administration of the ICI therapy.

Some aspects provide for a system, comprising: at least one processor; and at least one non-transitory computer-readable storage medium storing processor-executable instructions that, when executed by the at least one processor, cause the at least one processor to perform a method for predicting whether a subject will experience an immune-related adverse event (irAE) in response to administration of an immune checkpoint inhibitor (ICI) therapy to the subject, the method comprising: obtaining healthcare data for the subject, the healthcare data comprising at least two of: clinical data for the subject, RNA sequencing data for the subject, and immune receptor data for the subject; determining, using at least some of the healthcare data, a likelihood that the subject will experience the irAE in response to administration of the ICI therapy, the determining comprising: performing at least two of: (a) processing the clinical data for the subject using a first machine learning (ML) model to output a first likelihood that the subject will experience the irAE in response to administration of the ICI therapy, wherein the first ML model is trained to predict, from clinical data for a particular subject, a likelihood that the particular subject will experience the irAE in response to administration of the ICI therapy, (b) processing the RNA sequencing data for the subject using a second ML model to output a second likelihood that the subject will experience the irAE in response to administration of the ICI therapy, wherein the second ML model is trained to predict, from RNA sequencing data for a particular subject, a likelihood that the particular subject will experience the irAE in response to administration of the ICI therapy, and (c) processing the immune receptor data for the subject using a third ML model to output a third likelihood that the subject will experience the irAE in response to administration of the ICI therapy, wherein the third ML model is trained to predict, from immune receptor data for a particular subject, a likelihood that the particular subject will experience the irAE in response to administration of the ICI therapy; and processing two or more of the first, second, and third likelihoods using a fourth ML model trained to predict the likelihood that the subject will experience the irAE in response to administration of the ICI therapy from one or more of the likelihoods that the subject will experience the irAE determined using two or more of first-, second- and third-ML models; and outputting the likelihood that the subject will experience the irAE in response to administration of the ICI therapy.

Some aspects provide for a least one non-transitory computer-readable storage medium storing processor-executable instructions that, when executed by at least one processor, cause the at least one processor to perform a method for predicting whether a subject will experience an immune-related adverse event (irAE) in response to administration of an immune checkpoint inhibitor (ICI) therapy to the subject, the method comprising: obtaining healthcare data for the subject, the healthcare data comprising at least two of: clinical data for the subject, RNA sequencing data for the subject, and immune receptor data for the subject; determining, using at least some of the healthcare data, a likelihood that the subject will experience the irAE in response to administration of the ICI therapy, the determining comprising: performing at least two of: (a) processing the clinical data for the subject using a first machine learning (ML) model to output a first likelihood that the subject will experience the irAE in response to administration of the ICI therapy, wherein the first ML model is trained to predict, from clinical data for a particular subject, a likelihood that the particular subject will experience the irAE in response to administration of the ICI therapy, (b) processing the RNA sequencing data for the subject using a second ML model to output a second likelihood that the subject will experience the irAE in response to administration of the ICI therapy, wherein the second ML model is trained to predict, from RNA sequencing data for a particular subject, a likelihood that the particular subject will experience the irAE in response to administration of the ICI therapy, and (c) processing the immune receptor data for the subject using a third ML model to output a third likelihood that the subject will experience the irAE in response to administration of the ICI therapy, wherein the third ML model is trained to predict, from immune receptor data for a particular subject, a likelihood that the particular subject will experience the irAE in response to administration of the ICI therapy; and processing two or more of the first, second, and third likelihoods using a fourth ML model trained to predict the likelihood that the subject will experience the irAE in response to administration of the ICI therapy from one or more of the likelihoods that the subject will experience the irAE determined using two or more of first-, second- and third-ML models; and outputting the likelihood that the subject will experience the irAE in response to administration of the ICI therapy.

Some aspects provide for a method for predicting whether a subject will experience an immune-related adverse event (irAE) in response to administration of an immune checkpoint inhibitor (ICI) therapy to the subject, the method comprising: using at least one processor to perform: determining, using RNA sequencing data and/or immune cell data, (i) a proportion of classical dendritic cells (cDCs) to dendritic cells and (ii) a proportion of memory T cells to T cells; determining a plurality of immune signatures using the RNA sequencing data, each of the plurality of immune signatures representing RNA expression levels for at least some genes in a respective gene group of a plurality of gene groups; processing (i) the proportion of cDCs to dendritic cells, (ii) the proportion of memory T cells to T cells, and (iii) the plurality of immune signatures, using a machine learning (ML) model trained to predict a likelihood that the subject will experience the irAE in response to administration of the ICI therapy from (i) the proportion of cDCs to dendritic cells, (ii) the proportion of memory T cells to T cells, and (iii) the plurality of immune signatures; and outputting the likelihood that the subject will experience the irAE in response to administration of the ICI therapy.

Some aspects provide for a system, comprising: at least one processor; and at least one non-transitory computer-readable storage medium storing processor-executable instructions that, when executed by the at least one processor, cause the at least one processor to perform a method for predicting whether a subject will experience an immune-related adverse event (irAE) in response to administration of an immune checkpoint inhibitor (ICI) therapy to the subject, the method comprising: determining, using RNA sequencing data and/or immune cell data, (i) a proportion of classical dendritic cells (cDCs) to dendritic cells and (ii) a proportion of memory T cells to T cells; determining a plurality of immune signatures using the RNA sequencing data, each of the plurality of immune signatures representing RNA expression levels for at least some genes in a respective gene group of a plurality of gene groups; processing (i) the proportion of cDCs to dendritic cells, (ii) the proportion of memory T cells to T cells, and (iii) the plurality of immune signatures, using a machine learning (ML) model trained to predict a likelihood that the subject will experience the irAE in response to administration of the ICI therapy from (i) the proportion of cDCs to dendritic cells, (ii) the proportion of memory T cells to T cells, and (iii) the plurality of immune signatures; and outputting the likelihood that the subject will experience the irAE in response to administration of the ICI therapy.

Some aspects provide for at least one non-transitory computer-readable storage medium storing processor-executable instructions that, when executed by at least one processor, cause the at least one processor to perform a method for predicting whether a subject will experience an immune-related adverse event (irAE) in response to administration of an immune checkpoint inhibitor (ICI) therapy to the subject, the method comprising: determining, using RNA sequencing data and/or immune cell data, (i) a proportion of classical dendritic cells (cDCs) to dendritic cells and (ii) a proportion of memory T cells to T cells; determining a plurality of immune signatures using the RNA sequencing data, each of the plurality of immune signatures representing RNA expression levels for at least some genes in a respective gene group of a plurality of gene groups; processing (i) the proportion of cDCs to dendritic cells, (ii) the proportion of memory T cells to T cells, and (iii) the plurality of immune signatures, using a machine learning (ML) model trained to predict a likelihood that the subject will experience the irAE in response to administration of the ICI therapy from (i) the proportion of cDCs to dendritic cells, (ii) the proportion of memory T cells to T cells, and (iii) the plurality of immune signatures; and outputting the likelihood that the subject will experience the irAE in response to administration of the ICI therapy.

Some aspects provide for a method for predicting whether a subject will develop inflammatory bowel disease (IBD) in response to administration of an immune checkpoint inhibitor (ICI) therapy, the method comprising: using at least one processor to perform: obtaining sequencing data for the subject, the sequencing data indicating whether a plurality of human leukocyte antigen (HLA) alleles are present in a genome of the subject, the plurality of HLA alleles comprising: (i) a first set of HLA alleles associated with a risk of IBD, and (ii) a second set of HLA alleles not associated with a risk of IBD; providing, as input to a machine learning (ML) model, a plurality of input features including: (i) a first input feature indicative of a number of HLA alleles in the first set of HLA alleles associated with the risk of IBD that are present in the genome of the subject, (ii) a second input feature indicative of a number of HLA alleles in the second set of HLA alleles not associated with the risk of IBD that are present in the genome of the subject, and (iii) one or more third input features indicative of HLA alleles present in the genome of the subject; processing the input using the ML model to output a likelihood that the subject will develop IBD in response to the administration of the ICI therapy, wherein the ML model is trained to predict the likelihood that the subject will develop IBD in response to administration of the ICI therapy from (i) the first input feature, (ii) the second input feature, and (iii) the one or more third input features; and outputting the likelihood that the subject will develop IBD in response to the administration of the ICI therapy.

Some aspects provide for a system, comprising: at least one processor; and at least one non-transitory computer-readable storage medium storing processor-executable instructions that, when executed by the at least one processor, cause the at least one processor to perform a method for predicting whether a subject will develop inflammatory bowel disease (IBD) in response to administration of an immune checkpoint inhibitor (ICI) therapy, the method comprising: obtaining sequencing data for the subject, the sequencing data indicating whether a plurality of human leukocyte antigen (HLA) alleles are present in a genome of the subject, the plurality of HLA alleles comprising: (i) a first set of HLA alleles associated with a risk of IBD, and (ii) a second set of HLA alleles not associated with a risk of IBD; providing, as input to a machine learning (ML) model, a plurality of input features including: (i) a first input feature indicative of a number of HLA alleles in the first set of HLA alleles associated with the risk of IBD that are present in the genome of the subject, (ii) a second input feature indicative of a number of HLA alleles in the second set of HLA alleles not associated with the risk of IBD that are present in the genome of the subject, and (iii) one or more third input features indicative of HLA alleles present in the genome of the subject; processing the input using the ML model to output a likelihood that the subject will develop IBD in response to the administration of the ICI therapy, wherein the ML model is trained to predict the likelihood that the subject will develop IBD in response to administration of the ICI therapy from (i) the first input feature, (ii) the second input feature, and (iii) the one or more third input features; and outputting the likelihood that the subject will develop IBD in response to the administration of the ICI therapy.

Some aspects provide for at least one non-transitory computer-readable storage medium storing processor-executable instructions that, when executed by at least one processor, cause the at least one processor to perform a method for predicting whether a subject will develop inflammatory bowel disease (IBD) in response to administration of an immune checkpoint inhibitor (ICI) therapy, the method comprising: obtaining sequencing data for the subject, the sequencing data indicating whether a plurality of human leukocyte antigen (HLA) alleles are present in a genome of the subject, the plurality of HLA alleles comprising: (i) a first set of HLA alleles associated with a risk of IBD, and (ii) a second set of HLA alleles not associated with a risk of IBD; providing, as input to a machine learning (ML) model, a plurality of input features including: (i) a first input feature indicative of a number of HLA alleles in the first set of HLA alleles associated with the risk of IBD that are present in the genome of the subject, (ii) a second input feature indicative of a number of HLA alleles in the second set of HLA alleles not associated with the risk of IBD that are present in the genome of the subject, and (iii) one or more third input features indicative of HLA alleles present in the genome of the subject; processing the input using the ML model to output a likelihood that the subject will develop IBD in response to the administration of the ICI therapy, wherein the ML model is trained to predict the likelihood that the subject will develop IBD in response to administration of the ICI therapy from (i) the first input feature, (ii) the second input feature, and (iii) the one or more third input features; and outputting the likelihood that the subject will develop IBD in response to the administration of the ICI therapy.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a diagram of an illustrative technique for predicting whether a subject will experience an immune-related adverse event (irAE) in response to administration of an immune checkpoint inhibitor (ICI) therapy to the subject, according to some embodiments of the technology described herein.

FIG. 1B is a diagram of an illustrative technique for determining a likelihood that a subject will experience an irAE in response to administration of an ICI therapy, according to some embodiments of the technology described herein.

FIG. 1C is a diagram of an illustrative technique for determining, from clinical data for a subject, the likelihood that a subject will experience an irAE in response to administration of an ICI therapy, according to some embodiments of the technology described herein.

FIG. 1D is a diagram of an illustrative technique for determining, from sequencing data and (optionally) immune cell data for a subject, the likelihood that a subject will experience an irAE in response to administration of an ICI therapy, according to some embodiments of the technology described herein.

FIG. 1E is a diagram of an illustrative technique for determining, from immune receptor data for a subject, the likelihood that a subject will experience an irAE in response to administration of an ICI therapy, according to some embodiments of the technology described herein.

FIG. 1F is a diagram of an illustrative technique for predicting whether a subject will develop inflammatory bowel disease (IBD) in response to administration of an ICI therapy, according to some embodiments of the technology described herein.

FIG. 2 is a block diagram of an example system for predicting whether a subject will experience an irAE in response to administration of an ICI therapy to the subject, according to some embodiments of the technology described herein.

FIG. 3A is a flowchart of an illustrative process for predicting, from healthcare data for a subject, whether the subject will experience an irAE in response to administration of an ICI therapy, according to some embodiments of the technology described herein.

FIG. 3B is a flowchart of an illustrative process for predicting, from sequencing data and/or immune cell data for a subject, whether the subject will experience an irAE in response to administration of an ICI therapy, according to some embodiments of the technology described herein.

FIG. 3C is a flowchart of an illustrative process for predicting whether a subject will develop IBD in response to administration of an ICI therapy, according to some embodiments of the technology described herein.

FIG. 4 is a flowchart of an illustrative process for determining cell composition percentages based on cell counts determined for a plurality of cells of a biological sample, according to some embodiments of the technology described herein.

FIG. 5 is a flowchart of an illustrative process for determining a G2 score for a blood sample, according to some embodiments of the technology described herein.

FIG. 6 is a schematic diagram of an illustrative computing device with which aspects described herein may be implemented.

FIG. 7A is a receiver operating characteristic (ROC) curve showing the performance of a machine learning model trained to predict, from clinical data for a subject, the likelihood that a subject will experience an irAE in response to administration of an ICI therapy, according to some embodiments of the technology described herein.

FIG. 7B is a plot showing the importance of clinical input features used by the machine learning model of FIG. 7A in predicting the likelihood that the subject will experience an irAE in response to administration of an ICI therapy, according to some embodiments of the technology described herein.

FIG. 8A is an ROC curve showing the performance of a machine learning model trained to predict, from sequencing data, the likelihood that a subject will experience an irAE in response to administration of an ICI therapy, according to some embodiments of the technology described herein.

FIG. 8B is a plot showing the importance of sequencing data input features used by the machine learning model of FIG. 8A in predicting the likelihood that the subject will experience an irAE in response to administration of an ICI therapy, according to some embodiments of the technology described herein.

FIG. 9A is an ROC curve showing the performance of a machine learning model trained to predict, from immune receptor data, the likelihood that a subject will experience an irAE in response to administration of an ICI therapy, according to some embodiments of the technology described herein.

FIG. 9B is a plot showing the importance of immune receptor input features used by the machine learning model of FIG. 9A in predicting the likelihood that the subject will experience an irAE in response to administration of an ICI therapy, according to some embodiments of the technology described herein.

FIG. 10A is an ROC curve showing the performance of a multi-modal machine learning model trained to predict the likelihood that a subject will experience an irAE in response to administration of an ICI therapy, according to some embodiments of the technology described herein.

FIG. 10B is a plot showing the importance of input features used by the machine learning model of FIG. 10A in predicting the likelihood that the subject will experience an irAE in response to administration of an ICI therapy, according to some embodiments of the technology described herein.

FIG. 10C and FIG. 10D are plots showing the performance of the machine learning model of FIG. 10A in predicting the likelihood that the subject will experience an irAE in response to administration of an ICI therapy, according to some embodiments of the technology described herein.

FIG. 11A and FIG. 11B are plots showing the performance of a machine learning model trained to predict whether subject will develop IBD in response to administration of an ICI therapy, according to some embodiments of the technology described herein.

FIG. 11C is a plot showing the importance of human leukocyte antigen (HLA) input features used by the machine learning model of FIG. 11A and FIG. 11B in predicting whether the subject will develop IBD in response to administration of an ICI therapy, according to some embodiments of the technology described herein.

FIG. 12A, FIG. 12B, FIG. 12C, and FIG. 12D are the plots showing the performance of an HLA model in predicting whether a subject will develop colitis in response to administration of an ICI therapy, according to some embodiments of the technology described herein.

FIG. 13A is a plot showing the distribution of subjects with and without severe irAEs in a space defined by cellular composition signatures determined for the subjects, according to some embodiments of the technology described herein.

FIG. 13B is a plot showing the importance of different cell populations to cellular composition signatures used to differentiate between subjects with and without severe irAEs, according to some embodiments of the technology described herein.

FIG. 13C is a plot showing the performance of cellular composition signatures used to distinguish between subjects with and without severe irAEs, according to some embodiments of the technology described herein.

FIG. 14 is a plot showing an independent differential cell population analysis of cellular clusters that distinguished between patients with and without irAEs, according to some embodiments of the technology described herein.

FIG. 15 and FIG. 16 are plots showing the performance of pathway transcriptomic signatures in distinguishing between patients who experienced severe irAEs and patients who did not, according to some embodiments of the technology described herein.

FIG. 17A shows gene signatures calculated by single sample gene set enrichment analysis (ssGSEA) for patients with severe irAEs, according to some embodiments of the technology described herein.

FIG. 17B shows the performance of gene signatures in distinguishing between patients who experienced severe irAEs and patients who did not, according to some embodiments of the technology described herein.

FIG. 18A and FIG. 18B are boxplots showing examples of immune signatures stratified by adverse event presence, according to some embodiments of the technology described herein.

FIG. 19 are boxplots showing that the G5 immunoprofile type signature can be used to distinguish between patients with and without adverse effects, according to some embodiments of the technology described herein.

FIG. 20 shows the performance of an example autoencoder in separating patients with and without severe irAEs, according to some embodiments of the technology described herein.

FIG. 21A, FIG. 21B, and FIG. 21C are scatterplots showing the correlation between embeddings and biological features, according to some embodiments of the technology described herein.

FIG. 22A is a plot showing a differential analysis of blood cell populations in a cohort of patients, according to some embodiments of the technology described herein.

FIG. 22B are boxplots showing that blood cell populations can be used to distinguish between patients with and without severe irAEs, according to some embodiments of the technology described herein.

### DETAILED DESCRIPTION

An immune-related adverse event is a sign, symptom, or disease associated with the administration of an immunotherapy (e.g., an immune checkpoint inhibitor) to a subject. Immune-related adverse events can arise due to a variety of different causes including, for example, immune system hyperactivation, genetic predisposition, cross-reactivity, and pre-existing autoimmune conditions. Subjects with pre-existing autoimmune conditions may be at higher risk of experiencing an immune-related adverse event due to immune dysregulation, excessive T cell activation, and antibody overproduction by B cells. Additionally, human leukocyte antigen (HLA) genetic variability and baseline expression of immune regulatory molecules (e.g., PD-1, CTLA-4, LAG-3, etc.) can also affect susceptibility to immune-related adverse events, with high expression increasing risk.

Due to the variety of different causal factors, an immune-related adverse event may present itself as one of numerous types of adverse events and may have varying degrees of severity. Examples of types of immune-related adverse events include inflammatory bowel disease (IBD), pneumonitis, hepatitis, myocarditis, cytokine release syndrome, systemic inflammatory response syndrome, diabetes mellitus, arthritis, myositis, myasthenia gravis, Guillain-Barre syndrome, nephritis, and hypothyroidism. The severity of an immune-related adverse event may range from non-severe (e.g., mild and moderate) to severe. The degree (e.g., “grade”) of severity may be determined (e.g., by a healthcare provider) based on the subject's symptoms and/or the intervention required for treating the adverse event. For example, a severe immune-related adverse event may include (i) events requiring hospitalization or prolongation of hospitalization, (ii) events that are disabling or limit self-care, (iii) events with life-threatening consequences, (iv) events requiring urgent intervention, and (v) death. Additional examples of types of immune-related adverse events and criteria for grading the severity of immune-related adverse events are described Brahmer, J. R., et al. (“Management of immune-related adverse events in patients treated with immune checkpoint inhibitor therapy: American Society of Clinical Oncology Clinical Practice Guideline.” Journal of Clinical Oncology 36.17 (2018): 1714-1768), which is incorporated by reference herein in its entirety.

Because immune-related adverse events can lead to severe consequences for a subject, ranging from treatment discontinuation to death, the inventors have recognized the importance of accurately predicting whether a subject will experience an immune-related adverse event (e.g., a severe immune-related event) in response to the administration of an immunotherapy. The ability to accurately predict whether a subject will experience an immune-related adverse event can help to inform treatment decisions and manage the subject's care. For example, if the subject is predicted to experience an immune-related adverse event (e.g., a severe immune-related adverse event) in response to administration of an immunotherapy, then the subject may be treated using an alternative treatment option (e.g., instead of the immunotherapy) and/or excluded from a cohort (e.g., a clinical trial cohort) that will be treated with the immunotherapy. Alternatively, the subject may be treated with the immunotherapy, but the prediction may be used to establish additional interventions, such as additional monitoring and prolonged hospitalization, used to manage the adverse event. By contrast, if the subject is not predicted to experience an immune-related adverse event (e.g., a severe immune-related adverse event), then the therapy may be administered to the subject and/or the subject may be selected as a member of a cohort that will be treated with the immunotherapy.

Conventional techniques for predicting whether a subject will experience an immune-related adverse event are unreliable and inaccurate because they fail to comprehensively account for the variety and complexity of underlying factors that contribute to the development of the many different types of immune-related adverse events. In particular, the conventional techniques use biomarkers that are specific to certain types of immune-related adverse events to predict whether a subject will experience those types of events. These biomarkers also lack the predictive power to differentiate subjects at risk of developing severe (versus non-severe) immune-related adverse events. This poses a number of challenges. First, while a particular biomarker may be used to accurately predict whether a subject will develop one type of immune-related adverse event, it may be irrelevant for predicting whether the subject will develop the many other possible types of immune-related adverse events. Thus, conventional techniques that rely on event-specific biomarkers are unreliable for more generally predicting whether a subject will develop any immune-related adverse event. Second, because the conventional techniques lack the predictive power to differentiate between severe and non-severe immune-related adverse events, they cannot be used to make nuanced treatment decisions for a subject such as, for example, administering an immunotherapy to the subject when the subject is predicted to develop a non-severe immune-related adverse event versus foregoing administration of an immunotherapy to the subject when the subject is predicted to develop a severe immune-related adverse event (e.g., death).

Accordingly, the inventors have developed techniques that address the above-described challenges associated with the conventional techniques for predicting whether a subject will experience an immune-related adverse event. The techniques developed by the inventors include: (a) obtaining healthcare data for the subject, and (b) determining, using at least some of the healthcare data, a likelihood that the subject will experience an immune-related adverse event in response to the administration of an immune checkpoint inhibitor (ICI). The healthcare data may include clinical data, sequencing data, and/or immune receptor data for the subject. The healthcare data may be used to determine the likelihood that the subject will experience the immune-related adverse event by processing the healthcare data using multiple machine learning models. For example, this may include: (a) processing the clinical data using a first machine learning model to output a first likelihood that the subject will experience an immune-related adverse event, (b) processing the sequencing data using a second machine learning model to output a second likelihood that the subject will experience an immune-related adverse event, and/or (c) processing the immune receptor data using a third machine learning model to output a third likelihood that the subject will experience an immune-related adverse event. In some embodiments, the first, second, and/or third likelihoods are processed using a fourth machine learning model trained to predict the likelihood that subject will experience the immune-related adverse event.

The techniques developed by the inventors improve the conventional techniques for predicting whether a subject will experience an immune-related adverse event in a number of ways. The first improvement is that, rather than relying on event-specific biomarkers, the techniques developed by the inventors comprehensively account for the various different subject-specific causal factors that lead to the development of different types of immune-related adverse events. For example, the techniques developed by the inventors integrate data from multiple sources including clinical data, sequencing data, and immune receptor data for the subject. This data accounts for many of the underlying causes of immune-related adverse events including, for example, the subject's genetic profile, immune status, and pre-existing autoimmune conditions. By using several different sources of data that account for the underlying causes of immune-related adverse events and are independent of event type, the techniques developed by the inventors can be used to predict whether the subject will develop any immune-related adverse event, regardless of the type. Moreover, relying on several different sources of data enables flexibility; a prediction can be made even if data from a particular modality is missing (e.g., clinical and sequencing data is available for a subject, but immune receptor data is not).

The second improvement is that the techniques developed by the inventors enable differentiation between severe and non-severe immune-related adverse events, thereby informing nuanced treatment decisions for the subject. In particular, the techniques developed by the inventor increase predictive power for differentiating between severe and non-severe adverse events by (a) using different types of data from multiple different sources, and (b) processing the data using multiple different machine learning models trained to differentiate between severe and non-severe immune-related adverse events. For example, as described herein, the techniques developed by the inventors include processing different types of healthcare data (e.g., clinical data, sequencing data, and immune receptor data) using independently trained machine learning models (e.g., first, second, and third machine learning models) to obtain multiple, independent predictions as to whether the subject will experience an immune-related adverse event (e.g., a severe immune-related adverse event). Not only can the individual predictions be used to inform treatment decisions, but they may also be combined using a fourth machine learning model trained to predict, from the multiple predictions output by the first, second, and third models, a likelihood that the subject will experience an immune-related adverse event. This approach is much more robust, and has greater predictive power, than merely relying on the presence of event-specific biomarkers to predict whether a subject will experience a severe versus non-severe immune-related adverse event.

The inventors have additionally developed techniques for predicting whether a subject will develop a specific type of immune-related adverse event in response to administration of an immunotherapy. For example, the techniques may be used to predict whether a subject will develop inflammatory bowel disease (IBD) in response to administration of an ICI therapy. In some embodiments, the techniques for predicting whether a subject will develop IBD in response to administration of an ICI include: (a) obtaining sequencing data for the subject that indicates whether particular human leukocyte antigen (HLA) alleles are present in the subject's genome, (b) providing, an input to a machine learning model, input features obtained from the sequencing data, and (c) processing the input features using the machine learning model to output a likelihood that the subject will develop IBD in response to the administration of the ICI therapy. In some embodiments, the input features include: (i) a first input feature indicative of a number of HLA alleles that are present in the subject's genome and associated with a risk of IBD, (ii) a second input feature indicative of a number of HLA alleles that are present in the subject's genome and not associated with a risk of IBD, and (iii) third input feature(s) indicative of the particular HLA alleles present in the subject's genome. The ability to predict the specific type of immune-related adverse event that a subject is likely to develop is important because it enables improved care management of that subject. For example, when a subject is predicted to develop IBD in response to administration of an ICI, the healthcare provider may implement a care plan to help manage the IBD such as increased monitoring during treatment and/or prolonged hospitalization. Alternatively, the healthcare provider may decide to adjust the treatment of the patient (e.g., forego administration with the ICI).

Following below are descriptions of various concepts related to, and embodiments of, techniques for predicting whether a subject will experience an immune-related adverse event in response to administration of an immunotherapy. It should be appreciated that various aspects described herein may be implemented in any of numerous ways, as the techniques are not limited in any particular manner of implementation. Example details of implementations are provided herein solely for illustrative purposes. Furthermore, the techniques disclosed herein may be used individually or in any suitable combination, as aspects of the technology described herein are not limited to the use of any particular technique or combination of techniques.

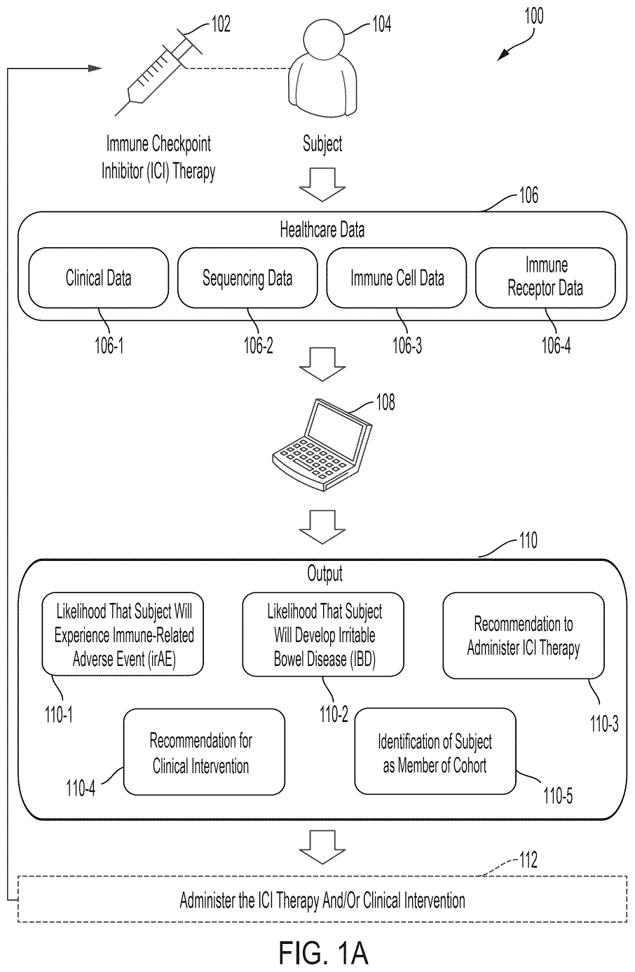

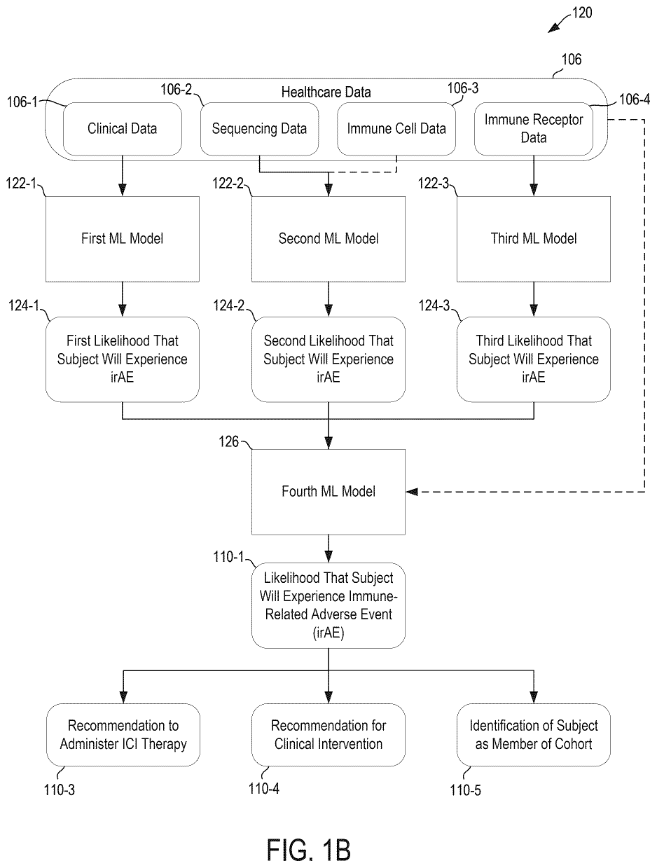

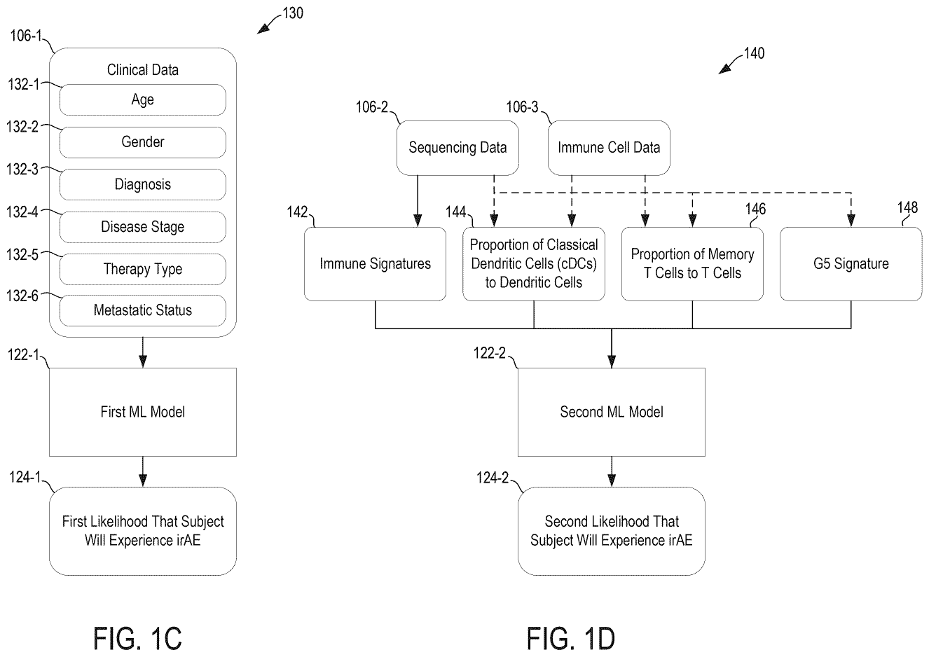

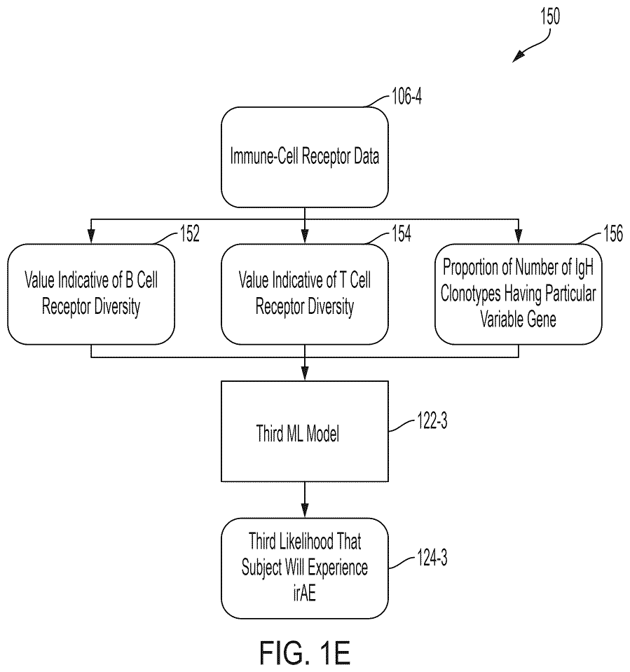

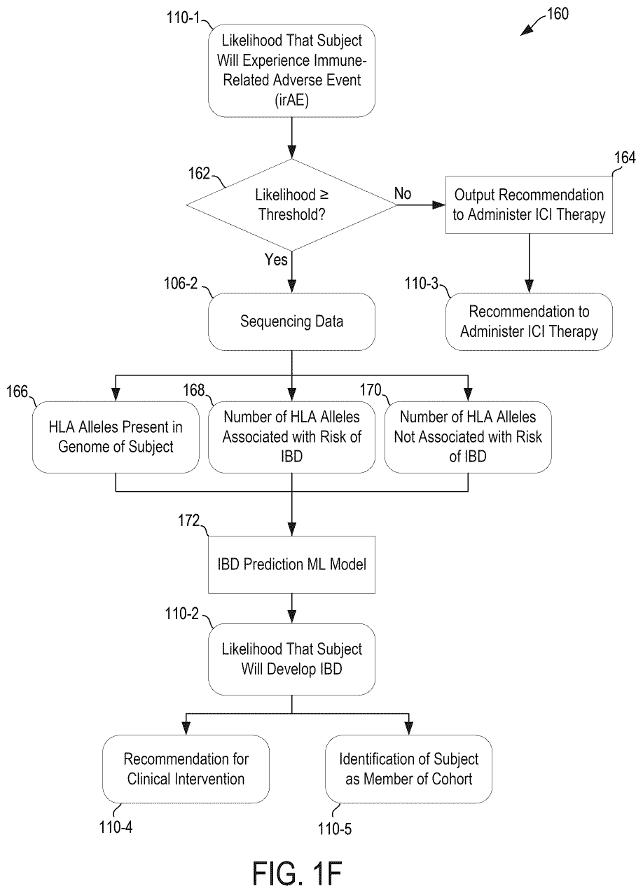

FIG. 1A is a diagram of an illustrative technique 100 for predicting whether a subject 104 will experience an immune-related adverse event (irAE) in response to administration of an immune checkpoint inhibitor (ICI) therapy 102 (e.g., anti-PD-1, anti-PD-L1, and CTLA-4) to the subject 104, according to some embodiments of the technology described herein. Illustrative technique 100 includes obtaining healthcare data 106 for the subject and processing the healthcare data 106 using computing device(s) 108 to obtain output 110. In some embodiments, illustrative technique 100 additionally includes, at act 112, administering the ICI therapy 102 and/or another clinical intervention to the subject 104.

The subject 104 may have, be suspected of having, or be at risk of having cancer. For example, the subject 104 may be diagnosed with cancer. The cancer may be of a particular type. Examples of cancer types include anaplastic astrocytoma, breast neoplasm, colorectal neoplasm, endometrial neoplasm, esophagogastric junction carcinoma, hepatobiliary neoplasm, hepatocellular carcinoma, melanoma, Merkel-cell carcinoma, non-small cell lung carcinoma, renal cell carcinoma, small cell lung carcinoma, squamous cell carcinoma of the head and neck, urinary bladder neoplasm, or any other suitable type of cancer, as aspects of the technology described herein are not limited to a particular cancer type. When the subject has cancer, the cancer may be assigned a stage (e.g., stages I, II, III, or IV) based on characteristics of the cancer. The cancer may be metastatic or not metastatic.

The healthcare data 106 for the subject 104 may include one or more types of healthcare data. For example, as shown in FIG. 1A, the healthcare data may include clinical data 106-1, sequencing data 106-2, immune cell data 106-3, and/or immune receptor data 106-4.

### Clinical Data

The clinical data 106-1 may include health-related information about the subject 104. For example, health-related information may include information about the subject's health status (e.g., diagnoses, conditions, pre-dispositions, etc.), demographics (e.g., age, gender, race, etc.), medical care (e.g., medications, surgeries, treatments, etc.), family history, and/or any other suitable types of health-related information, as aspects of the technology described herein are not limited in this respect. For example, as shown in FIG. 1C, the clinical data 106-1 may include the subject's age 132-1, gender 132-2, diagnosis 132-3, disease stage 132-4, therapy type 132-5, and/or metastatic status 132-6. The diagnosis 132-3 may include one or more of numerous types of diagnoses. For example, the diagnosis 132-3 may include type(s) of cancer with which the subject 104 was diagnosed and examples of which are described herein. The disease stage 132-4 may refer to the stage of cancer (e.g., stages I-IV) with which the subject 104 has been diagnosed. The therapy type(s) 132-5 may include a type of therapy that has already been (or is currently being) administered to the subject 104 and/or a type of therapy (e.g., the ICI 102) to be administered to the subject 104. Examples of therapy types include anti-CTLA-4 with anti-PD-1, anti-PD-1, anti-PD-1 with chemotherapy, anti-PD-1 with other therapy type(s), anti-PD-L1, anti-PD-L1 with chemotherapy, and/or another suitable therapy types, as aspects of the technology described herein are not limited in this respect. The metastatic status 132-6 may indicate whether or not the cancer that the subject 104 has been diagnosed with is metastatic.

The clinical data 106-1 may be obtained, or may have been previously obtained, from the subject's health records (e.g., electronic health records), clinical trial data, insurance claims data, cohort data, billing data, or any other suitable source of clinical data, as aspects of the technology described herein are not limited in this respect.

The sequencing data 106-2, immune cell data 106-3, and immune receptor data 106-4 may be obtained, or may have been previously obtained, from one or more biological samples from the subject 104. The biological sample(s) may be obtained, or may have been previously obtained, by performing a biopsy or by obtaining a blood sample, salivary sample, or any other suitable type of biological sample from the subject. The biological sample(s) may include diseased tissue (e.g., cancerous) and/or healthy tissue. When the biological sample includes a blood sample, the blood sample can be any sample from which blood cell counts (e.g., immune cell counts, peripheral blood mononuclear cell (PBMC) counts, etc.) can be obtained. The origin or preparation methods of the biological sample(s) may include any of the embodiments described herein including with respect to the section entitled “Biological Samples.”

### Sequencing Data

The sequencing data 106-2 may be obtained, or may have been previously obtained, by sequencing a biological sample from the subject. For example, the sequencing data may be obtained using a sequencing platform such as a next generation sequencing platform (e.g., Illumina®, Roche®, Ion Torrent®, etc.), or any high-throughput or massively parallel sequencing platform. In alternative embodiments, the sequencing data may be the result of non-next generation sequencing (e.g., Sanger sequencing). Example techniques for obtaining sequencing data are described herein including at least in the section entitled “Sequencing Data.”

The sequencing data 106-2 may include RNA sequencing data and/or DNA sequencing data. RNA sequencing data may include bulk RNA sequencing (RNA-seq) data, single cell RNA sequencing (scRNA-seq) data, or any other suitable type of RNA sequencing data, as aspects of the technology described herein are not limited in this respect. DNA sequencing data may include whole genome sequencing (WGS) data, whole exome sequencing (WES) data, gene sequencing data, bias-corrected gene sequencing data, or any other suitable type of DNA sequencing data, as aspects of the technology described herein are not limited in this respect. The origin, type, or preparation methods of the sequencing data may include any of the embodiments described herein including at least in the section entitled “Sequencing Data.”

In some embodiments, the sequencing data 106-2 includes data derived from RNA sequencing data and/or DNA sequencing data. For example, the sequencing data 106-2 may include (i) RNA expression data and/or (ii) genotype data.

RNA expression data may be obtained from RNA sequencing data and may include RNA expression levels for one or more genes. The RNA expression data may be obtained by processing the RNA sequencing data in any suitable way and may involve expressing bulk sequencing data in TPM units (or other units) and/or log transforming the RNA expression levels in TPM units. In some embodiments, the RNA expression data includes RNA expression levels for at least 15 genes, at least 20 genes, at least 25 genes, at least 50 genes, at least 75 genes, at least 100 genes, at least 150 genes, at least 200 genes, at least 250 genes, at least 500 genes, at least 1,000 genes, at least 1,500 genes, at least 2,000 genes, at least 2,500 genes, at least 3,000 genes, at least 3,500 genes, at least 4,000 genes, at least 4,500 genes, at least 5,000 genes, at least 6000 genes, at least 7,000 genes, at least 8,000 genes, at least 9,000 genes, at least 10,000 genes, at least 15,000 genes, at least 20,000 genes, or at least any other suitable number of genes, as aspects of the technology described herein are not limited in this respect. In some embodiments, the RNA expression data includes RNA expression levels for at most 15 genes, at most 20 genes, at most 25 genes, at most 50 genes, at most 75 genes, at most 100 genes, at most 150 genes, at most 200 genes, at most 250 genes, at most 500 genes, at most 1,000 genes, at most 1,500 genes, at most 2,000 genes, at most 2,500 genes, at most 3,000 genes, at most 3,500 genes, at most 4,000 genes, at most 4,500 genes, at most 5,000 genes, at most 6000 genes, at most 7,000 genes, at most 8,000 genes, at most 9,000 genes, at most 10,000 genes, at most 15,000 genes, at most 20,000 genes, or at most any other suitable number of genes, as aspects of the technology described herein are not limited in this respect. It should be appreciated that any of the above-listed upper bounds may be coupled with any of the above-listed lower bounds. The origin, type, and/or preparation of the RNA expression data may include any of the embodiments described herein including at least in the section entitled “Sequencing Data.”

Genotype data may be obtained from RNA sequencing data and/or DNA sequencing data. The genotype data may include an indication of one or more alleles present in the subject's genome. For example, as described herein including at least with respect to FIG. 1F, the genotype data may include an indication of one or more human leukocyte antigen (HLA) alleles present in the genome of the subject. In some embodiments, the genotype data is obtained from DNA sequencing data. For example, genotypes may be determined by aligning DNA sequence reads to a reference genome, and determining the genotypes based on the alignment. In alternative embodiments, the genotype data may be obtained from RNA sequencing data. For example, HLA typing may be performed using the arcasHLA tool, which is described by Orenbuch, R., et al. (“arcasHLA: high-resolution HLA typing from RNAseq.” Bioinformatics 36.1 (2020): 33-40), which is incorporated by reference herein in its entirety. It should be appreciated, however, that any other suitable genotyping techniques may be used to obtain genotypes (e.g., HLA allele types), as aspects of the technology described herein are not limited in this respect.

### Immune Cell Data

The immune cell data 106-3 may include information relating to cells in a biological sample (e.g., a blood sample) from the subject. For example, the immune cell data 106-3 may include information relating to the presence, absence, and/or relative amounts of cells in a biological sample.

The immune cell data 106-3 may be obtained, or may have been previously obtained, using an immune platform. For example, the immune cell data 106-3 may be obtained, or may have been previously obtained by processing a blood sample using an immune platform. An immune platform can be any assay and/or a system from which cell type counts can be obtained. For example, an immune platform can be any assay and/or system from which cell type counts can be obtained using cell type specific affinity reagents.

In some embodiments, the immune cell data 106-3 includes cytometry data. For example, the cytometry data may include flow cytometry data, cytometry by time-of-flight (CyTOF) data, and/or spectral cytometry data. The cytometry data may be obtained using an immune platform such as a cytometry platform. For example, the cytometry platform may include any suitable flow cytometry platform. Flow cytometry may be performed using any suitable techniques such as, for example, the techniques described herein including in the section entitled “Flow Cytometry.” Additionally or alternatively, the cytometry platform may include any suitable mass cytometry platform. Mass cytometry may be performed using any suitable techniques such as, for example, the techniques described herein including in the section entitled “Mass Cytometry.” Additionally or alternatively, the cytometry platform may include any suitable spectral cytometry platform. Spectral cytometry may be performed using any suitable techniques such as, for example, the techniques described herein including in the section entitled “Spectral Cytometry.”

In some embodiments, the immune cell data 106-3 includes cell counts obtained using an immune platform such as a hematology analyzer. The hematology analyzer may be configured to count and differentiate between different types of cells in a blood sample. For example, the hematology analyzer may be configured to identify and count basophils, eosinophils, lymphocytes, monocytes, and/or neutrophils. The hematology analyzer may include a commercially available hematology analyzer, such as those available from Sysmex.

In some embodiments, immune cell data 106-3 includes multiplexed immunofluorescence (MxIF) data including one or more MxIF images and/or data derived therefrom. For example, information derived from MxIF images may include information that identifies the location of cells in the image(s) and/or the different types of cells in a blood sample. The MxIF data may include data obtained using an immune platform such as an MxIF imaging platform. In some embodiments, a blood sample is stained using one or more fluorescent markers, and the MxIF platform is configured to obtain immunofluorescence images of the blood sample. For example, the MxIF platform may include at least a microscope and a computing device configured to obtain the immunofluorescence images.

### Immune Receptor Data

The immune receptor data 106-4 may include data about receptors of immune cells (e.g., B cells and T cells) in a biological sample from a subject. For example, the immune receptor data 106-4 may include information about B cell receptors and/or T cell receptors. The information about the B cell receptors and/or T cell receptors may include information about the chains of the B cell receptors and/or T cell receptors. For example, a B cell receptor includes immunoglobulin heavy chains and immunoglobulin light chains. The immunoglobulin light chains of a particular B cell receptor include kappa or lambda chains. A T cell receptor includes alpha chains and beta chains. The information about the chains of the B cell receptors and/or T cell receptors may include information about genes (e.g., variable, diversity, and joining (V(D)J) gene segments) that encode the chains of the B cell receptors and/or T cell receptors. Different B cells within the same biological sample may have different V(D)J gene segments encoding the same type of chain (e.g., immunoglobulin heavy chain, kappa chain, and lambda chains). Similarly different T cells within the same biological sample may have different V(D)J gene segments encoding the same type of chain (e.g., alpha and beta chains). Different V(D)J gene segments (e.g., unique nucleotide sequences) encoding the same chain may be referred to as “clonotypes.” In some embodiments, the immune receptor data 106-4 includes an indication of the different clonotypes present in a biological sample from the subject. For example, this may include sequencing data indicating the nucleotide sequence(s) of a particular clonotype of a particular receptor. Examples of sequencing data and techniques for obtaining same are described above. The sequencing data may be processed using one or more clonotype analysis techniques to obtain the indication of the different clonotypes present in the biological sample. For example, the sequencing data (e.g., FASTQ file(s)) may be processed using MiXCR, which is described by Bolotin, D. A., et al. (“MiXCR: software for comprehensive adaptive immunity profiling.” Nature methods 12.5 (2015): 380-381) and is incorporated by reference herein in its entirety. In some embodiments, the resulting data may include B cell receptor sequence data indicating sequences of B cell receptor chain clonotypes and/or T cell receptor sequence data indicating sequences of T cell receptor chain clonotypes.

As shown in FIG. 1B, at least some of the healthcare data 106 is processed using computing device(s) 108. For example, at least some of the healthcare data 106 may be included in one or more files provided as input to the computing device(s) 108. Additionally or alternatively, at least some of the healthcare data 106 may be provided as input by one or more users interacting with the computing device(s) 108. Additionally or alternatively, the computing device(s) 108 may be used to derive at least some of the healthcare data 106 from other healthcare data that is provided as input to computing device(s) 108.

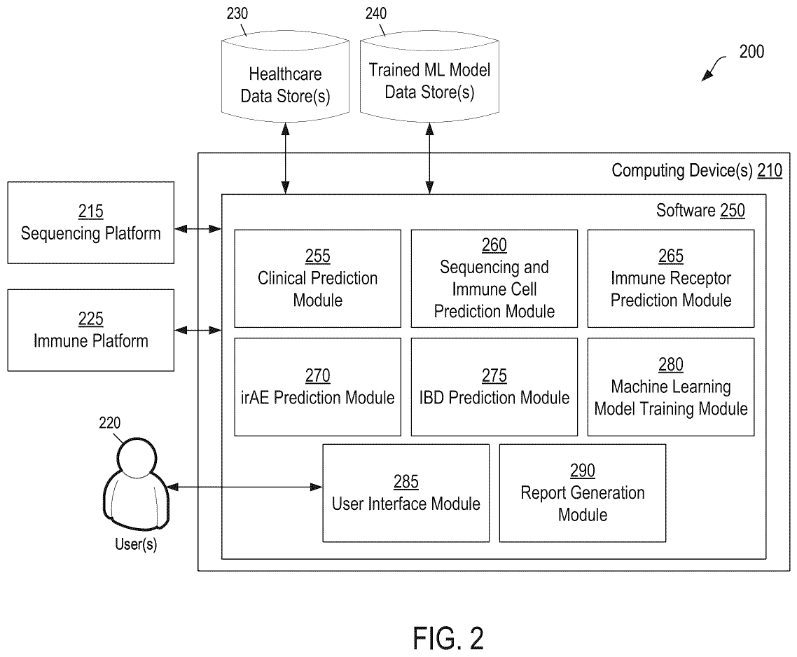

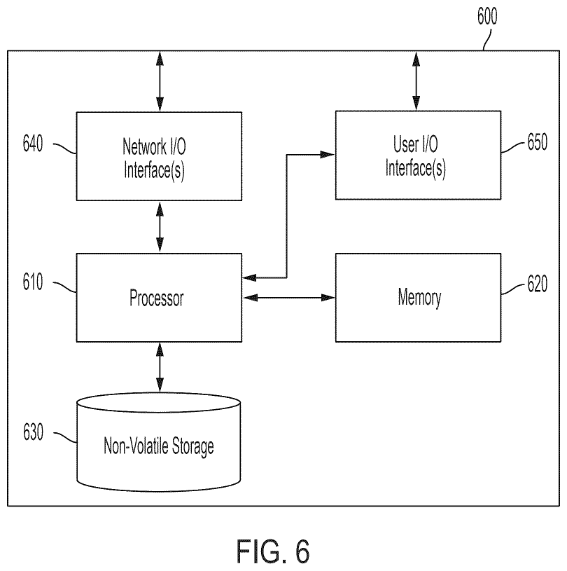

The computing device(s) 108 may include one or more servers, laptops, desktops, smartphones, tablets, cloud instances, virtual machines, computing device(s) 210 described herein with respect to FIG. 2, computing device 600 described herein with respect to FIG. 6, and/or any other suitable type of computing device, as aspects of the technology described herein are not limited in this respect. The computing device(s) 108 may include one or multiple computing devices. When the computing device(s) 108 include multiple computing devices, the multiple computing devices may be configured to communicate via at least one communication network such as the Internet or any other suitable communication network(s), as aspects of the technology described herein are not limited in this respect. For example, the multiple computing devices may be part of a cloud computing environment.

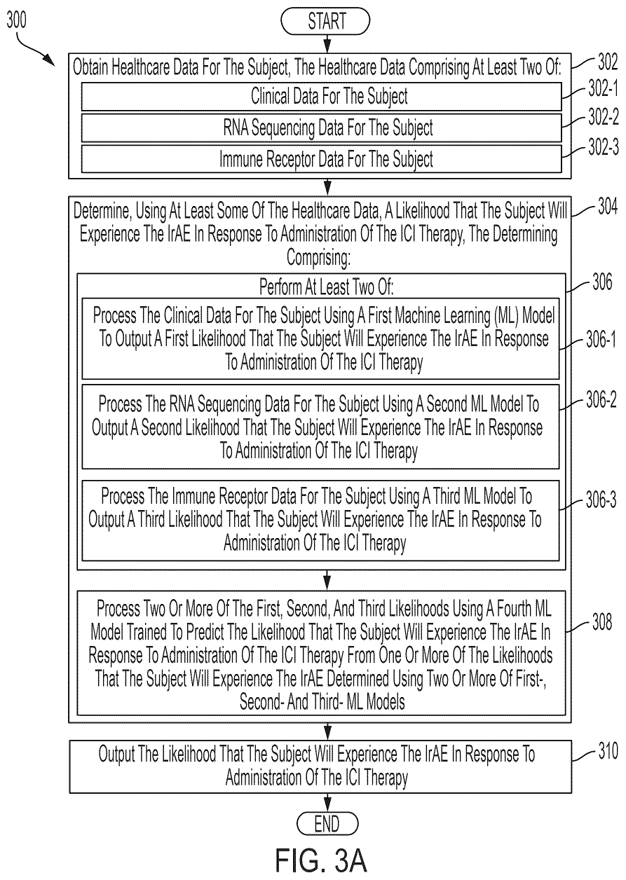

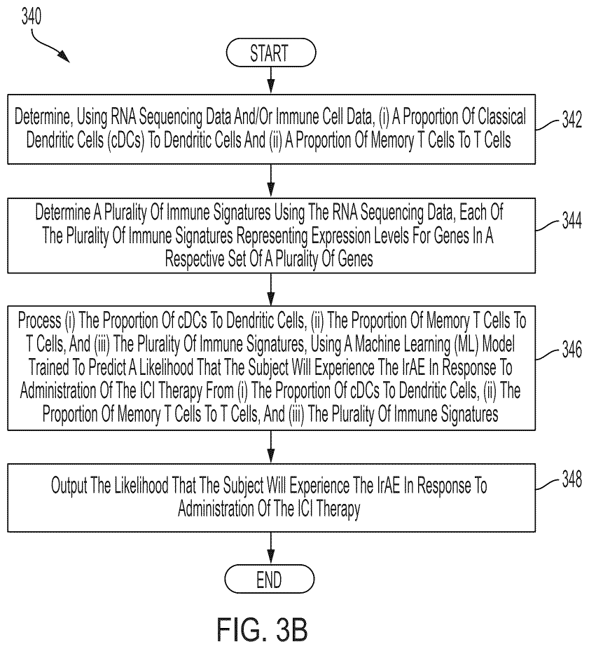

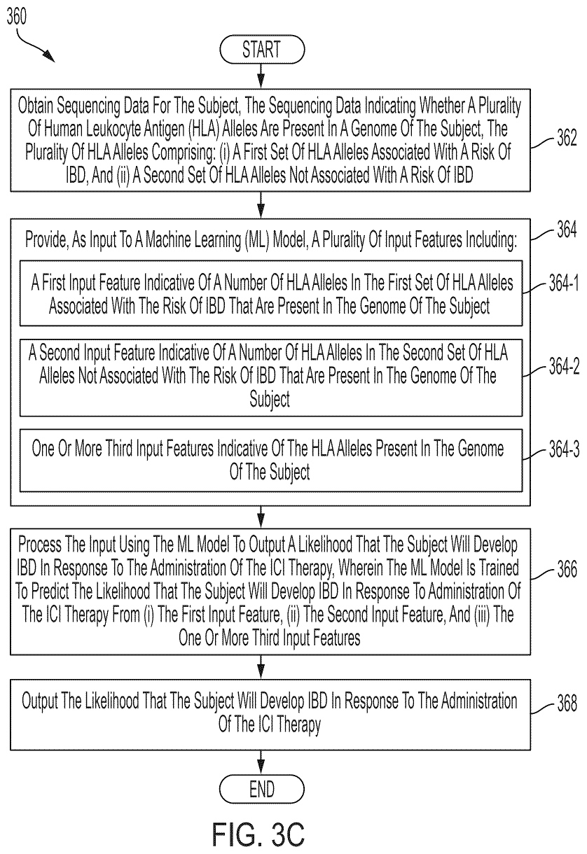

Software (e.g., software 250 shown in FIG. 2) executing on computing device(s) 108 may be configured to process the healthcare data 106. The processing may include: (a) determining, using at least some of the healthcare data 106, a likelihood that the subject will experience an immune-related adverse event in response to administration of an ICI therapy, and (b) outputting the determined likelihood. In some embodiments, determining the likelihood that the subject will experience the immune-related adverse event includes processing at least some of the healthcare data using one or more machine learning models trained to predict respective likelihoods that the subject will experience an immune-related adverse event. Additionally or alternatively, the processing may include (a) determining, using at least some of the healthcare data, a likelihood that the subject will develop IBD in response to administration of an ICI therapy, and (b) outputting the determined likelihood. In some embodiments, determining the likelihood that the subject will develop IBD includes processing at least some of the healthcare data using a machine learning model trained to predict same. Example techniques for processing the healthcare data 106 are described herein including at least with respect to FIGS. 1B-1F and FIGS. 3A-3C.

The computing device(s) 108 may be configured to generate output 110. The output 110 may include results of processing performed by the computing device(s) 108. For example, the output 110 may including: (a) the likelihood (e.g., probability) that the subject will experience an immune-related adverse event (e.g., a severe immune-related adverse event) (output 110-1), and/or (b) the likelihood (e.g., probability) that the subject will develop IBD. In some embodiments, the outputs 110-1 and/or 110-2 may be used by healthcare providers to inform treatment decisions.

Additionally or alternatively, the output 110 may include recommendation(s) for performing one or more follow-up actions. For example, output 110-3 may include a recommendation to administer the ICI therapy 102, (b) output 110-4 may include a recommendation to perform a clinical intervention, and (c) output 110-5 may include a recommendation to identify the subject 104 as a member of a cohort.

In some embodiments, the recommendation to administer the ICI therapy 102, indicated by output 110-3, may be provided when the subject is not predicted to experience an immune-related adverse event or when the subject is not predicted to experience a severe immune-related adverse event. For example, such a recommendation may be provided when the likelihood that the subject will experience an immune-related adverse event or a severe adverse event is less than or equal to a threshold. For example, when using a scale of 0 to 1, with 1 indicating the highest likelihood that the subject will experience an immune-related adverse event, the threshold may be any suitable threshold within the range of 0.3 to 0.9, 0.4 to 0.8, 0.5 to 0.7, or within any other suitable range of likelihoods, as aspects of the technology described herein are not limited in this respect. It should be appreciated, however, that any other suitable scale for measuring likelihoods may be used (e.g., instead of 0 to 1). By contrast, output 110-3 may alternatively include a recommendation to not administer the therapy or to stop administering the therapy. For example, such a recommendation may be provided when the likelihood that the subject will experience an immune-related adverse event or a severe immune-related adverse event is greater than or equal to the threshold.

Output 110-4 may include a recommendation to perform one or more clinical interventions (e.g., other than administering or not administering the ICI therapy). Such a recommendation may be provided when the subject 104 is predicted to experience an immune-related adverse event (e.g., a severe immune-related adverse event). The recommended clinical intervention may be related to monitoring the subject 104 and/or managing the subject's care during administration of the therapy 102 to ensure that healthcare providers address negative healthcare outcomes that may be caused by the immune-related adverse event. For example, output 110-4 may include a recommendation to increase monitoring of the subject 104 during the administration of ICI therapy 102 such as by scheduling more frequent visits with the subject, hospitalizing the subject, prolonging the hospitalization of the subject, and/or checking in with the subject more often. Additionally or alternatively, the output 110-4 may include a recommendation to administer, to the subject 104, one or more treatments (e.g., medications) that will reduce or mitigate symptoms caused by the immune-related adverse event. In some embodiments, the recommendation is specific to the type of immune-related adverse event that the subject is predicted to experience (e.g., IBD).

Output 110-5 may include the identification of the subject 104 (or a recommendation to identify the subject) as a member of a cohort (e.g., a clinical trial cohort). For example, the cohort may include a cohort of subjects that are to be administered the ICI therapy 102 or a cohort of subjects that are not to be administered the ICI therapy 102. In some embodiments, the subject 104 is identified as a member of a cohort that is to be administered the ICI therapy 102 when the subject 104 is not predicted to experience an immune-related adverse event (e.g., a severe immune-related adverse event). For example, the subject 104 may be identified as a member of such a cohort when the likelihood that the subject will experience an immune-related adverse event or a severe adverse event is less than or equal to a threshold, such as the above-described thresholds. By contrast, the subject 104 may be identified as a member of a cohort that is not to be administered the ICI therapy 102 when the subject is predicted to experience an immune-related adverse event (e.g., a severe immune-related adverse event). For example, the subject 104 may be identified as a member of such a cohort when the likelihood that the subject will experience an immune-related adverse event or a severe adverse event is greater than or equal to the threshold.

As shown in FIG. 1A, illustrative technique 100 may additionally include, at optional act 112, administering the ICI therapy 102 and/or another clinical intervention to subject 104. For example, the ICI therapy 102 may be administered to the subject 104 when output 110-3 provides for such a recommendation. Additionally or alternatively, one or more clinical interventions may be performed when output 110-4 provides for such intervention(s).

FIG. 1B is a diagram of an illustrative technique 120 for determining the likelihood 110-1 that the subject 104 will experience an immune-related adverse event in response to administration of the ICI therapy 102, according to some embodiments of the technology described herein. As shown in FIG. 1B, illustrative technique 120 includes processing at least some of the healthcare data 106 using multiple machine learning models (e.g., first machine learning model 122-1, second machine learning model 122-2, third machine learning model 122-3, and fourth machine learning model 126) to obtain the likelihood 110-1 that the subject 104 will experience the immune-related adverse event.

### First Machine Learning Model

The first machine learning model 122-1 is trained to predict, from clinical data 106-1, a first likelihood 124-1 that the subject will experience an immune-related adverse event (e.g., a severe immune-related adverse event). The first machine learning model 122-1 may include any type of machine learning model suitable for predicting a likelihood that a subject will experience an immune-related adverse event. For example, the first machine learning model 122-1 may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. For example, in some embodiments, the first machine learning model 122-1 is a random forest model. Example machine learning models and techniques for training such models are described herein including at least in the sections entitled “Machine Learning” and “Examples.” An example implementation of the first machine learning model 122-1 is described in Section A(2) of the section entitled “Examples.”

FIG. 1C is a diagram of an illustrative technique 130 for determining, from the clinical data 106-1 and using the first machine learning model 122-1, the first likelihood 124-1 that the subject 104 will experience an immune-related adverse event in response to administration of the ICI therapy 102. As shown in FIG. 1C, illustrative technique 130 includes processing, using the first machine learning model 122-1, one or more input features that are included in the clinical data 106-1. For example, the input features include at least some (e.g., all) of the following input features: the subject's age 132-1, gender 132-2, diagnosis 132-3, disease stage 132-4, therapy type 132-5, and metastatic status 132-6. Examples of the clinical data input features are described herein including at least with respect to FIG. 1A.

In some embodiments, illustrative technique 130 includes pre-processing at least some of the clinical data input features before providing them as input to the first machine learning model 122-1. This may include encoding at least some of the input features. The technique for encoding a particular input feature may depend on whether the input feature is categorical or ordinal.

Categorical input features, such as gender 132-2, diagnosis 132-3, therapy type 132-5, and metastatic status 132-6, may be encoded using a first encoding technique. For example, the first encoding technique may include performing one-hot encoding or any other suitable technique for encoding categorical data, as aspects of the technology described herein are not limited in this respect. For example, one-hot encoding may be performed using the get_dummies function in Pandas. In some embodiments, if a particular input feature is missing for the subject 104, the absent input feature is encoded using a placeholder (e.g., −1).

Ordinal input features, such as disease stage 132-4 may be encoded using a second encoding techniques. For example, the second encoding technique may include performing ordinal encoding or any other suitable encoding technique used for preserving ordinality, as aspects of the technology described herein are not limited in this respect. For example, ordinal encoding may be performed using the OrdinalEncoder from Scikit-learn.

One or more (e.g., all) of the input features (e.g., encoded input features) obtained from clinical data 106-1 may be processed using the first machine learning model 122-1 to obtain an output. In some embodiments, the output of the first machine learning model 122-1 includes a likelihood 124-1 (e.g., a probability) that the subject will experience an immune-related adverse event in response to the administration of the ICI therapy 102. Alternatively, the output may include an indication of one of multiple classes for the subject 104. For example, the multiple classes may include at least (i) a first class corresponding to a prediction that the subject 104 will experience an immune-related adverse event in response to administration of the ICI therapy 102, and (ii) a second class corresponding to a prediction that the subject 104 will not experience an immune-related adverse event.

### Second Machine Learning Model

The second machine learning model 122-2 is trained to predict, from sequencing data and (optionally) immune cell data 106-3, a second likelihood 124-2 that the subject will experience an immune-related adverse event (e.g., a severe immune-related adverse event). The second machine learning model 122-2 may include any type of machine learning model suitable for predicting a likelihood that a subject will experience an immune-related adverse event. For example, the second machine learning model 122-2 may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. For example, in some embodiments, the second machine learning model 122-2 is a logistic regression model. Example machine learning models and techniques for training such models are described herein including at least in the sections entitled “Machine Learning” and “Examples.” An example implementation of the second machine learning model 122-2 is described in Section A(3) in the section entitled “Examples.”

FIG. 1D is a diagram of an illustrative technique 140 for determining, from the sequencing data 106-2 and (optionally) immune cell data 106-3 and using the second machine learning model 122-2, the likelihood 124-2 that the subject 104 will experience an immune-related adverse event in response to administration of the ICI therapy 102.

As shown in FIG. 1D, the sequencing data 106-2 and (optionally) immune cell data 106-3 may be used to determine one or more input features to be provided as input to the second machine learning model 122-2. For example, the one or more input features may include (a) immune signatures 142, (b) a proportion 144 of classical dendritic cells (cDCs) to dendritic cells, (c) a proportion 146 of memory T cells to T cells, and/or (d) a G5 signature 148.

The immune signatures 142 may be determined using sequencing data 106-2. For example, the immune signatures 142 may be determined using RNA expression levels for genes in respective gene groups corresponding to the immune signatures 142. Table 1 lists example genes included in gene groups corresponding to the immune signatures 142. In some embodiments, determining a particular immune signature includes using the RNA expression levels to determine an enrichment score for at least some genes in the gene group corresponding to the particular immune signature. For example, with reference to Table 1, the LDHB glycolysis immune signature may be determined by determining an enrichment score for at least some (e.g., all) of the genes included in the LDHB glycolysis signature gene group (row 1 of Table 1). For example, an enrichment score may be determined for at least three, at least four, at least five, or all of the genes listed in a particular gene group. In some embodiments, enrichment scores are determined by performing single sample Gene Score Enrichment Analysis (ssGSEA) using the RNA expression levels for genes in the gene groups. Techniques for performing GSEA are described herein including at least in the section entitled “Expression Data.”

TABLE 1
Example gene groups used for determining immune signatures.

| Gene Group | Genes |
|---|---|
| LDHB glycolysis signature | LDHB, DGKA, GCNT4, TBC1D4, ETS1 |
| Treg and T-cell activation signature | ABCC1, ARID5B, BCL2, BIRC3, CCND2, CCR4, |
| | CD2, CD28, CISH, CTLA4, FAS, FOXP3, GATA3, |
| | ICOS, IL12RB2, IL2RA, IL2RB, LTA, MAF, |
| | MAP3K14, OPTN, P2RY10, PIM2, POU2AF1, |
| | RTKN2, SLAMF1, SOCS1, SOCS2, TIGIT, |
| | TRADD, TRAF1, TRAF2 |
| irAE-associated T-cell signature | TNFRSF4, CD28, KLRB1, TNFRSF18, CD40, |
| | IFNG, TRAT1, EOMES, CD69, CCR8, GZMA, |
| | TIGIT, TNFRSF9, ZAP70, TCF7, KLRK1, ICOS, |
| | CD8B, FASLG, CD27, IKZF2, PRF1, GZMB, |
| | LAIR2, GZMK, CCL5, CD5, GZMH, CD8A, PFKP, |
| | CD40LG, KLRD1, TBX21, NKG7, GNLY, CTLA4, |
| | TRAC |
| Treg signature | FOXP3, CTLA4, IL2RA, CCR8, IKZF4, IKZF2, |
| | RTKN2, CCR4, FAS |
| CD4-related signature | CD28, TCF7, IL2RA, CHMP7, CCR4, CAMK4, |
| | S1PR1, DUSP16, MAL, AQP3, CCR7, RASA3, |
| | CD40LG, GATA3, KCNA3, RCAN3, ZC3H12D, |
| | CD6, LRIG1, TRAF1, TRAT1, CD27, TRABD2A, |
| | TESPA1, ICOS, CACNA1I, ITPKB, PIK3C2B, |
| | TNFRSF10A, CD5 |
| Antigen specific T-cell activation | TESPA1, SIRPG, CD3G, SLAMF6, CD27, LCK, |
| | IKZF3, FCMR, LDLRAP1, LTB, EPB41, LAT, |
| | CD3D, PTPRCAP, ADD3, CD2, MAP4K1, SIT1, |
| | ESYT1, UBASH3A, TRAF3IP3, CD3E, SAMD3, |
| | THEMIS, LIME1, LY9, GRAP, SKAP1, TCF7, |
| | ITM2A, KLRG1 |
| Hypoxia factors signature | FUT11, NDRG1, EPAS1, CA9, LDHA, LOX, |
| | SLC2A1, P4HA1, CA12, HK2, PDK1, PGK1, TPI1, |
| | ALDOA, PFKFB3 |
| LDHA glycolysis signature | HAVCR2, PGK1, LDHA, PSMA6, BPGM, PDIA3, |
| | PDIA6, PLIN2, SPPL2A, LGALS8, YARS, |
| | HSP90B1, MAGT1, SKIL, GSTO1 |
| Platelet signature | ITGA2B, ITGB3, SELP, MPL, GP1BA, GP1BB, |
| | TUBB1 |
| TNF signaling-associated signature | AREG, EREG, LAMB3, PLAU, PTX3 |
| Myeloid suppression signature | TGFB2, IL10, CCL24, CXCL8, S100A12, EBI3, |
| | MSR1, PTGS2, SLC11A1, TREM1, PLAUR |
| M2 polarization signature | TGFB2, TGFB3, IL10, CCL18, IL33, CCL24 |
| Autophagy signature | ATG12, ATG9A, TFEB, RB1CC1, MAP1LC3B, |
| | GABARAPL2, ATG4B, ATG7, GABARAP, VMP1, |
| | ATG14, GABARAPL1, ATG13, NBR1 |

As described herein, the input features for the second machine learning model 122-2 may additionally include the proportion 144 of cDCs to dendritic cells in a biological sample from the subject. The proportion 144 may be determined by: (a) determining the cell composition percentage of cDCs in the biological sample, (b) determining the cell composition percentage of dendritic cells in the biological sample, and (c) determining the proportion of the cell composition percentage of cDCs with respect to the cell composition percentage of dendritic cells. The cell composition percentages may be determined using the sequencing data 106-2 and/or the immune cell data 106-3. Example techniques for determining cell composition percentages for cell types in a biological sample are described herein including at least in the section entitled “Cell Composition Percentages.”

The input features for the second machine learning model 122-2 may additionally include the proportion 146 of memory T cells to T cells. The proportion 146 may be determined by: (a) determining the cell composition percentage of memory T cells in the biological sample, (b) determining the cell composition percentage of T cells in the biological sample, and (c) determining the proportion of the cell composition percentage of memory T cells with respect to the cell composition percentage of T cells. The cell composition percentages may be determined using the sequencing data 106-2 and/or the immune cell data 106-3. Example techniques for determining cell composition percentages for cell types in a biological sample are described herein including at least in the section entitled “Cell Composition Percentages.”

The input features for the second machine learning model 122-2 may additionally include a G5 signature 148. The G5 signature 148 may be indicative of a likelihood that a blood sample obtained from the subject 104 is of a Suppressive (G5) immunoprofile type. An “immunoprofile type” of a blood sample may refer to one of a plurality of immunoprofile types that can be associated with the blood sample, the plurality of immunoprofile types differing by their cell composition percentages for one or more types of immune cells (e.g., one or more types of peripheral blood mononuclear cells (PBMCs)). In some embodiments, a blood sample may be characterized or classified as one of five immunoprofile types. The five immunoprofile types may be described as a Naive type (G1), a Primed type (G2), a Progressive type (G3), a Chronic type (G4), and a Suppressive type (G5). Aspects of immunoprofile types are described herein including at least in the section “Immunoprofile Types.” The G5 signature 148 may be a numerical value that separates samples of the G5 immunoprofile type from samples of non-G5 immunoprofile types (e.g., G1, G2, G3, and G4). For example, the G5 signature 148 may be probability that the blood sample from the subject is of a G5 immunoprofile type. In some embodiments, the G5 signature 148 is a value between 0 and 1.

In some embodiments, the G5 signature 148 is determined using the sequencing data 106-2 and/or immune cell data 106-3. For example, the sequencing data 106-2 and/or immune cell data 106-3 may be used to determine cell composition percentages for a plurality of cell types, and the cell composition percentages may be used to determine the G5 signature. In some embodiments, determining the G5 signature 148 using the cell composition percentages includes (a) normalizing the cell composition percentages relative to a percentage of PBMCs in the blood sample (e.g., the total percentage of PBMCs of all types, or a sum of percentages of PBMCs of a plurality of types), (b) normalizing the cell composition percentages with respect to corresponding cell composition percentages in training data comprising a plurality of training samples, (c) determining an (unnormalized) G5 signature for the blood sample using the normalized cell composition percentages and a G5 statistical model, and (c) (optionally) normalizing the (unnormalized) G5 signature using G5 signatures obtained for the training samples. Aspects of determining a G5 signature for a subject using cell composition percentages are described herein including at least in the section “Immunoprofile Type Signatures.”

One or more (e.g., all) of the input features obtained from sequencing data 106-2 and/or immune cell data 106-3 may be processed using the second machine learning model 122-2 to obtain an output. The input features may be provided, as input to the second machine learning model 122-2, as continuous variables. In some embodiments, the output of the second machine learning model 122-2 includes a likelihood 124-2 (e.g., a probability) that the subject will experience an immune-related adverse event in response to the administration of the ICI therapy 102. Alternatively, the output may include an indication of one of multiple classes for the subject 104. For example, the multiple classes may include at least (i) a first class corresponding to a prediction that the subject 104 will experience an immune-related adverse event in response to administration of the ICI therapy 102, and (ii) a second class corresponding to a prediction that the subject 104 will not experience an immune-related adverse event.

### Third Machine Learning Model

The third machine learning model 122-3 is trained to predict, from the immune receptor data 106-4, a third likelihood that the subject will experience an immune-related adverse event (e.g., a severe immune-related adverse event). The third machine learning model 122-3 may include any type of machine learning model suitable for predicting a likelihood that a subject will experience an immune-related adverse event. For example, the third machine learning model 122-3 may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. For example, in some embodiments, the third machine learning model 122-3 is a logistic regression model. Example machine learning models and techniques for training such models are described herein including at least in the sections entitled “Machine Learning” and “Examples.” An example implementation of the third machine learning model 122-3 is described in Section A(4) in the section entitled “Examples.” FIG. 1E is a diagram of an illustrative technique 150 for determining, from immune receptor data 106-4 and using the fourth machine learning model 122-4, the likelihood 124-4 that the subject 104 will experience an immune-related adverse event in response to administration of the ICI therapy 102.

As shown in FIG. 1E, the immune receptor data 106-4 may be used to determine one or more input features to be provided as input to the third machine learning model 122-3. For example, the one or more input features may include (a) a value 152 indicative of B cell receptor diversity, (b) a value 154 indicative of T cell receptor diversity, and/or (c) a proportion 156 of a number of immunoglobulin heavy chain (IgH) clonotypes associated with a particular variable gene with respect to all heavy chain clonotypes.

In some embodiments, the value 152 indicative of B cell receptor diversity is determined using B cell receptor sequence data included in the immune receptor data 106-4. As described herein, the B cell receptor sequence data may indicate clonotypes (e.g., sequences of V(D)J segments) encoding B cell receptor chains. In some embodiments, the value 152 indicative of B cell receptor diversity may be determined by computing the mean Shannon index across B cell receptor chains (e.g., immunoglobulin heavy, kappa, and lambda chains) using the clonotypes indicated by the B cell receptor sequence data. For example, the mean Shannon index may be computed according to:

$$- \frac{1}{N} {\sum }_{n = 1}^{N} {\sum }_{i = 1}^{{s}_{N}} {p}_{i , N} ln ( {p}_{i , N} )$$

where: N represents a number of receptor chains (e.g., 3 for immunoglobulin heavy, kappa, and lambda chains); s_{N} represents a number of clonotypes for a particular receptor chain (e.g., heavy, kappa, or lambda), and p_{i,N} represents a proportion of a frequency of a particular clonotype with respect to a frequency of all clonotypes for the particular receptor chain.

In some embodiments, the value 154 indicative of T cell receptor diversity is determined using T cell receptor sequence data included in the immune receptor data 106-4. As described herein, the T cell receptor sequence data may indicate clonotypes (e.g., sequences of V(D)J segments) encoding T cell receptor chains. In some embodiments, the value 154 indicative of T cell receptor diversity may be determined by computing the mean Shannon index across T cell receptor chains (e.g., alpha and beta chains) using the clonotypes indicated by the T cell receptor sequence data. For example, the mean Shannon index may be computed according to:

$$- \frac{1}{N} {\sum }_{n = 1}^{N} {\sum }_{i = 1}^{{s}_{N}} {p}_{i , N} ln ( {p}_{i , N} )$$

where: N represents a number of receptor chains (e.g., 2 for alpha and beta chains); s_{N} represents a number of clonotypes for a particular receptor chain (e.g., alpha or beta chain), and p_{i,N} represents a proportion of a frequency of a particular clonotype with respect to a frequency of all clonotypes for the particular receptor chain.

In some embodiments, B cell receptor sequence data is used to determine the proportion 156 of the number of immunoglobulin heavy chain (IgH) clonotypes associated with a particular variable gene with respect to the total number of all heavy chain clonotypes in a biological sample from the subject. For example, the B cell receptor sequence data may indicate the sequences of different V(D)J segments (e.g., different clonotypes) that encode immunoglobulin heavy chains in the biological sample. The sequence of a V(D)J segment includes the sequence of the variable gene included in the V(D)J segment. Thus, the sequences of the V(D)J segments may be used to determine (a) the number of immunoglobulin heavy chain clonotypes that share a particular variable gene, and (b) the total number of immunoglobulin heavy chain clonotypes. For example, this may include determining the number of immunoglobulin heavy chain clonotypes that share the IgHV4-34 gene relative to the total number of immunoglobulin heavy chain clonotypes present in the biological sample from the subject.

One or more (e.g., all) of the input features obtained from immune receptor data 106-4 may be processed using the third machine learning model 122-3 to obtain an output. The input features may be provided, as input to the third machine learning model 122-3, as continuous variables. In some embodiments, the output of the third machine learning model 122-3 includes a likelihood 124-3 (e.g., a probability) that the subject will experience an immune-related adverse event in response to the administration of the ICI therapy 102. Alternatively, the output may include an indication of one of multiple classes for the subject 104. For example, the multiple classes may include at least (i) a first class corresponding to a prediction that the subject 104 will experience an immune-related adverse event in response to administration of the ICI therapy 102, and (ii) a second class corresponding to a prediction that the subject 104 will not experience an immune-related adverse event.

### Fourth Machine Learning Model

The fourth machine learning model 122-4 is trained to predict, from the first, second, and/or third likelihoods 124-1, 124-2, 124-3 and (optionally) healthcare data 106, the likelihood 110-1 that the subject will experience an immune-related adverse event (e.g., a severe immune-related adverse event). The fourth machine learning model 122-4 may include any type of machine learning model suitable for predicting a likelihood that a subject will experience an immune-related adverse event. For example, the fourth machine learning model 122-4 may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. For example, in some embodiments, the fourth machine learning model 122-4 is a logistic regression model. Example machine learning models and techniques for training such models are described herein including at least in the sections entitled “Machine Learning” and “Examples.” An example implementation of the fourth machine learning model 122-4 is described in Section A(5) of the section entitled “Examples.”

Thus, in some embodiments, illustrative technique 120 includes providing, as input to the fourth machine learning model 126, one or more of the first, second, and third likelihoods 124-1, 124-2, and 124-3. For example, all three likelihoods 124-1, 124-2, and 124-3 may be provided as input to the fourth machine learning model 126.

Additionally or alternatively, at least some of the healthcare data 106 may be provided as input to the fourth machine learning model 126. For example, one or more of the input features 132-1-132-6 obtained from clinical data 106-1, shown in FIG. 1C, may be provided as input to the fourth machine learning model 126. Additionally or alternatively, one or more of the input features 142, 144, 146, 148 obtained from sequencing data 106-2 and/or immune cell data 106-3 may be provided as input to the fourth machine learning model 126. Additionally or alternatively, one or more of the input features 152, 154, 156 obtained from immune receptor data 106-4 may be provided as input to the fourth machine learning model 126.

In some embodiments, the output of the fourth machine learning model 126 includes a likelihood 110-1 (e.g., a probability) that the subject will experience an immune-related adverse event in response to the administration of the ICI therapy 102. Alternatively, the output may include an indication of one of multiple classes for the subject 104. For example, the multiple classes may include at least (i) a first class corresponding to a prediction that the subject 104 will experience an immune-related adverse event in response to administration of the ICI therapy 102, and (ii) a second class corresponding to a prediction that the subject 104 will not experience an immune-related adverse event.

### Other Machine Learning Models

While only four machine learning models are shown in FIG. 1B, it should be appreciated that illustrative technique 120 may include processing the healthcare data 106 using one or more other machine learning models. For example, a fifth machine learning model trained to predict, from cytometry data included in the immune cell data 106-3, a likelihood that the subject will experience an immune-related adverse event (e.g., a severe immune-related adverse event) in response to the administration of the ICI therapy 102. The predicted likelihood (e.g., output by the fifth machine learning model) may be provided as an additional or alternative input to the fourth machine learning model 126.

In some embodiments, the cytometry data is used to determine cell composition percentages of one or more immune cell populations in a blood sample obtained from the subject. The cell composition percentages may be processed using the fifth machine learning model to output the likelihood that the subject will experience an immune-related adverse event in response to the administration of the ICI therapy 102. Examples of immune cell populations for which cell composition percentages may be determined include: leukocytes, PBMC, granulocytes, monocytes, dendritic cells, B cells, NK cells, T cells, NKT cells, myeloid-derived suppressor cells (MDSCs), innate lymphoid cells (ILCs), naive B cells, CD20—memory B cells, C27—memory B cells, non-switched memory B cells, class-switched memory B cells, classical monocytes, non-classical monocytes, plasmacytoid dendritic cells (PDCs), classical dendritic cells (cDCs), CDC1, CDC2, invariant natural killer T (iNKT) cells, γδ t cells, mucosal-associated invariant T (MAIT) cells, CD56⁺CD16⁻ NK cells, immature NK cells, mature NK cells, CD4 T cells, CD8 T cells, CD4 regulatory T cells (Tregs), and CD4 T helper cells. The cell composition percentage determined for a particular cell type may be normalized with respect to its nearest parent population. Example techniques for determining cell composition percentages are described herein including at least in the section entitled “Cell Composition Percentages.”

The fifth machine learning model may include any type of machine learning model suitable for predicting a likelihood that a subject will experience an immune-related adverse event. For example, the fifth machine learning model may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. Example machine learning models and techniques for training such models are described herein including at least in the sections entitled “Machine Learning.”

Additionally or alternatively, a sixth machine learning model may be trained to predict, from human leukocyte antigen (HLA) allele features (e.g., obtained from sequencing data 106-2), a likelihood that the subject will experience an immune-related adverse event (e.g., a severe immune-related adverse event) in response to administration of the ICI therapy 102. The predicted likelihood (e.g., output by the sixth machine learning model) may be provided as an additional or alternative input to the fourth machine learning model 126. Examples of HLA allele features are described herein including at least with respect to FIG. 1F.

The sixth machine learning model may include any type of machine learning model suitable for predicting a likelihood that a subject will experience an immune-related adverse event. For example, the sixth machine learning model may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. Example machine learning models and techniques for training such models are described herein including at least in the sections entitled “Machine Learning.”

### Inflammatory Bowel Disease Prediction

As described herein, in some embodiments, the techniques developed by the inventors include determining whether a subject will develop a specific type of immune-related adverse event (e.g., severe immune-related adverse event) in response to the administration of an ICI therapy. FIG. 1F is a diagram of an illustrative technique 160 for predicting whether the subject 104 will develop inflammatory bowel disease (IBD) in response to administration of the ICI therapy 102, according to some embodiments of the technology described herein.

In some embodiments, illustrative technique 160 includes determining whether the subject 104 will develop IBD when the subject is predicted to experience an immune-related adverse event in response to the administration of the therapy 102 (e.g., using the techniques described herein including at least with respect to FIGS. 1A-1E). In alternative embodiments, illustrative technique 160 may include determining whether the subject 104 will develop IBD regardless of whether the subject is 104 is predicted to experience an immune-related adverse event in response to the administration of the therapy 102.

In embodiments that include predicting whether the subject 104 will develop IBD when the subject 104 is predicted to experience an immune-related adverse event, illustrative technique 160 may begin at act 162. At act 162, illustrative technique 160 includes determining whether the likelihood 110-1 that the subject 104 will experience an immune-related adverse event is greater than or equal to a threshold. As described herein, including at least with respect to FIG. 1A, the threshold may depend on the scale of the predicted likelihood, but may include any suitable threshold for distinguishing between (a) subjects likely to experience an immune-related adverse event and subjects unlikely to experience an immune-related adverse event, or (b) subjects likely to experience a severe immune-related adverse event and subjects not likely to experience a severe immune-related adverse event. For example, when using a likelihood scale of 0 to 1, with 1 indicating the highest likelihood that the subject will experience an immune-related adverse event, the threshold may be any suitable threshold within the range of 0.3 to 0.9, 0.4 to 0.8, 0.5 to 0.7, or within any other suitable range of likelihoods, as aspects of the technology described herein are not limited in this respect. It should be appreciated, however, that any other suitable scale for measuring likelihoods may be used (e.g., instead of 0 to 1).

If, at act 162, the likelihood 110-1 is determined to be less than the threshold, illustrative technique 160 may include outputting, at act 164, the recommendation 110-3 to administer the ICI therapy 102 to the subject 104.

If, at act 162, the likelihood 110-1 is determined to be greater than or equal to the threshold, illustrative technique 160 may proceed to predicting, from the sequencing data 106-2 and using an IBD prediction machine learning model 172, the likelihood 110-2 that the subject will develop IBD. It should be appreciated that, in embodiments that include predicting whether the subject will develop IBD regardless of whether the subject 104 is predicted to experience an immune-related adverse event, illustrative technique 160 may exclude act(s) 162 and/or 164.

As shown in FIG. 1F, one or more input features may be obtained from the sequencing data 106-2 and provided as input to the IBD prediction machine learning model 172. The one or more input features may include: (i) first input features 166 comprising HLA alleles present in the genome of the subject, (ii) a second input feature 168 comprising a number of HLA alleles present in the genome of the subject that are associated with a risk of IBD, and (iii) a number of HLA alleles that are present in the genome of the subject that are not associated with a risk of IBD.

The first input feature 166 may include indications of whether certain HLA alleles are present in the genome of the subject 104. As described herein, the sequencing data 106-2 may include indications of allele types that are present in the genome of the subject 104 (e.g., as obtained by sequencing and/or genotyping a biological sample from the subject). In some embodiments, the HLA alleles used for the first input feature 166 include (i) HLA alleles associated with a risk of IBD (“risk alleles), and/or (ii) HLA alleles not associated with a risk of IBD (“protective alleles”). For example, the HLA alleles associated with a risk of IBD include HLA alleles enriched in cohort(s) of subjects diagnosed with IBD. The HLA alleles not associated with a risk of IBD include HLA alleles enriched in healthy cohort(s) (e.g., cohort(s) containing subjects not diagnosed with IBD). Examples of HLA alleles are listed in Table 2. The first input feature 166 may include an indication, for each of at least some (e.g., at least 3, at least 5, at least 10, at least 15, at least 20, at least 30, all, etc.) of the HLA alleles listed in Table 2, as to whether the particular HLA allele is present in the subject's genome.

The second input feature 168 may include a number of certain HLA alleles that are present in the subject's genome and associated with a risk of IBD. For example, the second input feature 168 may include the number of HLA alleles listed in Table 2 that are both (i) present in the subject's genome (e.g., as indicated by sequencing data 106-2), and (ii) associated with a risk of IBD (e.g., as indicated in Table 2).

The third input feature 170 may include a number of certain HLA alleles that are present in the subject's genome and not associated with a risk of IBD. For example, the third input feature 170 may include the number of HLA alleles listed in Table 2 that are both (i) present in the subject's genome (e.g., as indicated by sequencing data 106-2), and (ii) not associated with a risk of IBD (e.g., as indicated in Table 2).

In some embodiments, the first, second, and/or third input features 166, 168, 170 are processed using the IBD prediction machine learning model 172, which is trained to predict, from the input feature(s), the likelihood that the subject 104 with develop IBD in response to administration of the ICI therapy 102. The IBD prediction machine learning model 172 may include any type of machine learning model suitable for predicting a likelihood that a subject will develop IBD in response to administration of an ICI therapy. For example, the IBD prediction machine learning model 172 may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. For example, in some embodiments, the IBD prediction machine learning model 172 is a gradient-boosted decision tree classifier. For example, the gradient-boosted decision tree classifier may be implemented using a gradient boosting algorithm such as CatBoost, XGBoost, LightGBM, or any other suitable gradient boosting algorithm, as aspects of the technology described herein are not limited in this respect. CatBoost is described by Prokhorenkova, L., et al. (“CatBoost: unbiased boosting with categorical features.” Advances in neural information processing systems 31 (2018).), which is incorporated by reference herein in its entirety. LightGBM is described by Ke, G., et al. (“Lightgbm: A highly efficient gradient boosting decision tree.” Advances in neural information processing systems 30 (2017).), which is incorporated by reference herein in its entirety. XGBoost is described by Chen, T. and Guestrin, C. (“Xgboost: A scalable tree boosting system.” Proceedings of the 22nd acm sigkdd international conference on knowledge discovery and data mining. 2016.), which is incorporated by reference herein in its entirety. Example machine learning models and techniques for training such models are described herein including at least in the sections entitled “Machine Learning” and “Examples.” An example implementation of the fourth machine learning model 122-4 is described in Section A(7) in the section entitled “Examples.”

In some embodiments, the output of the IBD prediction machine learning model 172 includes a likelihood 110-2 (e.g., a probability) that the subject 104 will develop IBD in response to administration of the therapy 102. Alternatively, the output may include an indication of one of multiple classes for the subject 104. For example, the multiple classes may include at least (i) a first class corresponding to a prediction that the subject 104 will develop IBD, and (ii) a second class corresponding to a prediction that the subject 104 will not develop IBD.

As shown in FIG. 1F and as described herein with respect to FIG. 1A, the output of the IBD prediction machine learning model 172 (e.g., the likelihood 110-2) may be used to generate a recommendation 110-4 for clinical intervention and/or to identify the subject 104 as the member of a cohort (act 110-5).

TABLE 2
HLA Alleles.

| HLA Allele | Group |
|---|---|
| DPA1*01:03 | Protective |
| DRA*01:05 | Risk |
| DMA*01:01 | Protective |
| A*02:01 | Risk |
| DPB1*04:01 | Risk |
| DOB*01:04 | Risk |
| DRA*01:02 | Protective |
| DPB1*04:02 | Protective |
| DRB3*02:25 | Risk |
| B*51:01 | Risk |
| B*07:02 | Protective |
| DRB1*01:01 | Protective |
| C*07:01 | Protective |
| DRB3*02:01 | Risk |
| DMB*01:02 | Risk |
| C*06:201 | Risk |
| B*08:01 | Protective |
| DQB1*05:01 | Risk |
| C*03:04 | Protective |
| DRB1*04:01 | Protective |
| E*01:13 | Risk |
| DRB1*01:03 | Risk |
| DQB1*02:01 | Protective |
| DRB1*15:04 | Risk |
| C*02:205Q | Risk |
| DRB1*03:01 | Protective |
| DRB1*15:02 | Risk |
| DQB1*06:352 | Risk |
| DRA*01:07 | Risk |
| DQB1*06:01 | Risk |
| DRB1*04:334 | Risk |
| DRB1*04:07 | Protective |
| DRB3*01:108 | Risk |
| DRB1*11:321 | Risk |
| DQB1*03:518 | Risk |
| DRB1*01:02 | Risk |
| DMA*01:06 | Risk |
| DRB1*07:34 | Risk |
| DRB3*02:191 | Risk |
| B*52:01 | Risk |
| C*12:02 | Risk |
| DMA*01:05 | Risk |
| DRA*01:08 | Risk |
| DQB1*06:395 | Risk |
| DRB1*13:327 | Risk |
| DRA*01:06 | Risk |

FIG. 2 is a block diagram of an example system 200 for predicting whether a subject will experience an immune-related adverse event in response to administration of an ICI therapy to the subject, according to some embodiments of the technology described herein. System 200 includes computing device(s) 210, sequencing platform 215, and immune platform 225. The computing device(s) 210 may be configured to have software 250 execute thereon to perform various functions in connection with predicting whether subject will experience an immune-related adverse event. Software 250 includes a plurality of modules. A module may include processor-executable instructions that, when executed by at least one computer hardware processor, cause the at least one computer hardware processor to perform function(s) of the module. Such modules are sometimes referred to herein as “software modules,” each of which includes processor-executable instructions configured to perform one or more acts of one or more processes, such as processes 300, 340, and 360 shown in FIGS. 3A, 3B, and 3C, respectfully.

The computing device(s) 210 may be operated by one or more users 220. The user(s) 220 may provide input specifying processor or other methods to be performed by the computing device(s) 210. For example, the user(s) 220 may provide input specifying processing to be performed on healthcare data (e.g., healthcare data 106 shown in FIGS. 1A and 1B) obtained for one or more subjects. User(s) 220 may provide input by uploading one or more files, interacting with a user interface module 285, and/or using any other suitable technique for providing input, as aspects of the technology described herein are not limited in this respect.

Software 250 may include one or more modules configured to perform functions in connection with predicting whether a subject will experience an immune-related adverse event in response to administration of an ICI therapy. As shown in FIG. 2, such modules may include a clinical prediction module 255, a sequencing and immune cell prediction module 260, a immune receptor prediction module 265, an immune-related adverse event (irAE) prediction module 270, and an IBD prediction module 275.

The clinical prediction module 255 may be configured to predict, from clinical data (e.g., clinical data 106-1 shown in FIGS. 1A, 1B, and 1C) obtained for a subject, a first likelihood (e.g., first likelihood 124-1 shown in FIGS. 1B and 1C) that the subject will experience an immune-related adverse event in response to administration of an ICI therapy. For example, the clinical prediction module 255 may obtain the clinical data from user(s) 220 and/or healthcare data store(s) 230. In some embodiments, the clinical prediction module 255 is configured to process the obtained clinical data using a machine learning model (e.g., first machine learning model 122-1 shown in FIGS. 1B and 1C) trained to predict the likelihood that the subject will experience the immune-related adverse event from the clinical data. For example, the clinical prediction module 255 may be configured to (a) determine input features (e.g., features 132-1-132-6 shown in FIG. 1C) from the clinical data, and (b) process the input features using the trained machine learning model. The clinical prediction module 255 may obtain the trained machine learning model (e.g., parameters of the trained machine learning model) from the trained ML model data store 240 and/or the machine learning model training module 280. Example techniques for predicting, from clinical data, a likelihood that a subject will experience an immune-related adverse event are described herein including at least with respect to FIGS. 1B, 1C, and 3A.

The sequencing and immune cell prediction module 260 may be configured to predict, from sequencing data (e.g., sequencing data 106-2 shown in FIGS. 1A, 1B, and 1D) and (optionally) immune cell data (e.g., immune cell data 106-3 shown in FIGS. 1A, 1B, and 1D) obtained for a subject, a second likelihood (e.g., second likelihood 124-2 shown in FIGS. 1B and 1D) that the subject will experience an immune-related adverse event in response to administration of an ICI therapy. For example, module 260 may obtain the sequencing data from sequencing platform 215, healthcare data store(s) 230, and/or user(s) 220. Module 260 may obtain immune cell data from immune platform 225, healthcare data store(s) 230, and/or user(s) 220. In some embodiments, the sequencing and immune cell prediction module 260 is configured to process the obtained sequencing data and (optionally) immune cell data using a machine learning model (e.g., second machine learning model 122-2 shown in FIGS. 1B and 1D) trained to predict the likelihood that the subject will experience the immune-related adverse event from the sequencing data and (optionally) immune cell data. For example, the sequencing and immune cell prediction module 260 may be configured to (a) determine input features (e.g., features 142, 144, 146, and 148) from the sequencing data and (optionally) immune cell data, and (b) process the input features using the trained machine learning model. Module 260 may obtain the trained machine learning model (e.g., parameters of the trained machine learning model) from the trained ML model data store 240 and/or the machine learning model training module 280. Example techniques for predicting, from sequencing data and immune cell data, a likelihood that a subject will experience an immune-related adverse event are described herein including at least with respect to FIGS. 1B, 1D, 3A, and 3B.

The immune receptor prediction module 265 may be configured to predict, from immune receptor data (e.g., immune receptor data 106-4) obtained for a subject, a third likelihood (e.g., third likelihood 124-3 shown in FIGS. 1B and 1E) that the subject will experience an immune-related adverse event in response to administration of an ICI therapy. For example, the immune receptor prediction module 265 may obtain the immune receptor data from user(s) 220, healthcare data store(s) 230, and/or sequencing platform 215. In some embodiments, the immune receptor prediction module 265 is configured to process the obtained immune receptor data using a machine learning model (e.g., third machine learning model 122-3 shown in FIGS. 1B and 1E) trained to predict the likelihood that the subject will experience the immune-related adverse event from the immune receptor data. For example, the immune receptor prediction module 265 may be configured to (a) determine input features (e.g., features 152, 154, and 156 shown in FIG. 1E) from the immune receptor data, and (b) process the input features using the trained machine learning model. The immune receptor prediction module 265 may obtain the trained machine learning model (e.g., parameters of the trained machine learning model) from the trained ML model data store 240 and/or the machine learning model training module 280. Example techniques for predicting, from immune receptor data, a likelihood that a subject will experience an immune-related adverse event are described herein including at least with respect to FIGS. 1B, 1E, and 3A.

The irAE prediction module 270 may be configured to predict, from the outputs of other modules and (optionally) healthcare data for the subject, the likelihood (e.g., likelihood 110-1 shown in FIGS. 1A and 1B) that the subject will experience an immune-related adverse event in response to administration of an ICI therapy. For example, the irAE prediction module 270 may obtain a first likelihood output by the clinical prediction module 255, a second likelihood output by the sequencing an immune cell prediction module 260, and/or a third likelihood output by the immune receptor prediction module 265. Additionally, the irAE prediction module 270 may (optionally) obtain healthcare data (and/or input features derived therefrom) from the sequencing platform 215, immune platform 225, healthcare data store(s) 230, module 255, module 260, module 265, and/or user(s) 220. In some embodiments, the irAE prediction module 270 is configured to process the obtained the likelihoods and (optionally) healthcare data (and/or features derived therefrom) using a machine learning model (e.g., fourth machine learning model 126 shown in FIG. 1B) trained to predict the likelihood that the subject will experience the immune-related adverse event from the likelihoods and (optionally) the healthcare data (and/or features derived therefrom). The irAE prediction module 270 may obtain the trained machine learning model (e.g., parameters of the trained machine learning model) from the trained ML model data store 240 and/or the machine learning model training module 280. Example techniques for predicting, from other likelihoods that a subject will experience an immune-related adverse event, a likelihood that a subject will experience an immune-related adverse event are described herein including at least with respect to FIG. 1B.

The IBD prediction module 275 may be configured to predict, from sequencing data (e.g., sequencing data 106-2 shown in FIGS. 1A, 1B, and IF) obtained for a subject, a likelihood that the subject will develop inflammatory bowel disease in response to administration of an ICI therapy. For example, the IBD prediction module 275 may obtain the sequencing data from user(s) 220, healthcare data store(s) 230, and/or sequencing platform 215. In some embodiments, the IBD prediction module 275 is configured to process the obtained sequencing data using a machine learning model (e.g., IBD prediction machine learning model shown in FIG. 1F) trained to predict the likelihood that the subject will develop IBD. For example, the IBD prediction module 275 may be configured to (a) determine input features (e.g., features 166, 168, and 170 shown in FIG. 1F) from the sequencing data, and (b) process the input features using the trained machine learning model. The IBD prediction module 275 may obtain the trained machine learning model (e.g., parameters of the trained machine learning model) from the trained ML model data store 240 and/or the machine learning model training module 280. Example techniques for predicting, from sequencing data, a likelihood that a subject will develop IBD are described herein including at least with respect to FIG. 1F and FIG. 3C.

As shown in FIG. 2, software 250 may additionally include a report generation module 290, user interface module 285, and machine learning model training module 280.

The report generation module 290 may be configured to generate one or more reports. In some embodiments, the one or more reports include results of processing healthcare data using one or more of modules 255, 260, 265, and 270. For example, the one or more reports may indicate one or more likelihoods that the subject will experience an immune-related adverse event in response to administration of an ICI therapy. In some embodiments, the one or more reports include results of processing healthcare data using module 275. For example, the one or more reports may indicate a likelihood that the subject will develop IBD. Additionally or alternatively, the one or more reports may include healthcare data, such as the healthcare data used to determine the reported likelihoods. In some embodiments, the one or more reports include recommendation(s), such as recommendation(s) for a healthcare provider. For example, the recommendation(s) may include a recommendation to administer therapy, a recommendation to forego or stop administering a therapy, a recommendation to perform another type of clinical intervention, and/or a recommendation to include the subject as a member of a cohort.

The user interface module 285 may be configured to generate a user interface (e.g., a graphical user interface (GUI)) through which user(s) 220 may provide input and view information generated by software 250. For example, the user(s) 220 may view reports generated by report generation module 290. In some embodiments, the user interface module 285 may be a webpage or web application accessible through an Internet browser. In some embodiments, user interface module 285 may generate a GUI of an app executing on a user's mobile device. In some embodiments, the user interface module 285 may generate a number of selectable elements through which a user may interact. For example, the user interface module 285 may generate dropdown lists, checkboxes, text fields, or any other suitable element.

The machine learning model training module 280 may be configured to train one or more machine learning models for use in connection with predicting whether a subject will experience an immune-related adverse event. For example, the machine learning model training module 280 may be configured to train a first machine learning model to predict, from clinical data for a subject, a first likelihood that the subject will experience an immune-related adverse event. Additionally or alternatively, the machine learning model training module 280 may be configured to train a second machine learning model to predict, from sequencing data and (optionally) immune cell data for a subject, a second likelihood that the subject will experience an immune-related adverse event. Additionally or alternatively, the machine learning model training module 280 may be configured to train a third machine learning model to predict, from immune receptor data for a subject, a third likelihood that the subject will experience an immune-related adverse event. Additionally or alternatively, the machine learning model training module 280 may be configured to train a fourth machine learning model to predict, from the first, second, and/or third likelihoods predicted for the subject, a likelihood that the subject will experience an immune-related adverse event. Additionally or alternatively, the machine learning model training module 280 may be configured to train an IBD prediction machine learning model to predict, from sequencing data, a likelihood that the subject will develop IBD. Examples of machine learning models and techniques for training same are described herein including at least in the section entitled “Machine Learning.” The machine learning model training module 280 may provide the trained machine learning model(s) to the trained machine learning model data store(s) 240. For example, the machine learning model training module 280 may provide the values of parameters of the machine learning model(s) to the trained machine learning model data store(s) 240 for storage thereon.

As shown in FIG. 2, example system 200 additionally includes healthcare data store(s) 230 and trained ML model data store(s) 240. Each of the data stores 230, 240 includes any suitable type of data store (e.g., a flat file, a database system, a multi-file, etc.) and may store data in any suitable format, as aspects of the technology described herein are not limited in this respect. The data stores may be part of software 250 (not shown) or excluded from software 250, as shown in FIG. 2A.

The healthcare data store(s) 230 include one or more data stores configured to store healthcare data obtained for a subject. For example, the healthcare data store(s) 230 may be configured to store clinical data, sequencing data, immune cell data, and/or immune receptor data. The healthcare data store(s) 230 may additionally or alternatively be configured to store features derived from the healthcare data, such as features that may be provided as input(s) to the machine learning model(s) described herein. Additionally or alternatively, the healthcare data store(s) 230 may be configured to store results of processing the healthcare data such as, for example, likelihoods predicted using modules 255, 260, 265, 270, and 275.

In some embodiments, the trained machine learning model data store(s) 240 includes one or more data stores configured to store one or more trained machine learning models. For example, the trained machine learning model data store(s) 240 may store the machine learning models trained to predict a likelihood that the subject will experience an immune-related adverse event and/or the machine learning model trained to predict whether the subject will develop IBD. In some embodiments, the trained machine learning model data store(s) 240 store parameter values for trained machine learning model(s). When the stored trained machine learning model(s) are loaded and used, for example by modules 255, 260, 265, 270, and 275, the parameter values of the trained machine learning model are loaded and stored in memory using at least one data structure.

As shown in FIG. 2, the example system 200 may additionally include sequencing platform 215 and/or immune platform 225. As described herein, including at least with respect to FIG. 1A, an immune platform 225 can be any assay and/or a system from which cell type counts can be obtained. For example, an immune platform can be any assay and/or system from which cell type counts can be obtained using cell type specific affinity reagents. Examples of immune platforms include a cytometry platform (e.g., flow cytometry, mass cytometry, spectral cytometry, etc.), a MxIF platform, and/or a hematology analyzer. A sequencing platform 215 can include any platform used for obtaining sequencing data. For example, the sequencing platform may be a next generation sequencing platform (e.g., Illumina®, Roche®, Ion Torrent®, etc.), or any high-throughput or massively parallel sequencing platform, or a non-next generation sequencing (e.g., Sanger sequencing) platform.

FIG. 3A is a flowchart of an illustrative process 300 for predicting, from healthcare data for a subject, whether the subject will experience an immune-related adverse event in response to administration of an ICI therapy, according to some embodiments of the technology described herein. One or more (e.g., all) of the acts of process 300 may be performed automatically by any suitable computing device(s). For example, the act(s) may be performed by a laptop computer, a desktop computer, one or more servers, in a cloud computing environment, computing device(s) 108 as described herein including at least with respect to FIG. 1A, computing device(s) 210 as described herein including at least with respect to FIG. 2, computing system 600 as described herein including at least with respect to FIG. 6, and/or in any other suitable way, as aspects of the technology described herein are not limited in this respect.

At act 302, healthcare data is obtained for the subject. In some embodiments, the healthcare data comprises at least two of: (a) clinical data 302-1 for the subject, (b) RNA sequencing data 302-2 for the subject, and (c) immune receptor data 302-3 for the subject. Examples of healthcare data and techniques for obtaining same are described herein including at least with respect to FIG. 1A.

At act 304, the likelihood that the subject will experience the immune-related adverse event in response to the administration of the ICI therapy is determined using at least some of the healthcare data. Example techniques for determining a likelihood that the subject will experience an immune-related adverse event are described herein including at least with respect to FIG. 1B.

Act 304 may include sub-acts 306 and 308. At sub-act 306, illustrative technique includes performing at least two of: (a) processing the clinical data for the subject using a first machine learning model to output a first likelihood that the subject will experience the immune-related adverse event; (b) processing the RNA sequencing data for the subject using a second machine learning model to output a second likelihood that the subject will experience the immune-related adverse event; and (c) processing the immune receptor data for the subject using a third machine learning model to output a third likelihood that the subject will experience the immune-related adverse event. Example techniques for performing sub-act 306 are described herein including at least with respect to FIGS. 1C, 1D, and 1E.

At sub-act 308, two or more of the first, second, and third likelihoods are processed using a fourth machine learning model trained to predict the likelihood that the subject will experience the immune-related adverse event in response to the administration of the ICI therapy from one or more of the likelihoods that the subject will experience the immune-related adverse event determined using two or more of the first-, second-, and third-machine learning models. Examples techniques for performing sub-act 308 are described herein including at least with respect to FIG. 1B.

At act 308, the likelihood that the subject will experience the immune-related adverse event in response to the administration of the ICI therapy is output.

FIG. 3B is a flowchart of an illustrative process 340 for predicting, from sequencing data and/or immune cell data for a subject, whether the subject will experience an irAE in response to administration of an ICI therapy, according to some embodiments of the technology described herein. One or more (e.g., all) of the acts of process 340 may be performed automatically by any suitable computing device(s). For example, the act(s) may be performed by a laptop computer, a desktop computer, one or more servers, in a cloud computing environment, computing device(s) 108 as described herein including at least with respect to FIG. 1A, computing device(s) 210 as described herein including at least with respect to FIG. 2, computing system 600 as described herein including at least with respect to FIG. 6, and/or in any other suitable way, as aspects of the technology described herein are not limited in this respect.

At act 342, RNA sequencing data and/or immune cell data is used to determine: (i) a proportion of classical dendritic cells (cDCs) to dendritic cells and (ii) a proportion of memory T cells to T cells. Example techniques for determining (i) a proportion of cDCs to dendritic cells and (ii) a proportion of memory T cells to T cells are described herein including at least with respect to FIG. 1D.

At act 344, a plurality of immune signatures is determined using the RNA sequencing data. Each of the plurality of immune signatures may represent expression levels for genes in a respective set (e.g., gene group) of a plurality of genes. Example techniques for determining immune signatures are described herein including at least with respect to FIG. 1D.

At act 346, the (i) proportion of cDCs to dendritic cells, (ii) proportion of memory T cells to T cells, and (iii) plurality of immune signatures are processed using a machine learning model trained to predict a likelihood that the subject will experience the irAE in response to administration of the ICI therapy from (i) the proportion of cDCs to dendritic cells, (ii) the proportion of memory T cells to T cells, and (iii) the plurality of immune signatures. Example techniques for processing (i) a proportion of cDCs to dendritic cells, (ii) a proportion of memory T cells to T cells, and (iii) plurality of immune signatures using a trained machine learning model are described herein including at least with respect to FIG. 1D.

At act 348, the likelihood that the subject will experience the immune-related adverse event in response to the administration of the ICI therapy is output.

FIG. 3C is a flowchart of an illustrative process 360 for predicting whether a subject will develop IBD in response to administration of an ICI therapy, according to some embodiments of the technology described herein. One or more (e.g., all) of the acts of process 360 may be performed automatically by any suitable computing device(s). For example, the act(s) may be performed by a laptop computer, a desktop computer, one or more servers, in a cloud computing environment, computing device(s) 108 as described herein including at least with respect to FIG. 1A, computing device(s) 210 as described herein including at least with respect to FIG. 2, computing system 600 as described herein including at least with respect to FIG. 6, and/or in any other suitable way, as aspects of the technology described herein are not limited in this respect.

At act 362, sequencing data is obtained for the subject. The sequence data may indicate whether a plurality of human leukocyte antigen (HLA) alleles are present in a genome of the subject. The plurality of HLA alleles may comprise: (i) a first set of HLA alleles associated with a risk of IBD, and (ii) a second set of HLA alleles not associated with a risk of IBD. Example techniques for obtaining sequencing data for a subject are described herein including at least with respect to FIG. 1A.

At act 364, a plurality of input features are provided as input to a machine learning model. In some embodiments, the plurality of input features include: (a) a first input feature 364-1 indicative of a number of HLA alleles in the first set of HLA alleles associated with the risk of IBD that are present in the genome of the subject, (b) a second input feature 264-2 indicative of a number of HLA alleles in the second set of HLA alleles not associated with the risk of IBD that are present in the genome of the subject, and (c) one or more third input features 264-3 indicative of the HLA alleles present in the genome of the subject. Example techniques determining a plurality of input features and providing the input features as input to the machine learning model are described herein including at least with respect to FIG. 1F.

At act 366, the input is processed using the machine learning model to output a likelihood that the subject will develop IBD in response to the administration of the ICI therapy. In some embodiments, the machine learning model is trained to predict the likelihood that the subject will develop IBD in response to administration of the ICI therapy from (i) the first input feature, (ii) the second input feature, and (iii) the one or more third input features. Example techniques for processing input using the machine learning model to output a likelihood that the subject will develop IBD are described herein including at least with respect to FIG. 1F.

At act 368, the likelihood that the subject will develop IBD in response to the administration of the ICI therapy is output.

### Machine Learning

In some embodiments, the techniques developed by the inventors include using one or more trained machine learning models to predict (i) a likelihood that a subject will experience an immune-related adverse event and (ii) a likelihood that a subject will develop IBD. The machine learning model(s) may include a non-linear regression model (e.g., a logistic regression model), a linear regression model, a support vector machine, a Gaussian mixture model, a random forest model, a decision tree classifier, a gradient boosted decision tree classifier, a neural network model, and/or any other suitable type of machine learning model, as aspects of the technology described herein are not limited in this respect. In some embodiments, the machine learning model(s) may include an ensemble of machine learning models of any suitable type (the machine learning models part of the ensemble may be termed “weak learners”).

As described above, in some embodiments, the machine learning model(s) may be implemented as a decision tree classifier. Any suitable type of decision tree classifier may be used and may be trained using any suitable supervised decision tree learning technique. For example, the decision tree classifier may be trained by the iterative dichotomizer technique (e.g., the ID3 algorithm as described, for example, in Quinlan, J. R. 1986. Induction of Decision Trees. Mach. Learn. 1, 1 (March 1986), 81-106)), the C4.5 technique (e.g., as described, for example, in Quinlan, J. R. C4.5: Programs for Machine Learning. Morgan Kaufmann Publishers, 1993), the classification and regression tree (CART) technique (e.g., as described, for example, in Breiman, Leo; Friedman, J. H.; Olshen, R. A.; Stone, C. J. (1984). Classification and regression trees. Monterey, CA: Wadsworth & Brooks/Cole Advanced Books & Software). It should be appreciated that a decision tree classifier may be trained using any other suitable training method, as aspects of the technology described herein are not limited in this respect.

In some embodiments, a gradient-boosted decision tree classifier may be used. The gradient-boosted decision tree classifier may be an ensemble of multiple decision tree classifiers (sometimes called “weak learners”). The prediction (e.g., classification) generated by the gradient-boosted decision tree classifier is formed based on the predictions generated by the multiple decision trees part of the ensemble. The ensemble may be trained using an iterative optimization technique involving calculation of gradients of a loss function (hence the name “gradient” boosting). Any suitable supervised training algorithm may be applied to training a gradient-boosted decision tree classifier including, for example, any of the algorithms described in Hastie, T.; Tibshirani, R.; Friedman, J. H. (2009). “10. Boosting and Additive Trees”. The Elements of Statistical Learning (2nd ed.). New York: Springer. pp. 337-384. In some embodiments, the gradient-boosted decision tree classifier may be implemented using any suitable publicly available gradient boosting framework such as XGBoost (e.g., as described, for example, in Chen, T., & Guestrin, C. (2016). XGBoost: A Scalable Tree Boosting System. In Proceedings of the 22nd ACM SIGKDD International Conference on Knowledge Discovery and Data Mining (pp. 785-794). New York, NY, USA: ACM). The XGBoost software may be obtained from http://xgboost.ai, for example). Another example framework that may be employed is LightGBM (e.g., as described, for example, in Ke, G., Meng, Q., Finley, T., Wang, T., Chen, W., Ma, W., . . . Liu, T.-Y. (2017). Lightgbm: A highly efficient gradient boosting decision tree. Advances in Neural Information Processing Systems, 30, 3146-3154). The LightGBM software may be obtained from https://lightgbm.readthedocs.io/, for example).

In some embodiments, a neural network classifier may be used. The neural network classifier may be trained using any suitable neural network optimization software. The optimization software may be configured to perform neural network training by gradient descent, stochastic gradient descent, or in any other suitable way. In some embodiments, the Adam optimizer (Kingma, D. and Ba, J. (2015) Adam: A Method for Stochastic Optimization. Proceedings of the 3rd International Conference on Learning Representations (ICLR 2015)) may be used.

In some embodiments, a support vector machine (SVM) may be used. The SVM may be implemented using any suitable techniques such as, for example, any of the techniques described by Cristianini, N., and Shawe-Taylor, J. (“An introduction to support vector machines and other kernel-based learning methods.” Cambridge university press, 2000), which is incorporated by reference herein in its entirety.

In some embodiments, a Gaussian mixture model may be used. The Gaussian mixture model may be implemented using any suitable techniques such as, for example, any of the techniques described by Reynolds, D. (“Gaussian mixture models.” Encyclopedia of biometrics 741.659-663 (2009)), which is incorporated by reference herein in its entirety.

In some embodiments, a random forest model may be used. The random forest model may be implemented using any suitable techniques such as, for example, any of the techniques described by Biau, G. (“Analysis of a random forests model.” The Journal of Machine Learning Research 13.1 (2012): 1063-1095), which is incorporated by reference herein in its entirety.

### Subjects

Aspects of this disclosure relate to biological sample that have been obtained from one or more subjects. In some embodiments, a subject is a mammal (e.g., a human, a mouse, a cat, a dog, a horse, a hamster, a cow, a pig, or other domesticated animal, a farm animal (e.g., livestock), a sport animal, a laboratory animal, a pet, and a primate). In some embodiments, a subject is a human. In some embodiments, a subject is an adult human (e.g., of 18 years of age or older). In some embodiments, a subject is a child (e.g., less than 18 years of age).

### Biological Samples

Aspects of the disclosure relate to techniques for predicting whether a subject will experience an immune-related adverse event. In some embodiments, the prediction is generated based on data obtained from one or more biological samples that have been obtained from a subject.

The biological sample may be from any source in the subject's body including, but not limited to, any fluid such as blood (e.g., whole blood, blood serum, or blood plasma), lymph node, breast, etc. Other source in the subject's body may be from saliva, tears, synovial fluid, cerebrospinal fluid, pleural fluid, pericardial fluid, ascitic fluid, and/or urine, hair, skin (including portions of the epidermis, dermis, and/or hypodermis), oropharynx, laryngopharynx, esophagus, bronchus, salivary gland, tongue, oral cavity, nasal cavity, vaginal cavity, anal cavity, bone, bone marrow, brain, thymus, spleen, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

The biological sample may be any type of sample including, for example, a sample of a bodily fluid, one or more cells, one or more pieces of tissue(s) or organ(s). In some embodiments, the biological sample comprises breast tissue sample of the subject. In some embodiments, a breast tissue sample comprises one or more cell types derived from a breast (e.g., epithelial cells, secretory luminal cells, basal/myoepithelial cells, etc.). In some embodiments, a breast tissue sample comprises tumor cells.

In some embodiments, a tissue sample may be obtained from a subject using a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

A sample of lymph node or blood, in some embodiments, refers to a sample comprising cells, e.g., cells from a blood sample or lymph node sample. In some embodiments, the sample comprises non-cancerous cells. In some embodiments, the sample comprises pre-cancerous cells. In some embodiments, the sample comprises cancerous cells. In some embodiments, the sample comprises blood cells. In some embodiments, the sample comprises lymph node cells. In some embodiments, the sample comprises lymph node cells and blood cells.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

In some embodiments, a sample of blood is collected to obtain the cell-free nucleic acid (e.g., cell-free DNA) in the blood.

In some embodiments, the sample may be from a cancerous tissue or an organ or a tissue or organ suspected of having one or more cancerous cells. In some embodiments, the sample may be from a healthy (e.g., non-cancerous) tissue or organ. In some embodiments, a sample from a subject (e.g., a biopsy from a subject) may include both healthy and cancerous cells and/or tissue. In certain embodiments, one sample will be taken from a subject for analysis. In some embodiments, more than one (e.g., 2, 3, 4, 5, 6, 7, 8, 9, 10, 11, 12, 13, 14, 15, 16, 17, 18, 19, 20, or more) samples may be taken from a subject for analysis. In some embodiments, one sample from a subject will be analyzed. In certain embodiments, more than one (e.g., 2, 3, 4, 5, 6, 7, 8, 9, 10, 11, 12, 13, 14, 15, 16, 17, 18, 19, 20, or more) samples may be analyzed. If more than one sample from a subject is analyzed, the samples may be procured at the same time (e.g., more than one sample may be taken in the same procedure), or the samples may be taken at different times (e.g., during a different procedure including a procedure 1, 2, 3, 4, 5, 6, 7, 8, 9, days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same region (e.g., from the same tumor or area of tissue) or a different region (including, e.g., a different tumor). A second or subsequent sample may be taken or obtained from the subject after one or more treatments, and may be taken from the same region or a different region. As a non-limiting example, the second or subsequent sample may be useful in determining whether the cancer in each sample has different characteristics (e.g., in the case of samples taken from two physically separate tumors in a patient) or whether the cancer has responded to one or more treatments (e.g., in the case of two or more samples from the same tumor prior to and subsequent to a treatment).

Any of the biological samples described herein may be obtained from the subject using any known technique. See, for example, the following publications on collecting, processing, and storing biological samples, each of which is incorporated by reference herein in its entirety: Biospecimens and biorepositories: from afterthought to science by Vaught et al. (Cancer Epidemiol Biomarkers Prev. 2012 February; 21 (2): 253-5), and Biological sample collection, processing, storage and information management by Vaught and Henderson (IARC Sci Publ. 2011; (163): 23-42).

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one form to another form such that the first form is no longer detected at the same level as before degradation.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris·Cl; 0.5 mM EDTA, pH 9.0)) and other coagulants, and Acids Citrate Dextrose (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −8° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

### Methods of Treatment

Aspects of the disclosure relate to techniques for predicting whether a subject will experience an immune-related adverse event in response to administration of a therapeutic agent (e.g., one or more anti-cancer agents, such as one or more immunotherapeutic agents). In some embodiments, the techniques include recommending administration of a therapeutic agent to a subject and/or administering a therapeutic agent to the subject. For example, a therapeutic agent may be recommended and/or administered when the subject is not predicted to experience an immune-related adverse event.

In some embodiments, a therapeutic agent (e.g., an anti-cancer therapeutic agent) is an antibody, an immunotherapy, a radiation therapy, a surgical therapy, and/or a chemotherapy.

Examples of the antibody anti-cancer agents include, but are not limited to, alemtuzumab (Campath), trastuzumab (Herceptin), trastuzumab deruxtecan (Enhertu), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor (e.g., nivolumab (Opdivo), pembrolizumab (Keytruda), atezolizumab (Tecentriq), avelumab (Bavencio), durvalumab (Imfinzi)), a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, Carboplatin or Cisplatin, Docetaxel, Gemcitabine, Nab-Paclitaxel, Paclitaxel, Pemetrexed, and Vinorelbine.

Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

In some embodiments, methods described by the disclosure further comprise a step of administering one or more therapeutic agents to the subject. In some embodiments, a subject is administered one or more (e.g., 1, 2, 3, 4, 5, or more) therapeutic agents.

In some embodiments, a subject is administered an effective amount of a therapeutic agent. “An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., leukocyte immunoprofile type, tumor microenvironment, tumor formation, tumor growth, etc.) may be analyzed.

Generally, for administration of any of the anti-cancer antibodies described herein, an initial candidate dosage may be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner (e.g., a medical doctor) wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays. The dosing regimen (including the therapeutic used) may vary over time.

Dosing of immuno-oncology agents is well-known, for example as described by Louedec et al. Vaccines (Basel). 2020 December; 8 (4): 632. For example, dosages of pembrolizumab, for example, include administration of 200 mg every 3 weeks or 400 mg every 6 weeks, by infusion over 30 minutes.

When the anti-cancer therapeutic agent is not an antibody, it may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, e.g., dose, timing, and/or repetition, will depend on the particular subject and that individual's medical history, as well as the properties of the individual agents (such as the half-life of the agent, and other considerations well known in the art).

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of the cancer, or the predisposition toward cancer.

Alleviating cancer includes delaying the development or progression of the disease, or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. Alternatively, or in addition to the clinical techniques known in the art, development of the disease may be detectable and assessed based on other criteria. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

### Sequencing Data

Aspects of the disclosure relate to techniques for predicting whether a subject will experience an immune-related adverse event from sequencing data and/or RNA expression data obtained for a biological sample from the subject.

The RNA expression data used in methods described herein typically is derived from sequencing data obtained from the biological sample.

The sequencing data may be obtained from the biological sample using any suitable sequencing technique and/or apparatus (e.g., sequencing platform 215 shown in FIG. 2). In some embodiments, the sequencing apparatus used to sequence the biological sample may be selected from any suitable sequencing apparatus known in the art including, but not limited to, Illumina™, SOLid™, Ion Torrent™, PacBio™, a nanopore-based sequencing apparatus, a Sanger sequencing apparatus, or a 454™ sequencing apparatus. In some embodiments, sequencing apparatus used to sequence the biological sample is an Illumina sequencing (e.g., NovaSeq™, NextSeq™, HiSeq™, MiSeq™, or MiniSeq™) apparatus.

In some embodiments, sequencing data and/or expression data comprises more than 5 kilobases (kb). In some embodiments, the size of the obtained sequencing data is at least 10 kb. In some embodiments, the size of the obtained sequencing data is at least 100 kb. In some embodiments, the size of the obtained sequencing data is at least 500 kb. In some embodiments, the size of the obtained sequencing data is at least 1 megabase (Mb). In some embodiments, the size of the obtained sequencing data is at least 10 Mb. In some embodiments, the size of the obtained sequencing data is at least 100 Mb. In some embodiments, the size of the obtained sequencing data is at least 500 Mb. In some embodiments, the size of the obtained sequencing data is at least 1 gigabase (Gb). In some embodiments, the size of the obtained sequencing data is at least 10 Gb. In some embodiments, the size of the obtained sequencing data is at least 100 Gb. In some embodiments, the size of the obtained sequencing data is at least 500 Gb.

In some embodiments, sequencing data and/or RNA expression data is obtained by accessing the sequencing data and/or RNA expression data from at least one computer storage medium on which the sequencing data and/or RNA expression data is stored. Additionally or alternatively, in some embodiments, sequencing data and/or RNA expression data may be received from one or more sources via a communication network of any suitable type. For example, in some embodiments, the sequencing data and/or RNA expression data may be received from a server (e.g., a SFTP server, or Illumina BaseSpace).

The sequencing data and/or RNA expression data obtained may be in any suitable format, as aspects of the technology described herein are not limited in this respect. For example, in some embodiments, the sequencing data and/or RNA expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

In some embodiments, after the sequencing data is obtained, it is processed in order to obtain RNA expression data. RNA expression data may be acquired using any method known in the art including, but not limited to whole transcriptome sequencing, whole exome sequencing, total RNA sequencing, mRNA sequencing, targeted RNA sequencing, RNA exome capture sequencing, next generation sequencing, and/or deep RNA sequencing. In some embodiments, RNA expression data may be obtained using a microarray assay.

In some embodiments, the sequencing data is processed to produce RNA expression data. In some embodiments, RNA sequence data is processed by one or more bioinformatics methods or software tools, for example RNA sequence quantification tools (e.g., Kallisto) and genome annotation tools (e.g., Gencode v23), in order to produce expression data. The Kallisto software is described in Nicolas L Bray, Harold Pimentel, Páll Melsted and Lior Pachter, Near-optimal probabilistic RNA-seq quantification, Nature Biotechnology 34, 525-527 (2016), doi: 10.1038/nbt.3519, which is incorporated by reference in its entirety herein.

In some embodiments, microarray expression data is processed using a bioinformatics R package, such as “affy” or “limma,” in order to produce expression data. The “affy” software is described in Bioinformatics. 2004 Feb. 12; 20 (3): 307-15. doi: 10.1093/bioinformatics/btg405. “affy—analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie M E, Phipson B, Wu D, Hu Y, Law C W, Shi W, Smyth G K “limma powers differential expression analyses for RNA-sequencing and microarray studies.” Nucleic Acids Res. 2015 Apr. 20; 43 (7): e47. 20. doi.org/10.1093/nar/gkv007PMID: 25605792, PMCID: PMC4402510, which is incorporated by reference herein its entirety.

In some embodiments, the expression data is acquired through bulk RNA sequencing. Bulk RNA sequencing may include obtaining expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

Expression data (e.g., indicating expression levels) for a plurality of genes may be used for any of the methods or compositions described herein. Expression data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject. The number of genes which may be examined may be up to and inclusive of all the genes of the subject. In some embodiments, expression levels may be determined for all of the genes of a subject. As a non-limiting example, In some embodiments, expression levels may be obtained for at least 25 genes, at least 50 genes, at least 75 genes, at least 100 genes, at least 150 genes, at least 200 genes, at least 250 genes, at least 500 genes, at least 1,000 genes, at least 1,500 genes, at least 2,000 genes, at least 2,500 genes, at least 3,000 genes, at least 3,500 genes, at least 4,000 genes, at least 4,500 genes, at least 5,000 genes, at least 6000 genes, at least 7,000 genes, at least 8,000 genes, at least 9,000 genes, at least 10,000 genes, at least 15,000 genes, at least 20,000 genes, or at least any other suitable number of genes, as aspects of the technology described herein are not limited in this respect. In some embodiments, expression levels may be obtained for at most 25 genes, at most 50 genes, at most 75 genes, at most 100 genes, at most 150 genes, at most 200 genes, at most 250 genes, at most 500 genes, at most 1,000 genes, at most 1,500 genes, at most 2,000 genes, at most 2,500 genes, at most 3,000 genes, at most 3,500 genes, at most 4,000 genes, at most 4,500 genes, at most 5,000 genes, at most 6000 genes, at most 7,000 genes, at most 8,000 genes, at most 9,000 genes, at most 10,000 genes, at most 15,000 genes, at most 20,000 genes, or at most any other suitable number of genes, as aspects of the technology described herein are not limited in this respect. It should be appreciated that any of the above-listed upper bounds may be coupled with any of the above-listed lower bounds. In some embodiments, As another set of non-limiting examples, the expression data may include, for each set of genes listed in Table 1, expression data for at least some (e.g., all) of the genes included in the particular set of genes.

In some embodiments, processing the sequencing data to obtain RNA expression data from the sequencing data includes normalizing the sequencing data to transcripts per kilobase million (TPM) units. The normalization may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM normalization may be performed using a software package, such as, for example, the germa package. Aspects of the germa package are described in Wu J, Gentry RIwcfJMJ (2021). “germa: Background Adjustment Using Sequence Information. R package version 2.66.0.,” which is incorporated by reference in its entirety herein. In some embodiments, RNA expression level in TPM units for a particular gene may be calculated according to the following formula:

$$A \cdot \frac{1}{Σ ( A )} \cdot {10}^{6} where A = \frac{total reads mapped to gene \cdot {10}^{3}}{gene length in bp}$$

Next, in some embodiments, the RNA expression levels in TPM units may be log transformed.

In some embodiments, the RNA expression levels may not be normalized to transcripts per million units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

In some embodiments, enrichment scores for genes in one or more sets of genes are determined. For example, an enrichment score may be determined for at least some genes in the set of genes listed for the gene groups/immune signatures listed in Table 1. In some embodiments, an enrichment score is generated using a gene set enrichment analysis (GSEA) technique, using RNA expression levels of at least some genes in a set of genes. In some embodiments, using a GSEA technique comprises using single-sample GSEA. Aspects of single sample GSEA (ssGSEA) are described in Barbie et al. Nature. 2009 Nov. 5; 462 (7269): 108-112, the entire contents of which are incorporated by reference herein. In some embodiments, ssGSEA is performed according to the following formula:

$$ssGSEA score = \frac{{\sum }_{i}^{N} {r}_{t}^{1.25}}{{\sum }_{l}^{N} {r}_{t}^{0.25}} - \frac{( M - N + 1 )}{2}$$

where rᵢ represents the rank of the ith gene in expression matrix, where N represents the number of genes in the gene set, and where M represents total number of genes in expression matrix. Additional, suitable techniques of performing GSEA are known in the art and are contemplated for use in the methods described herein without limitation. In some embodiments, an enrichment score is calculated by performing ssGSEA on expression data from a plurality of subjects, for example expression data from one or more cohorts of subjects, such as TCGA, Metabric, FUSCCTNBC, GSE103091, GSE106977, GSE21653, GSE25066, GSE41998, GSE47994, GSE81538, GSE96058, etc., in order to produce a plurality of enrichment scores.

### Flow Cytometry

Aspects of the disclosure relate to predicting whether a subject will experience an immune-related adverse event from immune cell data. In some embodiments, the immune cell data includes cytometry data. In some embodiments, the cytometry data is flow cytometry data.

In some embodiments, a flow cytometry platform may be used to perform flow cytometry investigation of a fluid sample. The fluid sample may include target particles with particular particle attributes. The flow cytometry investigation of the fluid sample may provide a flow cytometry result for the fluid sample.

In some embodiments, the fluid sample may be exposed to a stain or dye that provides response radiation when exposed to investigation excitation radiation that may be measured by the radiation detection system of the flow cytometry platform. In some embodiments, a multiplicity of photodetectors are included in the flow cytometry platform. When a particle passes through the laser beam, time correlated pulses on forward scatter (FSC) and side scatter (SSC) detectors, and possibly also fluorescent emission detectors will occur. This is an “event,” and for each event the magnitude of the detector output for each detector, FSC, SSC and fluorescence detectors is stored. The data obtained comprise the signals measured for each of the light scatter parameters and the fluorescence emissions.

Flow cytometry platforms may further comprise components for storing the detector outputs and analyzing the data. For example, data storage and analysis may be carried out using a computer connected to the detection electronics. For example, the data can be stored logically in tabular form, where each row corresponds to data for one particle (or one event), and the columns correspond to each of the measured parameters. The use of standard file formats, such as an “FCS” file format, for storing data from a flow cytometer facilitates analyzing data using separate programs and/or machines. In some embodiments, the data may be displayed in 2-dimensional (2D) plots for ease of visualization, but other methods may be used to visualize multidimensional data.

In some embodiments, the parameters measured using a flow cytometer may include FSC, which refers to the excitation light that is scattered by the particle along a generally forward direction, SSC, which refers to the excitation light that is scattered by the particle in a generally sideways direction, and the light emitted from fluorescent molecules in one or more channels (frequency bands) of the spectrum, referred to as FL1, FL2, etc., or by the name of the fluorescent dye that emits primarily in that channel.

Both flow and scanning cytometers are commercially available from, for example, BD Biosciences (San Jose, Calif.). Flow cytometry is described in, for example, Landy et al. (eds.), Clinical Flow Cytometry, Annals of the New York Academy of Sciences Volume 677 (1993); Bauer et al. (eds.), Clinical Flow Cytometry: Principles and Applications, Williams & Wilkins (1993); Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); and Practical Shapiro, Flow Cytometry, 4th ed., Wiley-Liss (2003); all incorporated herein by reference. Fluorescence imaging microscopy is described in, for example, Pawley (ed.), Handbook of Biological Confocal Microscopy, 2nd Edition, Plenum Press (1989), incorporated herein by reference.

### Mass Cytometry

Aspects of the disclosure relate to predicting whether a subject will experience an immune-related adverse event from immune cell data. In some embodiments, the immune cell data includes cytometry data. In some embodiments, the cytometry data is mass cytometry data.

In some embodiments, a mass cytometry platform may be used to perform mass cytometry investigation of a fluid sample. The fluid sample may include target particles with particular particle attributes. The mass cytometry investigation of the fluid sample may provide a mass cytometry result for the fluid sample.

In some embodiments, the fluid sample may be exposed to target-specific antibodies labeled with metal isotopes. In some embodiments, elemental mass spectrometry (e.g., inductively coupled plasma mass spectrometry (ICP-MS) and time of flight mass spectrometry (TOF-MS)) is used to detect the conjugated antibodies. For example, elemental mass spectrometry can discriminate isotopes of different atomic weights and measure electrical signals for isotopes associated with each particle or cell. Data obtained for a single cell or particle is considered an “event.”

Mass cytometry platforms may further comprise components for storing the detector outputs and analyzing the data. For example, data storage and analysis may be carried out using a computer connected to the detection elements. The use of standard file formats, such as an “FCS” file format, for storing data from a mass cytometry platform facilitates analyzing data using separate programs and/or machines.

Mass cytometry platforms are commercially available from, for example, Fluidigm (San Francisco, CA). Mass cytometry is described in, for example, Bendall et al., A deep profiler's guide to cytometry, Trends in Immunology, 33 (7), 323-332 (2012) and Spitzer et al., Mass Cytometry: Single Cells, Many Features, Cell, 165 (4), 780-791 (2016), both of which are incorporated by reference herein in their entirety.

### Spectral Cytometry

Aspects of the disclosure relate to predicting whether a subject will experience an immune-related adverse event from immune cell data. In some embodiments, the immune cell data includes cytometry data. In some embodiments, the cytometry data is spectral cytometry data.

In some embodiments, a spectral cytometry platform may be used to perform spectral cytometry investigation of a fluid sample. The fluid sample may include target particles with particular particle attributes. The spectral cytometry investigation of the fluid sample may provide a spectral cytometry result for the fluid sample.

In some embodiments, the fluid sample may be exposed to a stain or dye that provides response radiation when exposed to investigation excitation radiation that may be measured by the radiation detection system of the spectral cytometry platform. In some embodiments, a multiplicity of photodetectors are included in the spectral cytometry platform. When a particle passes through the laser beam, time correlated pulses on forward scatter (FSC) and side scatter (SSC) detectors, and possibly also fluorescent emission detectors will occur. This is an “event,” and for each event the magnitude of the detector output for each detector, FSC, SSC and fluorescence detectors is stored. The data obtained comprise the signals measured for each of the light scatter parameters and the fluorescence emissions.

Compared to conventional spectral cytometry, spectral cytometry may utilize a full spectrum of light to distinguish one fluorophore from another. For example, spectral cytometry may utilize multiple (e.g., all) detectors for all fluorophores.

Spectral cytometry platforms may further comprise components for storing the detector outputs and analyzing the data. For example, data storage and analysis may be carried out using a computer connected to the detection electronics. For example, the data can be stored logically in tabular form, where each row corresponds to data for one particle (or one event), and the columns correspond to each of the measured parameters. The use of standard file formats, such as an “FCS” file format, for storing data from a spectral cytometer facilitates analyzing data using separate programs and/or machines. In some embodiments, the data may be displayed in 2-dimensional (2D) plots for ease of visualization, but other methods may be used to visualize multidimensional data.

### Cell Composition Percentages

Aspects of the disclosure relate to determining cell composition percentages. For example, as described herein including at least with respect to FIG. 1D, cell composition percentages may be used to determine a G5 signature (e.g., G5 signature 148 shown in FIG. 1D). Additionally or alternatively, as described herein including at least with respect to FIG. 1D, cell composition percentages may be used to determine a proportion of cDCs to dendritic cells and/or a proportion of memory T cells to T cells.

As used herein, a “cell composition percentage” refers to the percentage of a particular cell type in a plurality of cells. For example, if 100 cells of a total cell population of 500 cells are identified as being CD4 T cells, the cell composition percentage of CD4 T cells in the population is 20%.

Cell composition percentages can be determined using different techniques. The technique may depend on the type of data obtained for the biological sample. For example, different techniques may be used to obtain cell composition percentages given the following types of data: cytometry data, RNA expression data, hematology data, DNA methylation data, and MxIF image data. Examples of techniques for determining cell composition percentages (“deconvolution”) are described herein. However, it should be appreciated that the techniques developed by the inventors are not limited to any particular deconvolution technique, and any suitable deconvolution technique may be used to determine the cell composition percentages of cell types in the biological sample.

### Cytometry-Based Cell Composition Percentages

In some embodiments, cell composition percentages are determined using cytometry data obtained for a biological sample. For example, this may include applying one or more machine learning models to the cytometry data to obtain cell composition percentages for the cell types. Examples of machine learning models that may be used to process cell population data to obtain cell composition percentages are described, for example in International Application No PCT/US2023/012003, published as WO 2023/147177, filed Jan. 31, 2023, the entire contents of which are incorporated by reference herein. Additionally or alternatively, the cell composition percentages may be determined based on cell counts specified in the cytometry data for different cell types. For example, the cytometry data may processed (e.g., by gating) to determine the cell counts. Determining the cell composition percentage for a particular cell type may include determining a ratio of the number of cells of the particular cell type to a total number of cells specified for the sample.

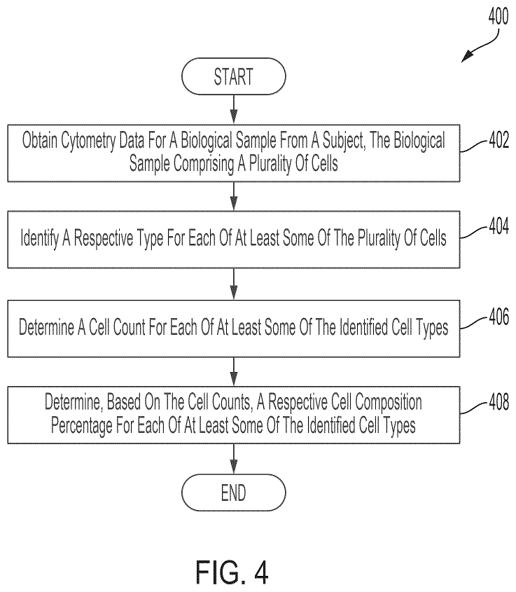

FIG. 4 is a flowchart of process 400 for determining cell composition percentages using cytometry data. Process 400 may be performed in part or in full by a laptop computer, a desktop computer, one or more servers, in a cloud computing environment, computing device as described herein with respect to FIG. 6 or using any other suitable computing device(s), as aspects of the technology described herein are not limited in this respect.

At act 402, cytometry data may be obtained for a biological sample from a subject, the biological sample including a plurality of cells. For example, cytometry (e.g., flow cytometry, mass cytometry, spectral cytometry, etc.) may be performed (or may have previously been performed) on the biological sample (e.g., using any suitable flow cytometry device or platform) to obtain the cytometry data.

Next, at act 404, a respective type is identified for each of at least some of the plurality of cells based on the cytometry data obtained at act 402.

Next, at act 406, a cell count is determined for each of multiple cell types identified at act 404. In some embodiments, this includes determining a number of cells, or cell count, of each type of cell for which cytometry measurements are obtained at act 402. The cell counts, in some embodiments, may be used to determine a number of cells of each type of cell included in at least a hierarchy of cell types. A hierarchy of cell types may indicate relationships between different cell types. For example, the hierarchy of cell types may include parent cell types and cell types that are children, or subtypes, of the parent cell type. In some embodiments, data indicating a hierarchy of cell types is received as input at act 406. Such data may be provided in any suitable format, as aspects of the technology described herein are not limited in this respect.

In some embodiments, data indicating the types identified (at act 404) for each of multiple cells in the biological sample may also be received at act 406. For example, the input may include a tab-separated values file having a number of lines corresponding to the number of objects. Each of at least some of the lines may include an indication of the type determined for the cell. In some embodiments, at least some of the cell types indicated for the cells are included in the hierarchy of cell types. In some embodiments, one or more cell types are not included in the hierarchy of cell types. For example, the identified cell types may include types for “doubles,” which are a combination of two different cell types (e.g., “Monocytes & Neutrophils”). As another example, the identified cell types may include one or more custom cell types which one or more of machine learning models were trained to predict (e.g., “Dead Neutrophils”).

In some embodiments, a “raw” cell count is determined for each unique cell type listed in the data indicating the types identified for the subsample. For example, this includes determining counts for types that are included in the hierarchy of cell types and types that are not included in the hierarchy of cell types.

In some embodiments, the determined cell counts are then updated to conform with cell types included in the hierarchy of cell types. For example, this may include attributing a cell count determined for an identified cell type that is not included in the hierarchy to a cell type that is included in the hierarchy. For example, a cell count determined for the identified cell type of “Dead Neutrophils,” which is not included in the hierarchy, may be attributed to the cell type “Neutrophils,” which is included in the hierarchy. For example, the cell count may be added to the cell count for neutrophils. Accordingly, in some embodiments, since the cell count is accounted for by the “Neutrophil” cell type, the cell count for “Dead Neutrophils” may be discarded. In some embodiments, in updating the determined cell counts to conform with cell types included in the hierarchy of cell types, “doubles” may also be split into two different cell types, and cell counts may be updated for the respective cell types accordingly. For example, a count of “Monocytes & Neutrophils”) may be split into a count of Monocytes and a count of Neutrophils. Accordingly, in some embodiments, any existing cell counts for Monocytes and Neutrophils may be updated to include said counts. Since the cell counts are accounted for by the “Monocyte” and “Neutrophil” cell type, the cell count for “Monocyte & Neutrophil” may be discarded.

In some embodiments, cell counts for parent cell types in the hierarchy of cell types are determined as a sum of the cell counts of their descendants (e.g., subtypes). For example, a cell that is identified to be a “Classical Monocyte” is also a “Monocyte,” since “Classical Monocyte” is a subtype of “Monocyte.” Accordingly, in some embodiments, the cell count of a parent cell type in the hierarchy of cell types may be updated based on the cell counts of its descendants. For example, the cell counts of the descendants may be added to an existing cell count for the parent or added from zero, if there is no existing cell count for the parent cell type. In some embodiments, the techniques for updating cell counts of parent cell types may be carried out sequentially from the bottom of the hierarchy of cell types to the top of the hierarchy of cell types.

Next, at act 408, a cell composition percentage is determined for each of at least some of the identified cell types. In some embodiments, determining a cell composition percentage for a particular cell type includes determining a ratio between the number of cells of a particular type and a total number of cells determined for the biological sample. In some embodiments, determining a cell composition percentage for a particular cell type includes determining a ratio between the number of cells of a particular type and a total number of immune cells determined for the biological sample. In some embodiments, determining a cell composition percentage for a particular cell type includes determining, in the biological sample, a percentage of the particular cell type relative to a cell type class associated with the particular cell type. For example, determining the percentage of naïve T cells relative to the total number of T cells identified in the biological sample. For example, the total number of cells may be determined as the number of leukocytes determined for the biological sample.

In some embodiments, the cell composition percentages determined for particular cell types are used to determine cell concentrations of those cell types in the biological sample. For example, the normalized cell composition percentages may be multiplied by a respective coefficient that converts the cell composition percentage to a cell concentration.

### Expression-Based Cell Composition Percentages

In some embodiments, cell composition percentages are determined using RNA expression data obtained for a biological sample. For example, the cell composition percentages may be determined using one or more cell deconvolution techniques to generate cell composition percentages for one or more cell types. The use of cell deconvolution techniques, for example the BostonGene Kassandra technique, to generate cell composition percentages has been described, for example by International Application No. PCT/US2021/022155, published as International Publication No. WO2021/183917 on Sep. 16, 2021; and International Application No. PCT/US2022/027088, published as International Publication No. WO2022/232615 on Nov. 3, 2022, the entire contents of each of which are incorporated by reference herein. Other cell deconvolution techniques may also be used in methods described by the disclosure, for example Cibersort (e.g., as described by Newman et al. Nature Methods volume 12, pages453-457 (2015)) or CibersortX (e.g., as described by Newman et al. Nature Biotechnology volume 37, pages773-782 (2019)). In some embodiments, more than one cell deconvolution approach is used and then a consensus from the more than one cell devolution approach is used to determine the cell deconvolution.

In some embodiments, the cell composition percentages are adjusted based on a hierarchy of cell types. For example, one or more cell compositions for different cell types may be reconciled with one another.

### DNA Methylation-Based Cell Composition Percentages

In some embodiments, cell composition percentages are determined using DNA methylation data obtained for the biological sample. For example, the cell composition percentages may be determined using a reference-based or a reference-free deconvolution algorithm. An example of a reference-based algorithm is described by Houseman, et al. (Reference-free deconvolution of DNA methylation data and mediation by cell composition effects. BMC Bioinformatics, 17, 259, (2016)), which is incorporated by reference herein in its entirety. Example of reference-free deconvolution algorithms are described by Zou et al. (Epigenome-wide association studies without the need for cell-type composition. Nat. Meth., 11, 309-311, (2014)) and Houseman, et al. (Reference-free cell mixture adjustments in analysis of DNA methylation data. Bioinformatics, 1431-1439, (2014)), each of which is incorporated by reference herein in its entirety.

In some embodiments, the cell composition percentages are adjusted based on a hierarchy of cell types. For example, one or more cell compositions for different cell types may be reconciled with one another.

### Hematology-Based Cell Composition Percentages

In some embodiments, cell composition percentages are determined using hematology data obtained for a biological sample. For example, the cell composition percentages may be determined based on cell counts specified in the hematology data for different cell types. For example, determining a cell composition percentage for a particular cell type may include determining a ratio of the number of cells of the particular cell type to a total number of cells specified for the sample.

In some embodiments, the cell composition percentages are adjusted based on a hierarchy of cell types. For example, one or more cell compositions for different cell types may be reconciled with one another.

### MxIF-Based Cell Composition Percentages

In some embodiments, cell composition percentages are determined using MxIF image data. Example techniques for determining cell composition percentages using MxIF images are described at least by International Application No. PCT/US2021/021265, published as International Publication No. WO2021/178938 on Sep. 10, 2021, and which is incorporated by reference herein in its entirety.

In some embodiments, the cell composition percentages are adjusted based on a hierarchy of cell types. For example, one or more cell compositions for different cell types may be reconciled with one another.

### Immunoprofile Type

In some embodiments, immunoprofile types comprise a Naive type (G1), a Primed type (G2), a Progressive type (G3), a Chronic type (G4), and a Suppressive type (G5). The immunoprofile types (also referred to as PBMC immunoprofile types) described herein may be described by qualitative characteristics, for example by different cell composition percentages for different cell types. In some embodiments, a high cell composition percentage refers to higher cell composition percentage of the same cell type in the subject being analyzed compared to a different subject. In some embodiments, a low cell composition percentage refers to lower cell composition percentage of the same cell type in the subject being analyzed compared to a different subject. In some embodiments, a “high” signal refers to a cell composition percentage that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more increased relative to the cell composition percentage of the same cell type in a different subject. In some embodiments, a “low” signal refers to a cell composition percentage that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more decreased relative to the cell composition percentage of the same cell type in a different subject.

In some embodiments, the Suppressive PMBC immunoprofile type (G5) is characterized by an increased number of myeloid cell populations, including classical monocytes and neutrophils, relative to the other PMBC immunoprofile types.

In some embodiments, the Chronic PMBC immunoprofile type (G4) is characterized by an increased number of CD8 memory and effector cells as well as the NKT cell population, relative to the other PMBC immunoprofile types.

In some embodiments, the Progressive cell memory PMBC immunoprofile type (G3) is characterized by an increased number of CD4 and CD8 memory cells, and high increase in CD8 transitional memory cells, relative to the other PMBC immunoprofile types.

In some embodiments, the Primed PMBC immunoprofile type (G2) is characterized by an increased number of T-helper memory cells, including CD4 central memory, relative to the other PMBC immunoprofile types.

In some embodiments, the Naive PMBC immunoprofile type (G1) is characterized by an increased number of naive CD4, CD8 and B cells, relative to the other PMBC immunoprofile types.

In some embodiments, the immunoprofile types can also be described statistically. For example, each immunoprofile type may correspond to a respective cluster of PBMC signatures obtained for a plurality of training samples, and thus may be described in terms of the PBMC signature clusters. Tables 3-5 describe example PBMC signature clusters. Example aspects of immunoprofile types and selecting an immunoprofile type for a subject are described in International Application No. PCT/US2023/080339, published as International Publication No. WO2024/108156 on May 5, 2023, the entire contents of which are incorporated by reference herein.

TABLE 3

| | G1 | G2 | G3 | G4 | G5 |
|---|---|---|---|---|---|
| | (Naïve) | (Primed) | (Progressive) | (Chronic) | (Suppressive) |
| | 25% | 25% | 25% | 25% | 25% |
| CD4 T cells | 0.516809 | 0.587592 | 0.27119 | 0.261428 | 0.077586 |
| CD4 Naïve T cells | 0.461229 | 0.225648 | 0.122336 | 0.055161 | 0.063215 |
| CD4 Naïve Tregs | 0.315621 | 0.167623 | 0.094701 | 0.052802 | 0.021256 |
| CD4 Memory T | 0.240765 | 0.547057 | 0.262 | 0.330802 | 0.07266 |
| helpers | | | | | |
| CD4 Effector | 0.053214 | 0.131542 | 0.087894 | 0.287602 | 0.049874 |
| Memory | | | | | |
| CD4 Central | 0.246429 | 0.484443 | 0.221505 | 0.188642 | 0.064234 |
| Memory | | | | | |
| CD4 TEMRA | 0.014031 | 0.021267 | 0.010106 | 0.078115 | 0.011735 |
| CD8 T cells | 0.328793 | 0.223175 | 0.182001 | 0.583353 | 0.062078 |
| CD8 Naïve T cells | 0.384364 | 0.086982 | 0.075898 | 0.042453 | 0.044037 |
| CD8 Memory T | 0.13253 | 0.19558 | 0.170497 | 0.630207 | 0.054764 |
| cells | | | | | |
| CD8 Transitional | 0.138353 | 0.205539 | 0.214316 | 0.191516 | 0.051869 |
| Memory | | | | | |
| CD8 Central | 0.107956 | 0.175376 | 0.124022 | 0.122984 | 0.030678 |
| Memory | | | | | |
| CD8 Effector | 0.044591 | 0.064165 | 0.06174 | 0.205876 | 0.02561 |
| Memory | | | | | |
| CD8 TEMRA | 0.030362 | 0.033965 | 0.032092 | 0.45737 | 0.019798 |
| Non-switched | 0.124961 | 0.083798 | 0.040423 | 0.020677 | 0.021477 |
| Memory IgM B | | | | | |
| cells | | | | | |
| Class-switched | 0.145021 | 0.161367 | 0.071409 | 0.054709 | 0.065685 |
| Memory | | | | | |
| Naïve B cells | 0.230684 | 0.187741 | 0.125653 | 0.072578 | 0.103146 |
| Classical | 0.149244 | 0.1827 | 0.320377 | 0.154462 | 0.391395 |
| Monocytes | | | | | |
| Non-classical | 0.093421 | 0.125546 | 0.220434 | 0.132087 | 0.122624 |
| Monocytes | | | | | |
| Mature NK cells | 0.099844 | 0.142419 | 0.222068 | 0.162549 | 0.144145 |
| Immature NK cells | 0.10621 | 0.09467 | 0.1418 | 0.072917 | 0.075758 |
| Dendritic cells | 0.320098 | 0.220471 | 0.32289 | 0.183333 | 0.039343 |
| Plasmacytoid | 0.24047 | 0.157613 | 0.221469 | 0.126741 | 0.033319 |
| Dendritic cells | | | | | |
| NKT cells | 0.083531 | 0.076961 | 0.073639 | 0.387684 | 0.04147 |
| Granulocytes | 0.247181 | 0.303666 | 0.429831 | 0.239702 | 0.789608 |
| Neutrophils | 0.240015 | 0.310561 | 0.398834 | 0.25917 | 0.771303 |
| Basophils | 0.177694 | 0.170987 | 0.214673 | 0.165205 | 0.044676 |
| Eosinophils | 0.106514 | 0.113121 | 0.139433 | 0.085996 | 0.005973 |
| CD4 Tregs | 0.367483 | 0.377588 | 0.244801 | 0.119928 | 0.053491 |
| CD4 Transitional | 0.191683 | 0.352033 | 0.229838 | 0.160369 | 0.051402 |
| Memory | | | | | |
| HLA DR low | 0.02022 | 0.03144 | 0.049407 | 0.023268 | 0.23573 |
| Monocytes | | | | | |
| TIGIT+ PD1+ CD8 | 0.157494 | 0.207882 | 0.207871 | 0.186848 | 0.072178 |
| T cells | | | | | |
| CD39 CD4 Tregs | 0.220702 | 0.315876 | 0.194143 | 0.133377 | 0.124994 |
| gdT Vdelta2+ | 0.064997 | 0.034592 | 0.034595 | 0.022619 | 0.016564 |

TABLE 4

| | G1 | G2 | G3 | G4 | G5 |
|---|---|---|---|---|---|
| | (Naïve) | (Primed) | (Progressive) | (Chronic) | (Suppressive) |
| | Median | Median | Median | Median | Median |
| CD4 T cells | 0.662711 | 0.685517 | 0.366451 | 0.413697 | 0.177509 |
| CD4 Naïve T cells | 0.556319 | 0.35091 | 0.224878 | 0.13328 | 0.130569 |
| CD4 Naïve Tregs | 0.501201 | 0.266506 | 0.190085 | 0.119554 | 0.075814 |
| CD4 Memory T | 0.362402 | 0.648877 | 0.349596 | 0.488784 | 0.184368 |
| helpers | | | | | |
| CD4 Effector | 0.124962 | 0.243893 | 0.161168 | 0.460197 | 0.12102 |
| Memory | | | | | |
| CD4 Central | 0.335085 | 0.603169 | 0.323676 | 0.289721 | 0.147204 |
| Memory | | | | | |
| CD4 TEMRA | 0.040028 | 0.048572 | 0.02456 | 0.208867 | 0.034468 |
| CD8 T cells | 0.467289 | 0.302725 | 0.332053 | 0.696135 | 0.136891 |
| CD8 Naïve T cells | 0.577479 | 0.184101 | 0.182589 | 0.092848 | 0.085994 |
| CD8 Memory T | 0.212876 | 0.288699 | 0.276308 | 0.753472 | 0.147294 |
| cells | | | | | |
| CD8 Transitional | 0.256088 | 0.313295 | 0.340113 | 0.295304 | 0.135786 |
| Memory | | | | | |
| CD8 Central | 0.174808 | 0.296935 | 0.211254 | 0.204562 | 0.083402 |
| Memory | | | | | |
| CD8 Effector | 0.08312 | 0.108541 | 0.126585 | 0.463977 | 0.071121 |
| Memory | | | | | |
| CD8 TEMRA | 0.075175 | 0.09858 | 0.073416 | 0.6324 | 0.079485 |
| Non-switched | 0.195806 | 0.166557 | 0.125041 | 0.070817 | 0.056502 |
| Memory IgM B | | | | | |
| cells | | | | | |
| Class-switched | 0.256662 | 0.269578 | 0.173303 | 0.131577 | 0.135593 |
| Memory | | | | | |
| Naïve B cells | 0.370449 | 0.298478 | 0.245035 | 0.213552 | 0.163953 |
| Classical | 0.225279 | 0.252791 | 0.41498 | 0.269292 | 0.615564 |
| Monocytes | | | | | |
| Non-classical | 0.144156 | 0.204433 | 0.31591 | 0.238835 | 0.279489 |
| Monocytes | | | | | |
| Mature NK cells | 0.176443 | 0.233585 | 0.401355 | 0.254386 | 0.301891 |
| Immature NK cells | 0.17347 | 0.167108 | 0.22399 | 0.157168 | 0.186185 |
| Dendritic cells | 0.437941 | 0.330493 | 0.480443 | 0.316261 | 0.157078 |
| Plasmacytoid | 0.353953 | 0.254119 | 0.378514 | 0.234899 | 0.121252 |
| Dendritic cells | | | | | |
| NKT cells | 0.171432 | 0.175771 | 0.129615 | 0.539552 | 0.129261 |
| Granulocytes | 0.382618 | 0.449489 | 0.561927 | 0.4229 | 0.850685 |
| Neutrophils | 0.387406 | 0.433641 | 0.529458 | 0.40581 | 0.830025 |
| Basophils | 0.262712 | 0.270411 | 0.301113 | 0.248018 | 0.112651 |
| Eosinophils | 0.20403 | 0.215491 | 0.242424 | 0.192835 | 0.066675 |
| CD4 Tregs | 0.492833 | 0.525276 | 0.366742 | 0.218896 | 0.163226 |
| CD4 Transitional | 0.298263 | 0.497258 | 0.321826 | 0.255247 | 0.151531 |
| Memory | | | | | |
| HLA DR low | 0.065281 | 0.07846 | 0.125353 | 0.067239 | 0.477165 |
| Monocytes | | | | | |
| TIGIT+ PD1+ CD8 | 0.240903 | 0.306068 | 0.333351 | 0.342234 | 0.148581 |
| T cells | | | | | |
| CD39 CD4 Tregs | 0.371016 | 0.520242 | 0.372921 | 0.296799 | 0.200762 |
| gdT Vdelta2+ | 0.140606 | 0.083826 | 0.088897 | 0.054894 | 0.050277 |

TABLE 5

| | G1 | G2 | G3 | G4 | G5 |
|---|---|---|---|---|---|
| | (Naïve) | (Primed) | (Progressive) | (Chronic) | (Suppressive) |
| | 75% | 75% | 75% | 75% | 75% |
| CD4 T cells | 0.788032 | 0.786622 | 0.463021 | 0.564684 | 0.366608 |
| CD4 Naïve T cells | 0.741686 | 0.461062 | 0.327475 | 0.260051 | 0.375022 |
| CD4 Naïve Tregs | 0.764426 | 0.408182 | 0.288943 | 0.241674 | 0.185199 |
| CD4 Memory T | 0.465098 | 0.761053 | 0.45632 | 0.655063 | 0.295012 |
| helpers | | | | | |
| CD4 Effector | 0.208899 | 0.378299 | 0.251368 | 0.772331 | 0.27798 |
| Memory | | | | | |
| CD4 Central | 0.466527 | 0.746517 | 0.437682 | 0.411724 | 0.223683 |
| Memory | | | | | |
| CD4 TEMRA | 0.131756 | 0.220494 | 0.058863 | 0.639782 | 0.143112 |
| CD8 T cells | 0.589538 | 0.468197 | 0.48603 | 0.904461 | 0.352648 |
| CD8 Naïve T cells | 0.78544 | 0.323442 | 0.319262 | 0.170799 | 0.192921 |
| CD8 Memory T | 0.320078 | 0.409544 | 0.455537 | 0.915129 | 0.286708 |
| cells | | | | | |
| CD8 Transitional | 0.415027 | 0.441222 | 0.5326 | 0.450074 | 0.244854 |
| Memory | | | | | |
| CD8 Central | 0.263697 | 0.444168 | 0.354911 | 0.280339 | 0.16354 |
| Memory | | | | | |
| CD8 Effector | 0.160068 | 0.198665 | 0.227355 | 0.809943 | 0.127967 |
| Memory | | | | | |
| CD8 TEMRA | 0.176336 | 0.230469 | 0.21788 | 0.907577 | 0.221438 |
| Non-switched | 0.307385 | 0.267483 | 0.227953 | 0.149117 | 0.14205 |
| Memory IgM B | | | | | |
| cells | | | | | |
| Class-switched | 0.42331 | 0.464562 | 0.289808 | 0.246844 | 0.248892 |
| Memory | | | | | |
| Naïve B cells | 0.571189 | 0.43868 | 0.406178 | 0.360336 | 0.335013 |
| Classical | 0.303541 | 0.362069 | 0.559735 | 0.365677 | 0.863046 |
| Monocytes | | | | | |
| Non-classical | 0.252922 | 0.299484 | 0.495174 | 0.390367 | 0.575074 |
| Monocytes | | | | | |
| Mature NK cells | 0.326604 | 0.380774 | 0.617431 | 0.501855 | 0.440678 |
| Immature NK cells | 0.26615 | 0.274601 | 0.364978 | 0.262244 | 0.360084 |
| Dendritic cells | 0.551467 | 0.424051 | 0.646407 | 0.434698 | 0.306728 |
| Plasmacytoid | 0.521473 | 0.349518 | 0.568588 | 0.380915 | 0.2793 |
| Dendritic cells | | | | | |
| NKT cells | 0.264899 | 0.343661 | 0.256366 | 0.866944 | 0.344584 |
| Granulocytes | 0.517676 | 0.595496 | 0.685545 | 0.57367 | 0.991513 |
| Neutrophils | 0.52906 | 0.572942 | 0.656846 | 0.579071 | 0.988562 |
| Basophils | 0.396037 | 0.432851 | 0.431055 | 0.353198 | 0.207963 |
| Eosinophils | 0.333206 | 0.366476 | 0.423774 | 0.333555 | 0.155757 |
| CD4 Tregs | 0.663132 | 0.668141 | 0.478286 | 0.394052 | 0.306773 |
| CD4 Transitional | 0.453336 | 0.648222 | 0.476315 | 0.372905 | 0.282151 |
| Memory | | | | | |
| HLA DR low | 0.140713 | 0.16051 | 0.304512 | 0.217529 | 0.882418 |
| Monocytes | | | | | |
| TIGIT+ PD1+ CD8 | 0.351118 | 0.425046 | 0.545905 | 0.51332 | 0.273978 |
| T cells | | | | | |
| CD39 CD4 Tregs | 0.483579 | 0.682502 | 0.489588 | 0.389691 | 0.367768 |
| gdT Vdelta2+ | 0.28449 | 0.186779 | 0.200473 | 0.103883 | 0.126101 |

### Immunoprofile Type Signatures

Aspects of the disclosure relate to determining a G5 signature for a biological sample by processing immune cell data. For example, the immune cell data may be processed to determine cell composition percentages for at least some cell types in the biological sample, and the cell composition percentages may be used to determine the G5 signature. Example techniques for determining cell composition percentages are described herein including at least in the section “Cell Composition Percentages.” In some embodiments, the G5 signature is a metric that separates samples of the G5 immunoprofile type from samples of non-G5 immunoprofile types (e.g., G1, G2, G3, and G4). Example aspects of immunoprofile types and selecting an immunoprofile type for a subject are described in International Application No. PCT/US2023/080339, published as International Publication No. WO2024/108156 on May 5, 2023, the entire contents of which are incorporated by reference herein.

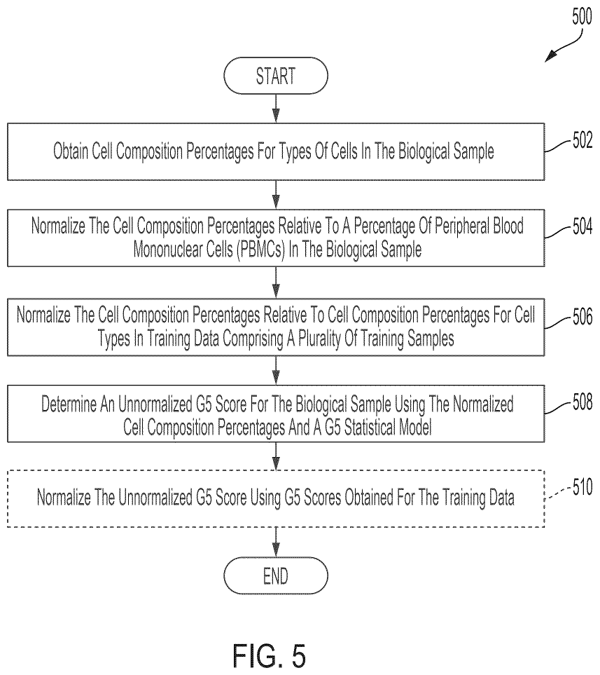

FIG. 5 is a flowchart of an illustrative process 500 for determining a G5 signature for a biological sample, according to some embodiments of the technology described herein, Process 500 may be performed in part or in full by a laptop computer, a desktop computer, one or more servers, in a cloud computing environment, computing device as described herein with respect to FIG. 6 or using any other suitable computing device(s), as aspects of the technology described herein are not limited in this respect.

Process 500 begins at act 502 for obtaining cell composition percentages for types of cells in the biological sample. In some embodiments, act 502 may be performed in any suitable way as described herein. For example, cell composition percentages may be obtained by processing immune cell data obtained for the biological sample. Example techniques for determining cell composition percentages are described herein including at least in the section “Cell Composition Percentages.” In some embodiments, a cell composition percentage may be obtained for peripheral blood mononuclear cells (PBMCs) in the biological sample (e.g., the total percentage of PBMCs of all types, or a sum of percentages of PBMCs of a plurality of types). In some embodiments, a cell composition percentage may be obtained for each of a plurality of immune cell types (e.g. a plurality of types of peripheral blood mononuclear cells) in the biological sample. Additionally, or alternatively, in some embodiments, cell composition percentages may be obtained for at least some (e.g., all) of the cell types listed in Table 6.

Next, at act 504, at least some of the cell composition percentages obtained at act 502 are normalized relative to the cell composition percentage of peripheral blood mononuclear cells (PBMCs) in the biological sample (e.g., the total percentage of PBMCs of all types, or a sum of percentages of PBMCs of a plurality of types). For example, cell composition percentages for cell types listed in Table 6 may be normalized relative to the cell composition percentage of PBMCs (e.g., the total percentage of PBMCs of all types, or a sum of percentages of PBMCs of a plurality of types). Any suitable normalization techniques may be performed relative to the cell composition percentage of PBMCs. For example, the normalizing may include dividing the cell composition percentages by the cell composition percentage of PBMCs (e.g., the total percentage of PBMCs of all types, or a sum of percentages of PBMCs of a plurality of types).

At act 506, the normalized cell composition percentages obtained at act 504 may be normalized relative to cell composition percentages for cell types in training data comprising a plurality of training samples. The training samples may be obtained or may have been previously obtained from one or more healthy subjects (e.g., subjects who do not have, are not suspected of having and/or are not at risk of having cancer) and/or one or more subjects with solid tumors. In some embodiments, the training data includes an indication of an immunoprofile type for the training sample.

In some embodiments, the indication of the immunoprofile type may include an indication of whether the training sample has been classified as G1 type, G2 type, G3 type, G4 type, or G5 type. In some embodiments, the indication includes any suitable indication, as aspects of the technology described herein are not limited in this respect. For example, the indication may be encoded by assigning a value of 1 to samples classified as G5 type and by assigning a value of 0 to samples classified as non-G5 types. Example techniques for determining an immunoprofile type for a subject are described in International Application No. PCT/US2023/080339, published as International Publication No. WO2024/108156 on May 5, 2023.

In some embodiments, the cell composition percentages in the training data includes cell composition percentages of PBMCs in the training samples and/or cell composition percentages for cell types listed in Table 6 in the training samples. In some embodiments, the cell composition percentages in the training data are normalized. For example, the cell composition percentages (e.g., cell composition percentages for cell types listed in Table 6) obtained for a training sample may be normalized relative to the cell composition percentage of PBMCs in the training sample (e.g., the total percentage of PBMCs of all types, or a sum of percentages of PBMCs of a plurality of types).

In some embodiments, the training cell composition percentages may be obtained using any suitable techniques, as aspects of the technology described herein are not limited in this respect. For example, in some embodiments, the cell composition percentages are obtained from a data store (e.g., a public data store). In some embodiments, the cell composition percentages are obtained for the biological samples by processing cell population data and/or RNA expression data obtained for the biological samples. For example, the cell population data and/or RNA expression data may be obtained from a data store (e.g., a public data store), by processing biological samples from one or more subjects, or obtained in any other suitable manner, as aspects of the technology described herein are not limited in this respect.

In some embodiments, the normalizing is performed using any suitable normalization technique, as aspects of the technology described herein is not limited in this respect. For example, in some embodiments, the normalizing is performed using quantiles of the distribution of cell composition percentages (e.g., normalized cell composition percentages) in the training data. For example, the normalizing may be performed using at least two quantiles of the distribution of cell composition percentages in the training data. The quantile(s) may be any suitable quantile(s) as aspects of the technology described herein are not limited in this respect. For example, a first quantile (e.g., q1) may be the 0.01 quantile, the 0.02 quantile, the 0.03 quantile, the 0.04 quantile, the 0.05 quantile, any quantile between the 0.01 quantile and the 0.1 quantile, or any other suitable quantile as aspects of the technology described herein are not limited in this respect. Additionally, or alternatively, the second quantile (e.g., q2) may be the 0.90 quantile, the 0.95 quantile, the 0.96 quantile, the 0.97 quantile, the 0.98 quantile, the 0.99 quantile, any quantile between the 0.90 quantile and the 0.99 quantile, or any other suitable quantile as aspects of the technology described herein are not limited in this respect. As one nonlimiting example, the normalizing may be performed using the 0.02 quantile and the 0.98 quantile of the training data.

Normalized cell composition percentage (CCPN) may be computed according to:

$${CCP}_{N} = ( CCP - q 1 ) / ( q 2 - q 1 )$$

However, it should be appreciated that the cell composition percentages may be normalized according to any other suitable techniques, as aspects of the technology described herein are not limited in this respect.

In some embodiments, the normalized cell composition percentages may be adjusted. For example, normalized cell composition percentages greater than a predetermined value (e.g., one) may be replaced with a value of one. Additionally, or alternatively, normalized cell composition percentages less than a predetermined value (e.g., zero) may be replaced with a value of zero.

At act 508, an unnormalized G5 signature is determined for the biological sample using the normalized cell composition percentages and a G5 signature statistical model. In some embodiments, this includes determining a combination (e.g., linear or non-linear) of the normalized cell composition percentages. In some embodiments, determining the combination of normalized cell composition percentages includes using previously determined coefficients to determine a weighted sum of the normalized cell composition percentages, as described herein. The G5 signature statistical model may include any suitable statistical model. A suitable statistical model may be any multivariate model that can be used to classify an observation comprising values for a plurality of cell composition percentages. For example, the statistical model may be a generalized linear model (e.g., a linear regression model, a logistic regression model, a probit regression model, an Elastic Net regression model, etc.). It should be appreciated that, in some embodiments, the statistical model may not be a generalized linear model and may be a different type of statistical model such as, for example, a random forest regression model, a neural network, a support vector machine, a Gaussian mixture model, a hierarchical Bayesian model, and/or any other suitable statistical model, as aspects of the technology described herein are not limited to using generalized linear models for determining the unnormalized G5 signature.

In some embodiments, the statistical model is trained by determining coefficients for the normalized cell composition percentages, and using the coefficients to determine a weighted sum of the normalized cell composition percentages. For example, coefficients may be estimated based on training data (e.g., the training set of cell composition percentages). Example coefficients are listed for cell types in Table 6. In some embodiments, the training data includes, for each training sample, the cell composition percentages and a known immunoprofile type. In some embodiments, indications of known immunoprofile types (e.g., encoded as 0 and 1) are used as target values for the regression. In some embodiments, the coefficients are estimated by performing a regression analysis on the training data.

At act 512, the unnormalized G5 signatures (e.g., for the biological sample and/or for the training samples) may optionally be normalized. For example, the unnormalized G5 signatures may be normalized to range of values having any suitable upper bound and any suitable lower bound, as aspects of the technology described herein are not limited in this respect. For example, the lower bound may be a value between 0.01 and 0.50, between 0.02 and 0.45, between 0.03 and 0.40, between 0.04 and 0.35, between 0.05 and 0.30, between 0.06 and 0.25, between 0.07 and 0.20, between 0.08 and 0.15, or a value in any other suitable range as aspects of the technology described herein are not limited in this respect. Additionally, or alternatively, the upper bound may be a value between 5 and 15, between 6 and 14, between 7 and 13, between 8 and 12, between 9 and 11, or a value in any other suitable range of values as aspects of the technology described herein are not limited in this respect.

In some embodiments, the normalizing may be performed using any suitable normalization technique, as aspects of the technology described herein are not limited in this respect. In some embodiments, the normalizing is performed using quantiles of the G5 signatures determined for training samples. For example, the normalizing may be performed using at least two quantiles of the distribution of G5 signatures determined for the training samples. The quantile(s) may be any suitable quantile(s) as aspects of the technology described herein are not limited in this respect. For example, a first quantile (e.g., qp1) may be the 0.01 quantile, the 0.02 quantile, the 0.03 quantile, the 0.04 quantile, the 0.05 quantile, any quantile between the 0.01 quantile and the 0.1 quantile, or any other suitable quantile as aspects of the technology described herein are not limited in this respect. Additionally, or alternatively, the second quantile (e.g., qp2) may be the 0.90 quantile, the 0.95 quantile, the 0.96 quantile, the 0.97 quantile, the 0.98 quantile, the 0.99 quantile, any quantile between the 0.90 quantile and the 0.99 quantile, or any other suitable quantile as aspects of the technology described herein are not limited in this respect. As one nonlimiting example, the normalizing may be performed using the 0.01 quantile and the 0.99 quantile of the distribution of G5 signatures determined for the training samples.

A normalized G5 signature (G5_{N}) may be computed according to:

$${G}_{5} = 9.9 * \frac{G 5 - qp 1}{qp 2 - qp 1} + \text{.1}$$

However, it should be appreciated that the cell composition percentages may be normalized according to any other suitable techniques, as aspects of the technology described herein are not limited in this respect.

TABLE 6
Example cell types and statistical model coefficients.

| | G5 Suppressive |
|---|---|
| Population | coefficient |
| CD8 Naïve T cells | −0.029326534 |
| CD4 Naïve T cells | −0.027904185 |
| CD4 Naïve Tregs | −0.025704646 |
| CD8 T cells | −0.054050754 |
| CD4 T cells | −0.070096498 |
| Non-switched Memory IgM B cells | −0.046632291 |
| gdT Vdelta2+ | −0.011357972 |
| Plasmacytoid Dendritic cells | −0.056114463 |
| Naïve B cells | −0.016298942 |
| Dendritic cells | −0.096821076 |
| CD4 Tregs | −0.058998861 |
| Class-switched Memory | −0.007362301 |
| CD8 Effector Memory | −0.037154436 |
| CD4 TEMRA | 0.024215902 |
| Eosinophils | −0.051737814 |
| NKT cells | −0.008193872 |
| Basophils | −0.032767623 |
| CD8 Transitional Memory | −0.036938607 |
| CD8 TEMRA | −0.02080568 |
| Immature NK cells | −0.003313088 |
| CD4 Effector Memory | −0.019019483 |
| CD39 CD4 Tregs | −0.032916215 |
| Neutrophils | 0.093765225 |
| TIGIT+ PD1+ CD8 T cells | −0.037551525 |
| HLA-DR-low Monocytes | 0.136060987 |
| CD8 Memory T cells | −0.045640349 |
| Non-classical Monocytes | 0.009235295 |
| CD4 Transitional Memory | −0.054536799 |
| Granulocytes | 0.091932557 |
| Classical Monocytes | 0.062730277 |
| Mature NK cells | −0.022748525 |
| CD8 Central Memory | −0.031454879 |
| CD4 Memory T helpers | −0.059224879 |
| CD4 Central Memory | −0.061408384 |

### Computer Implementation

An illustrative implementation of a computer system 600 that may be used in connection with any of the embodiments of the technology described herein (e.g., such as the processes of FIGS. 3A-3C) is shown in FIG. 6. The computer system 600 includes one or more processors 610 and one or more articles of manufacture that comprise non-transitory computer-readable storage media (e.g., memory 620 and one or more non-volatile storage media 630). The processor 610 may control writing data to and reading data from the memory 620 and the non-volatile storage media 630 in any suitable manner, as the aspects of the technology described herein are not limited to any particular techniques for writing or reading data. To perform any of the functionality described herein, the processor 610 may execute one or more processor-executable instructions stored in one or more non-transitory computer-readable storage media (e.g., the memory 620), which may serve as non-transitory computer-readable storage media storing processor-executable instructions for execution by the processor 610.

Computing system 600 may include a network input/output (I/O) interface 640 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing system 600 may also include one or more user I/O interfaces 650, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationships between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

### Examples

The following examples demonstrate the performance of embodiments of the technology developed by the inventors.

### A. Example 1

Example 1 demonstrates the performance of machine learning models trained to predict the likelihood that a subject will experience a severe immune-related adverse event (irAE) in response to administration of an ICI therapy, and a machine learning model trained to predict the likelihood that a subject will develop inflammatory bowel disease (IBD) in response to administration of an ICI therapy.

### 1. Datasets

Multiple cohorts were used to train and test the machine learning models described in these examples. The cohorts are summarized in Table 7, and described in more detail below.

TABLE 7
Training and testing datasets.

| | | | Total irAE / | |
|---|---|---|---|---|
| Model | Cohort | Number | Severe irAE | Diagnosis |
| irAE, IBD | Training: RADIOHEAD set | 702 | 174/44 | Pancancer |
| | Testing: RADIOHEAD set | 157 | 39/11 | Pancancer |
| irAE | Testing: MGH set | 47 | 10/10 | Melanoma |
| | Testing: Open-source set 1 | 46 | 34/15 | Melanoma |
| | Testing: Open-source set 2 | 48 | 11/6 | Pancancer |
| IBD | Training: Open-source set 3 | 1792 + 1602 | —/— | IBD + Healthy |
| | Training: Open-source set 3 | 448 + 396 | —/— | IBD + Healthy |

### RADIOHEAD Dataset

The RADIOHEAD dataset includes pre-treatment blood samples (PBMCs) from the pancancer RADIOHEAD cohort (n=859) were treated with various immune checkpoint inhibitor (ICI) therapies, including anti-PD-1, anti-PD-L1, anti-PD-1+anti-CTLA-4, anti-PD-1+chemotherapy, and others. The incidence of severe irAEs in the RADIOHEAD dataset is approximately 6% (55/859). Severe irAEs include grade 3 and 4 irAEs. The RADIOHEAD dataset is described by Quandt, Z., et al. (“Associations between immune checkpoint inhibitor response, immune-related adverse events, and steroid use in RADIOHEAD: a prospective pan-tumor cohort study.” Journal for Immunotherapy of Cancer 13.5 (2025): e011545), which is incorporated by reference herein in its entirety.

The RADIOHEAD cohort was divided into training (Table 8) and test (Table 9) subsets in an 80:20 ratio. The train-test split was performed using the train_test_split function from Scikit-learn, with stratification based on a constructed variable encoding the maximum irAE grade (grade 4) and name.

TABLE 8
Patient characteristics for RADIOHEAD training set.

| Category | All patients, N (%) |
|---|---|
| Patients, (N) | 702 |
| Age, (median, range) | 69.0 (25-89) |
| Sex, M/F | 395 (56.3)/307 (43.7) |
| Therapy: | |
| Anti-PD-1 | 337 (48.0) |
| Anti-PD-1 + Chemotherapy | 116 (16.5) |
| Anti-PDL1 | 100 (14.2) |
| Anti-CTLA-4 + Anti-PD-1 | 65 (9.3) |
| Anti-PDL1 + Chemotherapy | 60 (8.6) |
| Other | 24 (3.4) |
| Diagnosis: | |
| Non Small Cell Lung Carcinoma | 280 (39.9) |
| Melanoma | 86 (12.2) |
| Renal Cell Carcinoma | 69 (9.8) |
| Small Cell Lung Carcinoma | 45 (6.4) |
| Urinary Bladder Neoplasm | 39 (5.6) |
| Other | 183 (26.1) |
| Cancer Stage: | |
| iv | 490 (69.8) |
| iii | 170 (24.2) |
| ii | 21 (3.0) |
| i | 15 (2.1) |
| Unknown | 6 (0.9) |
| Metastatic status, Yes/No | 523 (74.5)/179 (25.5) |
| With irAE, Yes/No | 174 (24.8)/528 (75.2) |
| With severe irAE, Yes/No | 44 (6.3)/658 (93.7) |

TABLE 9
Patient characteristics for RADIOHEAD testing set.

| Category | All patients, N (%) |
|---|---|
| Patients, (N) | 157 |
| Age, (median, range) | 68.0 (30-89) |
| Sex, M/F | 85 (54.1)/72 (45.9) |
| Therapy: | |
| Anti-PD-1 | 86 (54.8) |
| Anti-PDL1 | 27 (17.2) |
| Anti-PD-1 + Chemotherapy | 21 (13.4) |
| Anti-CTLA-4 + Anti-PD-1 | 11 (7.0) |
| Anti-PDL1 + Chemotherapy | 11 (7.0) |
| Other | 1 (0.6) |
| Diagnosis: | |
| Non Small Cell Lung Carcinoma | 71 (45.2) |
| Melanoma | 20 (12.7) |
| Urinary Bladder Neoplasm | 13 (8.3) |
| Renal Cell Carcinoma | 11 (7.0) |
| Small Cell Lung Carcinoma | 10 (6.4) |
| Other | 32 (20.4) |
| Cancer Stage: | |
| iv | 105 (66.9) |
| iii | 40 (25.5) |
| ii | 5 (3.2) |
| i | 6 (3.8) |
| Unknown | 1 (0.6) |
| Metastatic status, Yes/No | 110 (70.1)/47 (29.9) |
| With irAE, Yes/No | 39 (24.8)/118 (75.2) |
| With severe irAE, Yes/No | 11 (7.0)/146 (93.0) |

### MGH Dataset

The MGH dataset includes pre-treatment blood samples (PBMCs) from a melanoma cohort (n=47) treated with either anti-PD-1 (pembrolizumab; n=23) or anti-PD-1 plus anti-CTLA-4 (nivolumab+ipilimumab; n=24) ICI therapy. The severe irAE incidence was approximately 20% (10/47). This cohort was used as an independent test cohort for model validation. Characteristics of patients in the MGH dataset are summarized in Table 10.

TABLE 10
Patient characteristics for MGH testing set.

| Category | All patients, N (%) | |
|---|---|---|
| Patients, (N) | 47 | |
| Age, (median, range) | 67.0 | (38-89) |
| Sex, M/F | 27 (57.4)/20 (42.6) | |
| Therapy, Anti-PD-1/Anti-CTLA-4 + Anti-PD-1 | 23 (48.9)/24 (51.1) | |
| Diagnosis: | | |
| Cutaneous Melanoma | 29 | (61.7) |
| Melanoma | 12 | (25.5) |
| Mucosal Melanoma | 4 | (8.5) |
| Uveal Melanoma | 2 | (4.3) |
| Cancer Stage: | | |
| iv | 41 | (87.2) |
| iii | 4 | (8.5) |
| ii | 1 | (2.1) |
| Unknown | 1 | (2.1) |
| Metastatic status: | | |
| Yes | 41 | (87.2) |
| No | 5 | (10.6) |
| Unknown | 1 | (2.1) |

| | |
|---|---|
| With irAE, Yes/No | 10 (21.3)/37 (78.7) |
| With severe irAE, Yes/No | 10 (21.3)/37 (78.7) |

### Open-Source Dataset 1

Open-source dataset 1 (GSE186143) includes pre-treatment blood samples (PBMCs) from a melanoma cohort (n=46) treated with either anti-PD-1 (n=23) or anti-PD-1 plus anti-CTLA-4 (n=23) ICI therapy. The severe irAE incidence was approximately 30% (15/46). This cohort was used as an independent test cohort for model validation. Severe irAEs include grade 3+irAEs. Open-source dataset 1 is described by Lozano, A. X., et al. (“T cell characteristics associated with toxicity to immune checkpoint inhibitor in patients with melanoma.” Nature medicine 28.2 (2022): 353-362), which is incorporated by reference herein in its entirety. Characteristics of patients in open-source dataset 1 are summarized in Table 11.

TABLE 11
Patient characteristics for open-source testing set 1.

| Category | All patients, N (%) | |
|---|---|---|
| Patients, (N) | 46 | |
| Age, (median, range) | 65.0 | (20-91) |
| Sex, M/F | 30 (65.2)/16 (34.8) | |
| Therapy, Anti-CTLA-4 + Anti-PD-1/Anti-PD-1 | 23 (50.0)/23 (50.0) | |
| Diagnosis: | | |
| Melanoma | 46 | (100.0) |
| Cancer Stage: | | |
| Unknown | 46 | (100.0) |
| Metastatic status: | | |
| No | 46 | (100.0) |

| | |
|---|---|
| With irAE, Yes/No | 34 (73.9)/12 (26.1) |
| With severe irAE, Yes/No | 15 (32.6)/31 (67.4) |

### Open-Source Dataset 2

Open-source dataset 2 (GSE287540) includes pre-treatment blood samples (PBMCs) from a solid neoplasm cohort (n=48) treated with various ICI therapies, including anti-PD-1 plus anti-CTLA-4 (n=35), anti-PD-1 (n=11), anti-PDL1 (n=1) and anti-PDL1 plus TKI (n=1). The severe irAE incidence was approximately 12% (6/48). This cohort was used as an independent test cohort for model validation. The irAE severity status was determined based on whether the patient received steroid treatment in response to an adverse event occurrence (severe) or not (not severe). This cohort was used as an independent test cohort for model validation. Open-source dataset 2 is described by Ji, C., et al. (“Transcriptomic and proteomic characterization of cell and protein biomarkers of checkpoint inhibitor-induced liver injury.” Cancer Immunology, Immunotherapy 74.6 (2025): 190) which is incorporated by reference herein in its entirety. Characteristics of patients in open-source dataset 2 are summarized in Table 12.

TABLE 12
Patient characteristics for open-source testing set 2.

| Category | All patients, N (%) | |
|---|---|---|
| Patients, (N) | 48 | |
| Age, (median, range) | 58.5 | (30-88) |
| Sex, M/F | 37 (77.1)/11 (22.9) | |
| Therapy: | | |
| Anti-CTLA-4 + Anti-PD-1 | 35 | (72.9) |
| Anti-PD-1 | 11 | (22.9) |
| Anti-PDL1 | 1 | (2.1) |
| Anti-PDL1 + TKI | 1 | (2.1) |
| Diagnosis: | | |
| Cancer patient | 48 | (100.0) |
| Cancer Stage: | | |
| Unknown | 48 | (100.0) |
| Metastatic: | | |
| Unknown | 48 | (100.0) |

| | |
|---|---|
| With irAE, Yes/No | 11 (22.9)/37 (77.1) |
| With severe irAE, Yes/No | 6 (12.5)/42 (87.5) |

### Open-Source Dataset 3

Open-source dataset 3 is a combination of multiple datasets including data from a cohort of patients with autoimmune IBD and patients without autoimmune IBD (healthy). The cohort was evenly split into training and test sets using train_test_split with stratification based on disease status. The training set included 1,792 diseased and 1,602 healthy individuals, while the test set comprised 448 diseased and 396 healthy individuals.

The datasets included in open-source dataset 3 include: GSE121578, E-MTAB-6739, GSE92472, GSE143507, GSE161031, GSE159034, GSE171770, GSE191328, GSE177044, GSE186507, GSE117875, GSE69446, GSE95450, GSE99816, E-MTAB-5464, GSE184307, GSE156044, GSE115390, GSE158952, GSE171244, GSE199906, GSE224758, GSE192819, GSE57945, GSE93624, GSE81266, GSE233900, GSE261086, GSE243625, GSE230113, GSE157020, GSE174159, GSE137344, GSE83687, GSE228122, GSE164871, GSE66207, GSE134080, GSE97356, GSE164877, GSE193141, GSE198449, GSE54308, PRJNA938007, GSE215067, E-MTAB-10395, GSE192786, GSE151686, GSE215144, GSE139179, GSE235236, GSE123141, GSE172372, GSE112057, and GSE201533

### 2. Clinical Component

A first machine learning model was trained to predict, from clinical data, a likelihood that a subject will experience an irAE in response to administration of an ICI therapy. The first machine learning model is an example implementation of the first machine learning model 122-1 described herein including at least with respect to FIG. 1B and FIG. 1C.

The first machine learning model was trained on a subset of the RADIOHEAD training set, containing 96 samples. Specifically, the first machine learning model was trained on clinical features (e.g., features included in clinical data, such as clinical data 106-1 in FIGS. 1A-1C) for patients in the balanced subset of the RADIOHEAD training set. The features include:
Demographic features: Gender (M/F), Age.
General clinical indicators: Cancer Stage (I-IV), Metastatic status (Yes/No).
Diagnosis: Anaplastic Astrocytoma, Breast Neoplasm, Colorectal Neoplasm, Endometrial Neoplasm, Esophagogastric Junction Carcinoma, Hepatobiliary Neoplasm, Hepatocellular Carcinoma, Melanoma, Merkel Cell Carcinoma, Non-Small Cell Lung Carcinoma, Renal Cell Carcinoma, Small Cell Lung Carcinoma, Squamous Cell Carcinoma of the Head and Neck, Urinary Bladder Neoplasm.
Therapy type: Anti-CTLA-4+Anti-PD-1, Anti-PD-1, Anti-PD-1+Chemotherapy, Anti-PD-1+Other, Anti-PD-L1, Anti-PD-L1+Chemotherapy.

To achieve a balanced subset of the RADIOHEAD training set, the number of non-severe-irAE cases was reduced to approximately 2.5 times the number of severe irAE cases. When selecting which non-irAE samples to keep, the distribution of clinical factors including gender, disease stage, diagnosis, therapy type, and metastatic status was preserved to ensure that the final subset remained representative of the full cohort. The balancing procedure was implemented using pandas operations without relying on any external resampling libraries.

The features were pre-processed by performing normalization and encoding steps. The cancer stage feature was encoded using OrdinalEncoder from sklearn to preserve its ordinality. Categorical features such as therapy, diagnosis, gender, and metastatic status were transformed using standard one-hot encoding via pandas.get_dummies( ). If a categorical variable was missing in a given subset (e.g., the test cohort contained only melanoma samples), all corresponding columns for absent categories were filled with False. If a categorical variable type was completely unavailable for a sample (e.g., metastatic status data missing entirely), the corresponding columns were filled with −1.

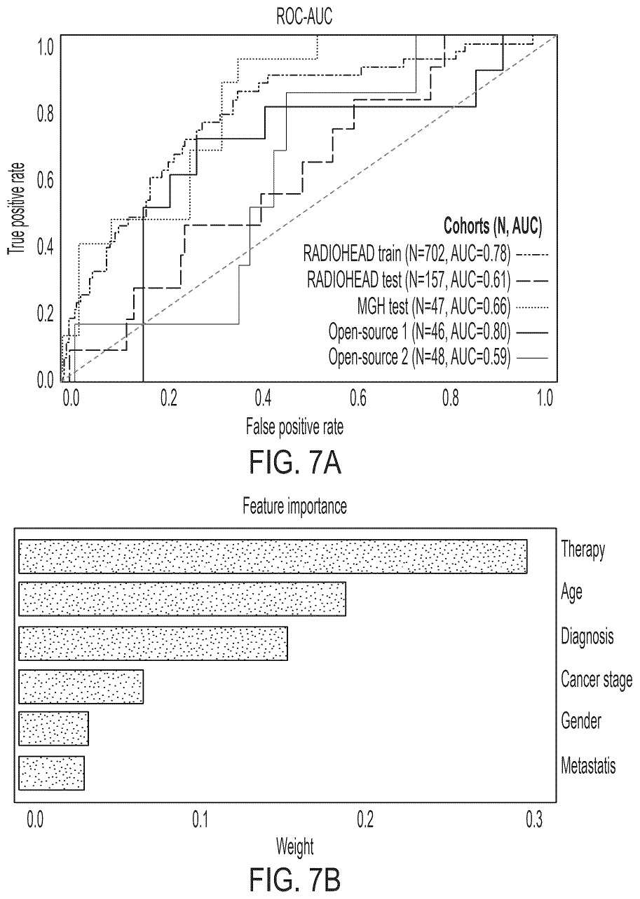

The first machine learning model is a random forest classifier from the sklearn.ensemble package. Hyperparameter optimization was performed using Optuna from optuna.create_study, with the optimization direction set to maximize the cross-validated Receiver Operating Characteristic-Area Under the Curve (ROC-AUC) score. The search was conducted over 100 trials using a 10-fold cross-validation scheme and a fixed random seed of 42 for reproducibility. The Optuna study explored a parameter space defined by the AMLConfiguration framework, which included variations in preprocessing, oversampling, and model selection. Specifically, the configuration considered StandardScaler as the scaler, SMOTE (Synthetic Minority Oversampling Technique) as the oversampling method, and multiple classifier options including logistic regression, random forest, decision tree, and naive Bayes. No additional data transformations were applied. The final model was trained with 107 trees, a maximum depth of 12, a minimum samples per split of 4, a minimum samples per leaf of 3, and class weights set to balanced. Feature importances were then derived from the trained random forest model, allowing for estimation of how strongly each variable contributes to predicting the occurrence of severe irAEs. The resulting feature importances are summarized in Table 13 and illustrated in FIG. 7B.

TABLE 13
First machine learning model feature importances.

| Feature | Importance |
|---|---|
| Age | 0.239702947 |
| Therapy: Anti-CTLA-4 + Anti-PD-1 | 0.146082374 |
| Therapy: Anti-PDL1 | 0.106162414 |
| Cancer Stage | 0.09149034 |
| Diagnosis: Non Small Cell Lung Carcinoma | 0.071580088 |
| Diagnosis: Melanoma | 0.058862057 |
| Therapy: Anti-PD-1 + Chemotherapy | 0.057078707 |
| Diagnosis: Renal Cell Carcinoma | 0.054985118 |
| Therapy: Anti-PD-1 | 0.051499183 |
| Gender: Male | 0.051301039 |
| Metastatic status: Yes | 0.050060946 |
| Therapy: Anti-PDL1 + Chemotherapy | 0.010433416 |
| Diagnosis: Small Cell Lung Carcinoma | 0.010057987 |
| Diagnosis: Squamous Cell Carcinoma of | 0.000703 |
| the Head and Neck | |
| All other features | 0 |

The first machine learning model was trained to predict a likelihood that a subject will experience an immune-related adverse event in response to administration of an ICI therapy. The predicted likelihoods were used to stratify patients into: (i) patients likely to experience severe irAEs and (ii) patients not likely to experience severe irAEs, using a threshold value of 0.49. For example, a patient for whom the predicted likelihood was greater than or equal to 0.49 was identified as a patient likely to experience a severe irAE. The threshold was determined using the RADIOHEAD training cohort using Youden's J statistic. Tables 14-18 summarize characteristics of patients in the training and testing datasets stratified by the presence and absence of severe immune-related adverse events.

The first machine learning model demonstrated robust performance across independent test cohorts, achieving an Area Under the Curve (AUC) of 0.61, 0.66, 0.80, 0.59 in the RADIOHEAD test set and comparable results in validation datasets, as illustrated in FIG. 7A. Table 19 summarizes metrics demonstrating the robust performance of the first machine learning model across testing and validation sets.

TABLE 14
Patient characteristics stratified by the presence or absence of severe
immune-related adverse events in the training RADIOHEAD set.

| | With severe | | Without severe | |
|---|---|---|---|---|
| Category | irAEs, N (%) | | irAEs, N (%) | |
| Patients, (N) | 44 | (6.3) | 658 | (93.7) |
| Age, (median, range) | 65.5 | (30-89) | 69.0 | (25-89) |
| Sex, M/F | 27 (61.4)/17 (38.6) | | 368 (55.9)/290 (44.1) | |
| Therapy: | | | | |
| Anti-PD-1 | 21 | (47.7) | 316 | (48.0) |
| Anti-CTLA-4 + Anti-PD-1 | 13 | (29.5) | 52 | (7.9) |
| Anti-PD-1 + | 5 | (11.4) | 111 | (16.9) |
| Chemotherapy | | | | |
| Anti-PDL1 + | 2 | (4.5) | 58 | (8.8) |
| Chemotherapy | | | | |
| Anti-PDL1 | 2 | (4.5) | 98 | (14.9) |
| Other | 1 | (2.3) | 23 | (3.5) |
| Diagnosis: | | | | |
| Non-Small Cell Lung | 18 | (40.9) | 262 | (39.8) |
| Carcinoma | | | | |
| Melanoma | 9 | (20.5) | 77 | (11.7) |
| Renal Cell Carcinoma | 7 | (15.9) | 62 | (9.4) |
| Small Cell Lung Carcinoma | 2 | (4.5) | 43 | (6.5) |
| Urinary Bladder Neoplasm | 0 | (0.0) | 38 | (5.8) |
| Other | 8 | (18.1) | 176 | (26.7) |
| Cancer stage: | | | | |
| iv | 30 | (68.2) | 460 | (69.9) |
| iii | 10 | (22.7) | 160 | (24.3) |
| ii | 3 | (6.8) | 18 | (2.7) |
| i | 0 | (0.0) | 15 | (2.3) |
| Unknown | 1 | (2.3) | 5 | (0.8) |

| | | |
|---|---|---|
| Metastatic, Yes/No | 31 (70.5)/13 (29.5) | 492 (74.8)/166 (25.2) |

TABLE 15
Patient characteristics stratified by the presence or absence of severe
immune-related adverse events in the RADIOHEAD testing set.

| | With severe | | Without severe | |
|---|---|---|---|---|
| Category | irAEs, N (%) | | irAEs, N (%) | |
| Patients, (N) | 11 | (7.0) | 146 | (93.0) |
| Age, (median, range) | 69.0 | (48-89) | 68.0 | (30-89) |
| Sex, M/F | 6 (54.5)/5 (45.5) | | 79 (54.1)/67 (45.9) | |
| Therapy: | | | | |
| Anti-PD-1 | 5 | (45.4) | 81 | (55.5) |
| Anti-PD-1 + | 3 | (27.3) | 18 | (12.3) |
| Chemotherapy | | | | |
| Anti-CTLA-4 + | 2 | (18.2) | 9 | (6.2) |
| Anti-PD-1 | | | | |
| Anti-PDL1 | 1 | (9.1) | 26 | (17.8) |
| Anti-PDL1 + | 0 | (0.0) | 11 | (7.5) |
| Chemotherapy | | | | |
| Other | 0 | (0.0) | 1 | (0.7) |
| Diagnosis: | | | | |
| Melanoma | 4 | (36.3) | 16 | (11.0) |
| Non-Small Cell Lung | 3 | (27.3) | 68 | (46.6) |
| Carcinoma | | | | |
| Small Cell Lung Carcinoma | 1 | (9.1) | 9 | (6.2) |
| Urinary Bladder Neoplasm | 0 | (0.0) | 12 | (8.2) |
| Renal Cell Carcinoma | 0 | (0.0) | 11 | (7.5) |
| Other | 3 | (27.3) | 30 | (20.5) |
| Cancer stage: | | | | |
| iv | 7 | (63.6) | 98 | (67.1) |
| iii | 3 | (27.3) | 37 | (25.4) |
| ii | 1 | (9.1) | 4 | (2.7) |
| i | 0 | (0.0) | 6 | (4.1) |
| Unknown | 0 | (0.0) | 1 | (0.7) |

| | | |
|---|---|---|
| Metastatic, Yes/No | 9 (81.8)/2 (18.2) | 101 (69.2)/45 (30.8) |

TABLE 16
Patient characteristics stratified by the presence or absence of severe
immune-related adverse events in the training set from MGH.

| | With severe | | Without severe | |
|---|---|---|---|---|
| Category | irAEs, N (%) | | irAEs, N (%) | |
| Patients, (N) | 10 | (21.3) | 37 | (78.7) |
| Age, (median, range) | 68.0 | (50-81) | 63.0 | (38-89) |
| Sex, F/M | 6 (60.0)/4 (40.0) | | 23 (62.2)/14 (37.8) | |
| Therapy, Anti-CTLA-4 + | 5 (50.0)/5 (50.0) | | 19 (51.4)/18 (48.6) | |
| Anti-PD-1/Anti-PD-1 | | | | |
| Diagnosis: | | | | |
| Melanoma | 4 | (40.0) | 8 | (21.6) |
| Cutaneous Melanoma | 3 | (30.0) | 26 | (70.3) |
| Mucosal Melanoma | 2 | (20.0) | 2 | (5.4) |
| Uveal Melanoma | 1 | (10.0) | 1 | (2.7) |
| Cancer stage: | | | 0 | (0.0) |
| iv | 6 | (60.0) | 35 | (94.6) |
| iii | 2 | (20.0) | 2 | (5.4) |
| ii | 1 | (10.0) | 0 | (0.0) |
| Unknown | 1 | (10.0) | 0 | (0.0) |
| Metastatic: | | | | |
| Yes | 6 | (60.0) | 35 | (94.6.0) |
| No | 3 | (30.0) | 2 | (5.4) |
| Unknown | 1 | (10.0) | 0 | (0.0) |

TABLE 17
Patient characteristics stratified by the presence
or absence of severe immune-related adverse events
in the open-source dataset 1 (GSE186143).

| | With severe | | Without severe | |
|---|---|---|---|---|
| Category | irAEs, N (%) | | irAEs, N (%) | |
| Patients, (N) | 15 | (32.6) | 31 | (67.4) |
| Age, (median, range) | 69.0 | (35-87) | 65.0 | (20-91) |
| Sex, M/F | 11 (73.3)/4 (26.7) | | 19 (61.3)/12 (38.7) | |
| Therapy, Anti-CTLA-4 + | 13 (86.7)/2 (13.3) | | 10 (32.3)/21 (67.7) | |
| Anti-PD-1/Anti-PD-1 | | | | |
| Diagnosis: | | | | |
| Melanoma | 15 | (100.0) | 31 | (100.0) |
| Metastatic: | | | | |
| No | 15 | (100.0) | 31 | (100.0) |

TABLE 18
Patient characteristics stratified by the presence
or absence of severe immune-related adverse events
in the open-source dataset 2 (GSE287540).

| | With severe | | Without severe | |
|---|---|---|---|---|
| Category | irAEs, N (%) | | irAEs, N (%) | |
| Patients, (N) | 6 | (12.5) | 42 | (87.5) |
| Age, (median, range) | 63.5 | (44-72) | 58.0 | (30-88) |
| Sex, M/F | 5 (83.3)/1 (16.7) | | 32 (76.2)/10 (23.8) | |
| Therapy: | | | | |
| Anti-CTLA-4 + Anti-PD-1 | 6 | (100.0) | 29 | (69.0) |
| Anti-PD-1 | 0 | (0.0) | 11 | (26.2) |
| Anti-PDL1 | 0 | (0.0) | 1 | (2.4) |
| Anti-PDL1 + TKI | 0 | (0.0) | 1 | (2.4) |
| Diagnosis: | | | | |
| Cancer patient | 6 | (100.0) | 42 | (100.0) |

TABLE 19
Metrics demonstrating the performance of the first machine
learning model across testing and validation sets.

| | | | Open- | Open- | |
|---|---|---|---|---|---|
| | RADIOHEAD | MGH | source 1 | source 2 | All Test |
| | Test | Test | Test | Test | Sets |
| AUC | 0.610 | 0.66 | 0.80 | 0.59 | 0.73 |
| Precision | 0.08 | 0.22 | 0.32 | 0.13 | 0.18 |
| Recall | 0.45 | 0.80 | 1.00 | 1.00 | 0.81 |
| Specificity | 0.60 | 0.22 | 0.00 | 0.00 | 0.38 |
| F1-Score | 0.14 | 0.34 | 0.49 | 0.22 | 0.29 |
| Fisher p- | 0.76 | 1.00 | 1.00 | 1.00 | 0.02 |
| Value | | | | | |
| Odds Ratio | 1.26 | 1.10 | ∞ | ∞ | 2.55 |

### 3. Transcriptomic Component

A second machine learning model was trained to predict, from sequencing data, a likelihood that a subject will experience an irAE in response to administration of an ICI therapy. The second machine learning model is example implementation of the second machine learning model 122-2 described herein including at least with respect to FIG. 1B and FIG. 1D.

### Features

All features used to train the second machine learning model were calculated based on TPM-normalized bulk RNA-seq data quantified using Kallisto. Kallisto is described by Bray, N. L., et al. (“Near-optimal probabilistic RNA-seq quantification.” Nature biotechnology 34.5 (2016): 525-527), which is incorporated by reference herein in its entirety. Prior to signature calculation, TPM values were renormalized to 18,792 blood-relevant genes.

The features used to train the second machine learning model includes: cell population proportions, a G5 signature, and immune signatures.

The cell population proportions include: (i) a proportion of cDCs to dendritic cells, and (ii) a proportion of memory T cells and T cells. cDCs were identified through differential analysis of the internal training cohort, while memory T cells were selected based on their established role in immune activation and autoimmunity during ICI therapy. The cell population proportions were derived by determining cell composition percentages from bulk RNA-seq data using the Kassandra deconvolution model. Techniques for determining cell composition percentages are described in the section entitled “Cell Composition Percentages.” The Kassandra deconvolution model is described by Zaitsev, A., et al. (“Precise reconstruction of the TME using bulk RNA-seq and a machine learning algorithm trained on artificial transcriptomes.” Cancer Cell 40.8 (2022): 879-894), which is incorporated by reference herein in its entirety.

The G5 signature reflects the activity of myeloid cells in peripheral blood and is associated with immune suppression. Techniques for determining a G5 signature are described in the section entitled “Immunoprofile Type Signatures” and by Dyikanov, D., et al. (“Comprehensive peripheral blood immunoprofiling reveals five immunotypes with immunotherapy response characteristics in patients with cancer.” Cancer Cell 42.5 (2024): 759-779), which is incorporated by reference herein in its entirety.

In addition, a set immune signatures, calculated with ssGSEA, was included to represent specific biological pathways and cellular processes relevant to immune activation and regulation. The immune signatures included: CD4-related signature, antigen specific T-cell activation signature, Treg and T-cell activation signature, LDHB glycolysis signature, Treg signature, irAE-associated T-cell signature, M2 polarization signature, myeloid suppression signature, LDHA glycolysis signature, hypoxia factors signature, autophagy signature, and platelet signature. The genes used to compute each signature are listed in Table 20.

Among them, the Treg and T-cell activation signature, antigen specific T-cell activation signature, Treg signature, M2 polarization signature (reflecting macrophage polarization), hypoxia factors signature, autophagy signature, and platelet signature were selected based on their association with irAE in the training cohort. As some other features represent related cell types, to improve specificity toward regulatory T cells, the Treg signature was refined using internal paired RNA-seq and cytometry data.

The CD4-related signature, irAE-associated T-cell signature, myeloid suppression signature, LDHB glycolysis signature, and LDHA glycolysis signature were developed. The development involved identifying cell types and processes associated with irAE, selecting core genes related to these cell types and processes, adding genes based on their correlation with core genes and their function described in literature.

The TNF signaling-associated signature was developed using datasets of immune cell subtypes, including sorted classical/non-classical monocytes subtypes, each containing at least five samples. For each dataset, the expression of all genes was correlated with a target gene, for example, ETS2, and genes exceeding the 75th percentile for both mean and median correlations were selected. The list was refined by confirming consistent expression and high correlation across monocyte and PBMC datasets and validated for functional relevance through open databases, such as STRING and literature evidence.

TABLE 20
Genes associated with immune signatures.

| Gene Group | Genes |
|---|---|
| LDHB glycolysis | LDHB, DGKA, GCNT4, TBC1D4, ETS1 |
| signature | |
| Treg and T-cell | ABCC1, ARID5B, BCL2, BIRC3, CCND2, CCR4, |
| activation | CD2, CD28, CISH, CTLA4, FAS, FOXP3, |
| signature | GATA3, ICOS, IL12RB2, IL2RA, IL2RB, LTA, |
| | MAF, MAP3K14, OPTN, P2RY10, PIM2, |
| | POU2AF1, RTKN2, SLAMF1, SOCS1, SOCS2, |
| | TIGIT, TRADD, TRAF1, TRAF2 |
| irAE-associated | TNFRSF4, CD28, KLRB1, TNFRSF18, CD40, |
| T-cell | IFNG, TRAT1, EOMES, CD69, CCR8, GZMA, |
| signature | TIGIT, TNFRSF9, ZAP70, TCF7, KLRK1, ICOS, |
| | CD8B, FASLG, CD27, IKZF2, PRF1, GZMB, |
| | LAIR2, GZMK, CCL5, CD5, GZMH, CD8A, |
| | PFKP, CD40LG, KLRD1, TBX21, NKG7, |
| | GNLY, CTLA4, TRAC |
| Treg signature | FOXP3, CTLA4, IL2RA, CCR8, IKZF4, |
| | IKZF2, RTKN2, CCR4, FAS |
| CD4-related | CD28, TCF7, IL2RA, CHMP7, CCR4, CAMK4, |
| signature | S1PR1, DUSP16, MAL, AQP3, CCR7, RASA3, |
| | CD40LG, GATA3, KCNA3, RCAN3, ZC3H12D, |
| | CD6, LRIG1, TRAF1, TRAT1, CD27, TRABD2A, |
| | TESPA1, ICOS, CACNA1I, ITPKB, PIK3C2B, |
| | TNFRSF10A, CD5 |
| Antigen specific | TESPA1, SIRPG, CD3G, SLAMF6, CD27, LCK, |
| T-cell | IKZF3, FCMR, LDLRAP1, LTB, EPB41, LAT, |
| activation | CD3D, PTPRCAP, ADD3, CD2, MAP4K1, SIT1, |
| | ESYT1, UBASH3A, TRAF3IP3, CD3E, SAMD3, |
| | THEMIS, LIME1, LY9, GRAP, SKAP1, TCF7, |
| | ITM2A, KLRG1 |
| Hypoxia factors | FUT11, NDRG1, EPAS1, CA9, LDHA, LOX, |
| signature | SLC2A1, P4HA1, CA12, HK2, PDK1, PGK1, |
| | TPI1, ALDOA, PFKFB3 |
| LDHA glycolysis | HAVCR2, PGK1, LDHA, PSMA6, BPGM, PDIA3, |
| signature | PDIA6, PLIN2, SPPL2A, LGALS8, YARS, |
| | HSP90B1, MAGT1, SKIL, GSTO1 |
| Platelet signature | ITGA2B, ITGB3, SELP, MPL, GP1BA, GP1BB, |
| | TUBB1 |
| TNF signaling- | AREG, EREG, LAMB3, PLAU, PTX3 |
| associated signature | |
| Myeloid suppression | TGFB2, IL10, CCL24, CXCL8, S100A12, EBI3, |
| signature | MSR1, PTGS2, SLC11A1, TREM1, PLAUR |
| M2 polarization | TGFB2, TGFB3, IL10, CCL18, IL33, CCL24 |
| signature | |
| Autophagy signature | ATG12, ATG9A, TFEB, RB1CC1, MAP1LC3B, |
| | GABARAPL2, ATG4B, ATG7, GABARAP, |
| | VMP1, ATG14, GABARAPL1, ATG13, NBR1 |

### Training

All features were represented as continuous variables. Prior to model training, all features were scaled using RobustScaler( ) from the sklearn.preprocessing, which normalizes values based on the interquartile range (25th and 75th quantiles), followed by QuantileTransformer(n_quantiles=100) from the sklearn.preprocessing package to map feature distributions to a uniform scale. Missing values were imputed with zeros.

The second machine learning model was trained on the RADIOHEAD training dataset with the target variable representing severe irAE occurrence. Logistic regression was applied with an L2 penalty, the solver was set to “lbfgs”, inverse regularization strength was set to C=0.0132, maximum iterations was set to 1000, and class weights were set to balanced. Hyperparameter optimization was performed using Optuna from optuna.create_study across a combined search space defined by the AMLConfiguration framework. The search space included the regularization parameter C∈[10-3, 102] (log-scaled) and multiple preprocessing configurations involving alternative scalers (StandardScaler, MinMaxScaler, RobustScaler, MaxAbsScaler) and data transformers (PowerTransformer, QuantileTransformer). The final configuration, selected through cross-validation, used RobustScaler and QuantileTransformer, resulting in the parameters described above. No random seed was applied during the tuning process. The final model was trained with an intercept value of 0.037866, representing the log-odds of severe irAE occurrence when all predictor variables are zero. Model coefficients serve as estimates of the strength and direction of association between each transcriptomic feature and the occurrence of adverse events.

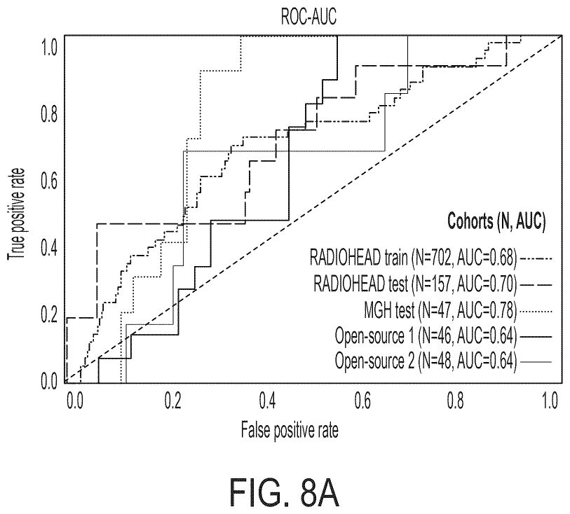

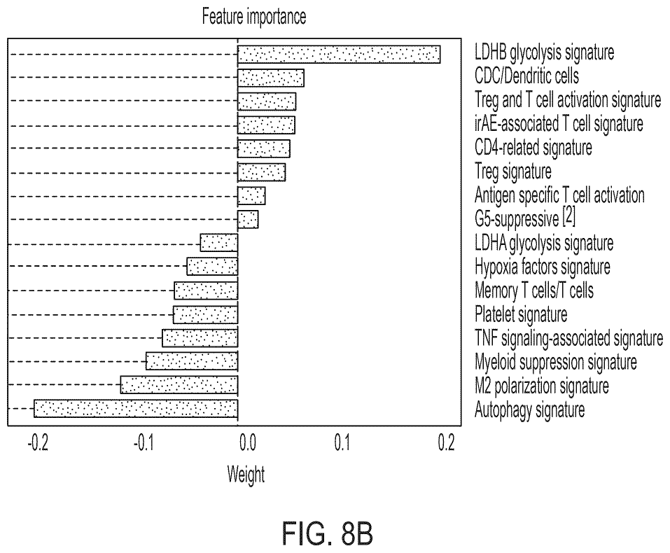

Feature coefficients are summarized in Table 21 and illustrated in FIG. 8B. Feature contribution analysis revealed that upregulation of genes related to T-cell activation and antigen presentation was associated with higher irAE probability, whereas signatures of myeloid suppression and regulatory T-cell activity showed protective effects.

TABLE 21
Feature coefficients.

| Feature | Importance |
|---|---|
| CDC/Dendritic cells | 0.233016 |
| LDHB glycolysis signature | 0.0758 |
| Treg and T-cell activation signature | 0.066659 |
| irAE-associated T-cell signature | 0.0656 |
| Treg signature | 0.060637 |
| CD4-related signature | 0.054257 |
| G5-suppressive | 0.031214 |
| Antigen specific T-cell activation | 0.022838 |
| Hypoxia factors signature | −0.04319 |
| LDHA glycolysis signature | −0.05717 |
| Platelet signature | −0.07325 |
| Memory T-cells/T-cells | −0.07386 |
| TNF signaling-associated signature | −0.08612 |
| Myeloid suppression signature | −0.10398 |
| M2 polarization signature | −0.13448 |
| Autophagy signature | −0.2336 |

### Results

The second machine learning model was trained to predict a likelihood that a subject will experience an immune-related adverse event in response to administration of an ICI therapy. The predicted likelihoods were used to stratify patients into: (i) patients likely to experience severe irAEs and (ii) patients not likely to experience severe irAEs. The threshold used for stratifying patients was 0.50. For example, a patient for whom the predicted likelihood was greater than or equal to 0.50 was identified as a patient likely to experience a severe irAE. The threshold was determined using the RADIOHEAD training cohort using Youden's J statistic.

The second machine learning model reached an AUC of 0.70, 0.78, 0.64 and 0.64 in the independent test sets, demonstrating consistent predictive capacity in multiple external cohorts, as illustrated in FIG. 8A. Table 22 summarizes metrics demonstrating the robust performance of the second machine learning model across testing and validation sets.

TABLE 22
Metrics demonstrating the performance of the second machine
learning model across testing and validation sets.

| | | | Open- | Open- | |
|---|---|---|---|---|---|
| | RADIOHEAD | MGH | source 1 | source 2 | All Test |
| | Test | Test | Test | Test | Sets |
| AUC | 0.70 | 0.78 | 0.64 | 0.64 | 0.72 |
| Precision | 0.10 | 0.38 | 0.39 | 0.10 | 0.22 |
| Recall | 0.55 | 1.00 | 0.60 | 0.17 | 0.62 |
| Specificity | 0.64 | 0.57 | 0.55 | 0.79 | 0.64 |
| F1-Score | 0.17 | 0.56 | 0.47 | 0.13 | 0.33 |
| Fisher p- | 0.33 | 1.1*10⁻³ | 0.53 | 1.00 | 2.0*10⁻³ |
| Value | | | | | |
| Odds Ratio | 2.11 | ∞ | 1.82 | 0.73 | 2.90 |

### 4. xCR Component

A third machine learning model was trained to predict, from TCR and BCR receptor metrics, a likelihood that a subject will experience an irAE in response to administration of an ICI therapy. The third machine learning model is an example implementation of the third machine learning model 122-3 described herein including at least with respect to FIG. 1B and FIG. 1E.

### Features

The third machine learning model was trained using three features: BCR mean Shannon index, TCR mean Shannon index, and total IgHV4-34 proportion. For BCR, the mean Shannon index was computed across heavy, kappa, and lambda chains. For TCR, the mean Shannon index was computed across alpha and beta chains. The total IgHV4-34 proportion represents the cumulative fraction of clonotypes within IgHV4-34 among all heavy chain sequences. IgHV4-34 was selected due to its association as with autoimmune disease as described by Bashford-Rogers, R. J., et al. (“Antibody repertoire analysis in polygenic autoimmune diseases,” Immunology 155.1 (2018): 3-17), which is incorporated by reference herein in its entirety.

Feature scaling and normalization were performed using RobustScaler( ), followed by QuantileTransformer(n_quantiles=100) to map feature distributions to a uniform scale. Missing feature values were filled with 0 prior to model training.

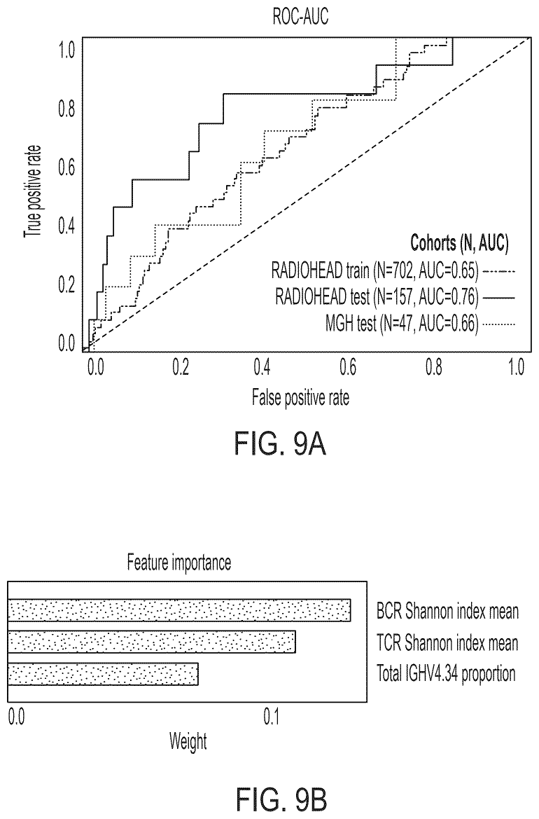

Feature coefficients are summarized in Table 23 and illustrated in FIG. 9B.

TABLE 23
Feature coefficients.

| Feature | Importance |
|---|---|
| BCR Shannon Index Mean | 0.167447 |
| TCR Shannon Index Mean | 0.140364 |
| Total IGHV4.34 Proportion | 0.093369 |

### Training

The third machine learning model was trained on the RADIOHEAD training cohort, with the target variable representing severe irAE occurrence, using a logistic regression classifier with an L2 penalty, solver set to “Ibfgs”, inverse regularization strength set to C=0.0132, maximum iterations of 1000, class weights set to balanced, and an intercept of −0.214683. Hyperparameter optimization was conducted using Optuna from optuna.create_study, following an identical procedure to the transcriptomic model, to select the optimal combination of preprocessing and model parameters.

### Results

The third machine learning model was trained to predict a likelihood that a subject will experience an immune-related adverse event in response to administration of an ICI therapy. The predicted likelihoods were used to stratify patients into: (i) patients likely to experience severe irAEs and (ii) patients not likely to experience severe irAEs. The threshold for stratifying patients was 0.49. For example, a patient for whom the predicted likelihood was greater than or equal to 0.49 was identified as a patient likely to experience a severe irAE. The threshold was determined using the RADIOHEAD training cohort using Youden's J statistic.

The third machine learning model achieved AUCs 0.76 and 0.66 across test cohorts, supporting its biological relevance and complementarity to other data modalities, as illustrated in FIG. 9A. Table 24 summarizes metrics demonstrating the robust performance of the third machine learning model across testing and validation sets.

TABLE 24
Metrics demonstrating the performance of the third machine
learning model across testing and validation sets.

| | RADIOHEAD | | All Test |
|---|---|---|---|
| | Test | MGH Test | Sets |
| AUC | 0.76 | 0.66 | 0.75 |
| Precision | 0.09 | 0.26 | 0.13 |
| Recall | 0.81 | 0.90 | 0.86 |
| Specificity | 0.37 | 0.30 | 0.36 |
| F1-Score | 0.16 | 0.40 | 0.23 |
| Fisher p- | 0.33 | 0.41 | 0.05 |
| Value | | | |
| Odds Ratio | 2.64 | 3.81 | 3.31 |

### 5. Meta Model

A fourth machine learning model was trained to integrate the outputs of the first (clinical), second (transcriptomic), and third (xCR) models and output a unified immune-related adverse event risk score representing the likelihood that the subject will experience an immune-related adverse event in response to administration of an ICI therapy. The fourth machine learning model is an example implementation of the fourth machine learning model 126 described herein including at least with respect to FIG. 1B.

The fourth machine learning model takes as input the predicted probabilities (predict_proba) generated by the base models, in the following order: (1) xCR (third machine learning model), (2) transcriptomic (second machine learning model), and (3) clinical (first machine learning model). The model was trained on the RADIOHEAD training cohort, with the target variable representing severe irAE occurrence. No additional feature preprocessing was applied, as the inputs are already probability scores from the base models. Hyperparameter optimization was performed using GridSearchCV from sklearn.model_selection to select the optimal logistic regression parameters. The fourth machine learning model uses a logistic regression classifier with an L2 penalty, solver set to “lbfgs”, inverse regularization strength C=1.0, maximum iterations of 1000, class weights set to None (default), and an intercept of −1.287521.

To mitigate potential batch effects between cohorts, the predicted scores were standardized: for each cohort, the median and median absolute deviation (MAD) scaling were applied. Subsequently, Min-Max scaling (MinMaxScaler from the sklearn.preprocessing library) was employed to rescale all standardized values to a range of 0 to 1.

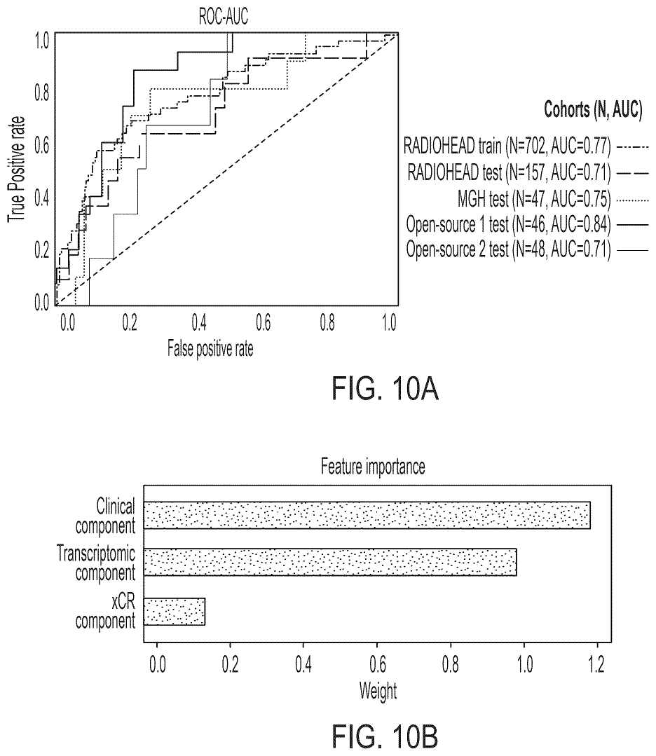

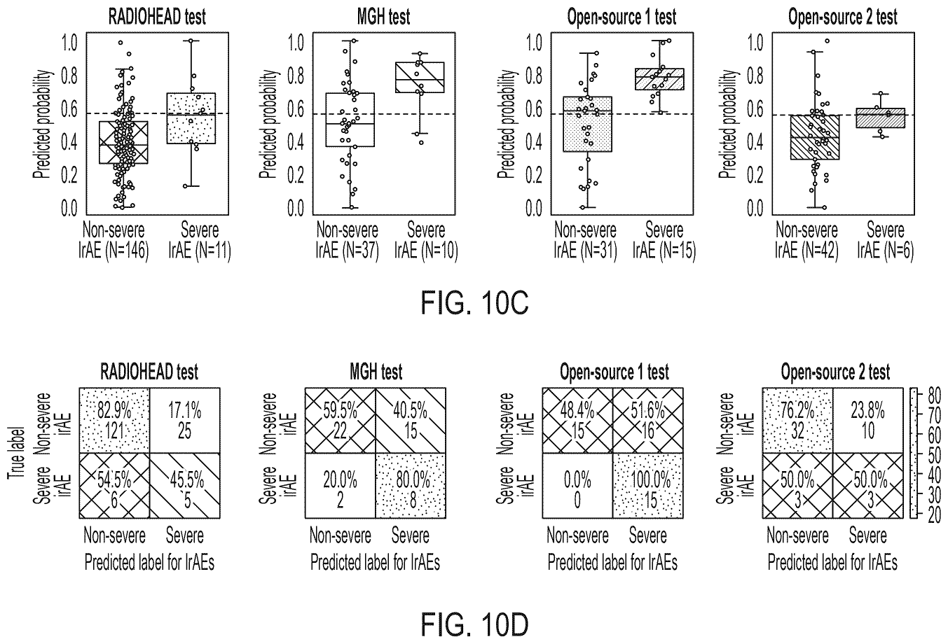

The trained fourth machine learning model outputs a single aggregated probability score ranging from 0 to 1, combining molecular and clinical components to provide a more robust and interpretable estimate of severe irAE occurrence probability. A value closer to 1 indicates a higher predicted likelihood of a severe irAE, while a value closer to 0 indicates a lower likelihood. Feature importances were derived from the trained model. For each feature, two metrics are reported in Table 25: Importance, representing the model coefficient, which reflects the direction and strength of the effect; and Relative Importance (%), representing the normalized contribution of each feature relative to all features, scaled so that the sum of all features equals 100%. Feature importance is illustrated in FIG. 10B.

Feature importance analysis indicated that transcriptomic and repertoire-derived components contributed most strongly to the model's output, while clinical features added stability and generalizability across cohorts

TABLE 25
Feature importances.

| Feature | Importance | Relative Importance (%) |
|---|---|---|
| xcr_predict_proba | 0.151900 | 6.645366 |
| transcriptomic_predict_proba | 0.975253 | 42.665566 |
| clinical_predict_proba | 1.158655 | 50.689068 |

### Results

The fourth machine learning model was trained to predict a likelihood that a subject will experience an immune-related adverse event in response to administration of an ICI therapy. The predicted likelihoods were used to stratify patients into: (i) patients likely to experience severe irAEs and (ii) patients not likely to experience severe irAEs. The threshold for stratifying patients was 0.57. For example, a patient for whom the predicted likelihood was greater than or equal to 0.57 was identified as a patient likely to experience a severe irAE. The threshold was determined using the RADIOHEAD training cohort using Youden's J statistic.

The fourth machine learning model achieved an AUC of 0.71 on the RADIOHEAD test set, 0.75 on the MGH validation cohort, 0.84 on open-source testing set 1, and 0.71 on open-source testing set 2, as shown in FIG. 10A. The classifier effectively distinguished patients with severe irAEs from others across all test datasets (FIG. 10C) and demonstrated balanced sensitivity and specificity as illustrated by the confusion matrix (FIG. 10D). Table 26 summarizes metrics demonstrating the robust performance of the third machine learning model across testing and validation sets.

TABLE 26
Metrics demonstrating the performance of the fourth machine
learning model across testing and validation sets.

| | | | Open- | Open- | |
|---|---|---|---|---|---|
| | RADIOHEAD | MGH | source 1 | source 2 | All Test |
| | Test | Test | Test | Test | Sets |
| AUC | 0.71 | 0.75 | 0.84 | 0.71 | 0.80 |
| Precision | 0.17 | 0.35 | 0.48 | 0.23 | 0.35 |
| Recall | 0.45 | 0.80 | 1.00 | 0.50 | 0.74 |
| Specificity | 0.83 | 0.59 | 0.48 | 0.76 | 0.77 |
| F1-score | 0.24 | 0.48 | 0.65 | 0.32 | 0.47 |
| Fisher p-value | 0.04 | 0.04 | 6.8e−04 | 0.32 | 2*10⁻¹⁰ |
| Odds ratio | 4.03 | 5.87 | ∞ | 3.20 | 9.62 |

### 6. Summary

Table 27 shows that the fourth machine learning model (meta-model), which combines the outputs of the first, second, and third machine learning models (clinical, transcriptomic, and xCR), represents an improvement over the individual machine learning models in predicting whether a subject will experience an immune-related adverse event in response to the administration of an ICI therapy.

TABLE 27
Metrics demonstrating the performance of the first, second, third, and
fourth machine learning models across testing and validation sets.

| | | | Open- | Open- | |
|---|---|---|---|---|---|
| | RADIOHEAD | MGH | source 1 | source 2 | All Test |
| | Test | Test | Test | Test | sets |
| Clinical component | 0.61 | 0.66 | 0.80 | 0.59 | 0.73 |
| Transcriptomic | 0.70 | 0.78 | 0.64 | 0.64 | 0.72 |
| component | | | | | |
| xCR component | 0.76 | 0.66 | — | — | 0.75 |
| Meta model | 0.71 | 0.75 | 0.84 | 0.71 | 0.80 |

### 7. IBD Prediction Model

A machine learning model was trained to predict, from sequencing data, a likelihood that a subject will develop IBD. The machine learning model is an example implementation of the IBD prediction model 172 described herein including at least with respect to FIG. 1F.

### Features

The machine learning model was trained using features associated with human leukocyte antigen (HLA) alleles. The HLA alleles include (a) unique alleles identified in both diseased and healthy cohorts, as well as (b) significant alleles reported in the literature.

First, HLA alleles were identified for diseased and healthy cohorts. For the diseased cohort, open-source RNA-Seq autoimmune datasets were used to identify unique patients based on SNP (STAR) data. This cohort included 1,792 patients with diarrhea or colitis. The healthy cohort consisted of 1,602 individuals from open-source RNA-Seq healthy datasets. Samples included not only PBMC and whole blood, but also sorted immune cells and tissue samples such as ileum, colon, and mucosa, ensuring broader representation of immune-related HLA diversity. Subsequently, for each locus, sub-cohorts were formed within both the diseased and healthy cohorts, depending on locus typing. Locus typing was performed using the arcasHLA tool, which is described by Orenbuch, R., et al. (“arcasHLA: high-resolution HLA typing from RNAseq.” Bioinformatics 36.1 (2020): 33-40), which is incorporated by reference herein in its entirety. Approximately 75% of the samples from each cohort (healthy and autoimmune) were used to identify significant alleles through locus-level statistical testing, while the remaining 25% were reserved for an unbiased model training set.

After identifying unique alleles, statistical analysis was performed. The odds ratio and p-value were calculated using the Barnard test, followed by adjustment of the p-values using the FDR correction method (Benjamini-Hochberg). Table 28 summarizes the results of the statistical analysis. Based on this analysis, a list of unique disease risk or protective alleles for all loci was compiled.

TABLE 28
Results of statistical analysis used to identify HLA alleles.

| | | Disease, | Disease, | | Healthy, | Healthy, | | | Adjusted P- |
|---|---|---|---|---|---|---|---|---|---|
| Allele | Disease | (N) | (%) | Healthy | (N) | (%) | Odds Ratio | P-value | value |
| DRB1*15:04 | 15 | 1290 | 1.16 | 1 | 1177 | 0.08 | 13.82587684 | 0.00059931 | 0.010587819 |
| DRB1*07:34 | 16 | 1290 | 1.24 | 2 | 1177 | 0.17 | 7.373900957 | 0.001517576 | 0.020107877 |
| C*06:201 | 26 | 1340 | 1.94 | 1 | 1185 | 0.08 | 23.41130198 | 1.41E−06 | 0.000333826 |
| DRB1*11:321 | 16 | 1290 | 1.24 | 1 | 1177 | 0.08 | 14.75908084 | 0.000323778 | 0.008139101 |
| DQB1*06:395 | 15 | 1261 | 1.19 | 1 | 1135 | 0.09 | 13.64218736 | 0.000631251 | 0.023040646 |
| DRB1*13:327 | 24 | 1290 | 1.86 | 4 | 1177 | 0.34 | 5.556173608 | 0.00038392 | 0.008139101 |
| DRA*01:07 | 15 | 1286 | 1.17 | 1 | 1179 | 0.08 | 13.89294272 | 0.000588944 | 0.002355777 |
| DRA*01:08 | 14 | 1286 | 1.09 | 1 | 1179 | 0.08 | 12.95664805 | 0.001089952 | 0.003269856 |
| DMA*01:05 | 10 | 1268 | 0.79 | 1 | 1122 | 0.09 | 8.905265019 | 0.013149611 | 0.039448834 |
| DQB1*06:352 | 67 | 1261 | 5.31 | 22 | 1135 | 1.94 | 2.837726703 | 1.11E−05 | 0.001620649 |
| DRB1*01:02 | 89 | 1290 | 6.9 | 45 | 1177 | 3.82 | 1.863695737 | 0.000936917 | 0.014187597 |
| DRB3*02:191 | 20 | 735 | 2.72 | 3 | 671 | 0.45 | 6.22213271 | 0.000577012 | 0.00365441 |
| DRB3*02:01 | 63 | 735 | 8.57 | 10 | 671 | 1.49 | 6.190375125 | 4.13E−10 | 7.84E−09 |
| DQB1*03:518 | 27 | 1261 | 2.14 | 4 | 1135 | 0.35 | 6.182950927 | 7.74E−05 | 0.004774909 |
| DRA*01:06 | 12 | 1286 | 0.93 | 2 | 1179 | 0.17 | 5.540124457 | 0.013825537 | 0.033181288 |
| DRB1*04:334 | 37 | 1290 | 2.87 | 9 | 1177 | 0.76 | 3.830428954 | 8.12E−05 | 0.004305228 |
| DRA*01:05 | 156 | 1286 | 12.13 | 42 | 1179 | 3.56 | 3.735491952 | 9.12E−16 | 1.09E−14 |
| C*02:205Q | 44 | 1340 | 3.28 | 11 | 1185 | 0.93 | 3.621860735 | 5.00E−05 | 0.003953232 |
| E*01:13 | 45 | 1340 | 3.36 | 13 | 1191 | 1.09 | 3.147530859 | 0.000146485 | 0.007470758 |
| DMA*01:06 | 33 | 1268 | 2.6 | 12 | 1122 | 1.07 | 2.470799235 | 0.006285872 | 0.028286424 |
| DRB3*02:25 | 73 | 735 | 9.93 | 31 | 671 | 4.62 | 2.275312972 | 0.000142798 | 0.001356581 |
| DRB3*01:108 | 52 | 735 | 7.07 | 26 | 671 | 3.87 | 1.887902042 | 0.010014028 | 0.047566632 |
| DOB*01:04 | 439 | 909 | 48.29 | 306 | 812 | 37.68 | 1.544135575 | 9.25E−06 | 0.00012031 |
| DMB*01:02 | 172 | 1223 | 14.06 | 116 | 1126 | 10.3 | 1.424702706 | 0.005595991 | 0.039171939 |
| DRA*01:02 | 548 | 1286 | 42.61 | 616 | 1179 | 52.25 | 0.678766859 | 1.86E−06 | 1.12E−05 |
| DPB1*04:02 | 163 | 1298 | 12.56 | 217 | 1170 | 18.55 | 0.630821338 | 4.39E−05 | 0.010501378 |
| DMA*01:01 | 1143 | 1268 | 90.14 | 1053 | 1122 | 93.85 | 0.599304724 | 0.000936008 | 0.008424068 |
| DPA1*01:03 | 1033 | 1287 | 80.26 | 1009 | 1172 | 86.09 | 0.657107731 | 0.00013019 | 0.012107665 |
| DRB1*01:01 | 95 | 1290 | 7.36 | 143 | 1177 | 12.15 | 0.574959798 | 7.01E−05 | 0.004305228 |
| C*03:04 | 75 | 1340 | 5.6 | 120 | 1185 | 10.13 | 0.526319196 | 2.58E−05 | 0.003058463 |
| DRB1*04:07 | 13 | 1290 | 1.01 | 31 | 1177 | 2.63 | 0.376477821 | 0.0033352 | 0.039281248 |

Second, HLA alleles were identified from a literature review. Articles were annotated in the HLA Database (HLA_v.2.0) to identify significant alleles. The database of Anthony Nolan Research Institute (hla.alleles.org/alleles/deleted.html) was used to check for an actual list of alleles. For each diagnosis, literature-derived alleles were filtered to retain only those appearing more than once and exhibiting consistent effects, meaning they influence the odds ratio in the same direction. Table 29 lists HLA alleles and the source from which the HLA allele was identified.

TABLE 29
HLA alleles identified in the literature.

| HLA Alleles | Source |
|---|---|
| A*02:01 | PMID: 25373727 |
| B*07:02 | PMID: 25373727 |
| B*08:01 | PMID: 28067912 |
| B*51:01 | PMID: 25559196 |
| B*52:01 | PMID: 25559196 |
| C*07:01 | PMID: 25559196 |
| C*12:02 | PMID: 25559196 |
| DPB1*04:01 | PMID: 25559196 |
| DQB1*05:01 | PMID: 25559196 |
| DQB1*06:01 | PMID: 25559196 |
| DRB1*01:03 | PMID: 25559196 |
| DRB1*03:01 | PMID: 25559196 |
| DRB1*15:02 | PMID: 25559196 |
| DQB1*02:01 | PMID: 25559196 |
| DRB1*04:01 | PMID: 25559196 |

The HLA alleles identified from the healthy cohorts, diseased cohorts, and literature were combined to form the features used for training the machine learning model. The alleles are listed in Table 30. Table 30 indicates risk alleles and protective alleles. The genomic presence of risk alleles raise susceptibility for disease (IBD), and the genomic presence of protective alleles are common in healthy demographics (non-IBD).

### Training

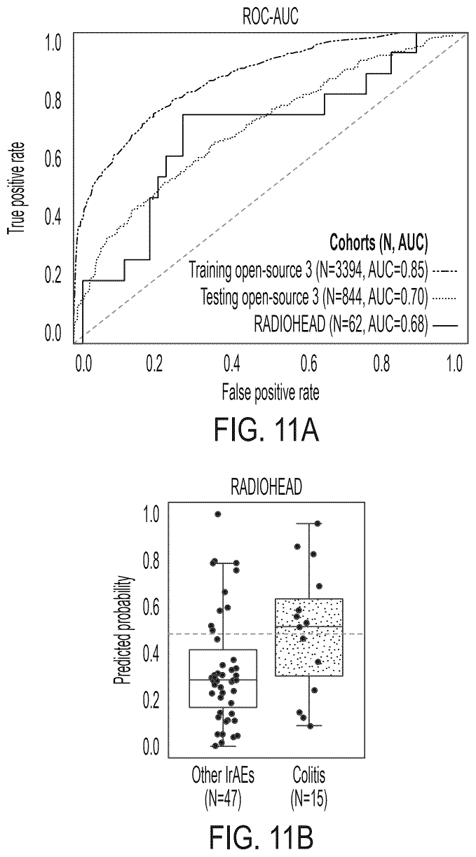

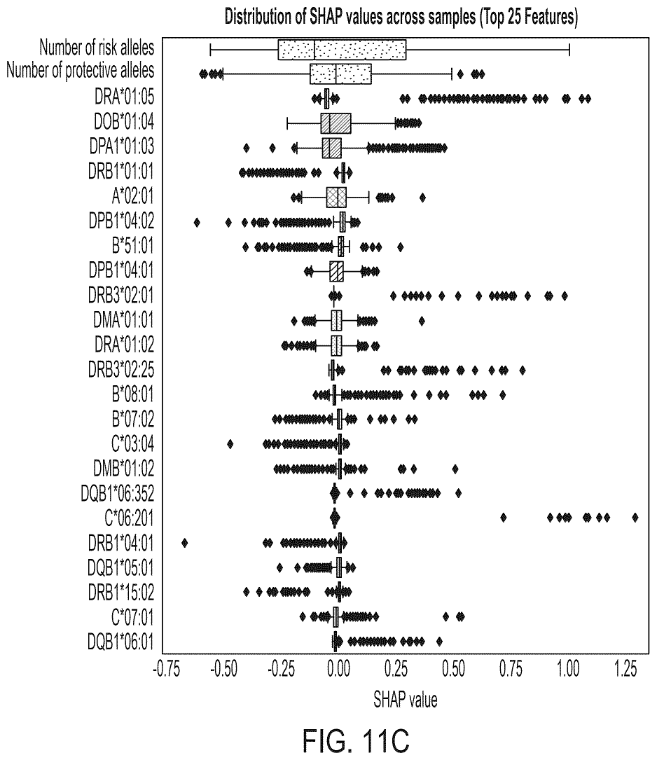

The machine learning model is a gradient-boosted decision tree classifier implemented using CatBoost. The machine learning model was trained on the training autoimmune cohort, with the target variable representing the presence or absence of IBD (Ulcerative colitis or Crohn's disease). Hyperparameter optimization was performed using RandomizedSearchCV from sklearn.model_selection across a predefined parameter space that included the number of boosting iterations (200-1000), learning rate (0.01-0.1), tree depth (4-11), L2 regularization coefficient (2-11), random strength (0.5-1.5), bootstrap type (Bayesian or Bernoulli), and grow policy (Depthwise or SymmetricTree). The search was conducted over 50 iterations using 5-fold cross-validation, optimizing the ROC-AUC score. The final model configuration included 948 boosting iterations, a tree depth of 9, learning rate of 0.022, L2 regularization coefficient of 4, Bayesian bootstrap, class weights set to balanced, and Logloss as the objective function (random seed=42). Feature importances were then derived from the trained classifier and are summarized in Table 30 and illustrated in FIG. 11C. In particular, FIG. 11C shows the distribution of SHAP values across samples for the top 25 most influential features in the HLA feature set.

TABLE 30
HLA alleles and feature importance.

| HLA Allele | Importance | Group |
|---|---|---|
| DPA1*01:03 | 4.232648 | Protective |
| DRA*01:05 | 4.076464 | Risk |
| DMA*01:01 | 3.891708 | Protective |
| A*02:01 | 3.447797 | Risk |
| DPB1*04:01 | 2.526279 | Risk |
| DOB*01:04 | 2.415994 | Risk |
| DRA*01:02 | 2.375175 | Protective |
| DPB1*04:02 | 2.212556 | Protective |
| DRB3*02:25 | 2.16094 | Risk |
| B*51:01 | 2.09355 | Risk |
| B*07:02 | 2.073328 | Protective |
| DRB1*01:01 | 2.056176 | Protective |
| C*07:01 | 1.807696 | Protective |
| DRB3*02:01 | 1.786441 | Risk |
| DMB*01:02 | 1.784069 | Risk |
| C*06:201 | 1.666102 | Risk |
| B*08:01 | 1.398283 | Protective |
| DQB1*05:01 | 1.3632 | Risk |
| C*03:04 | 1.334808 | Protective |
| DRB1*04:01 | 1.326145 | Protective |
| E*01:13 | 1.209694 | Risk |
| DRB1*01:03 | 1.184167 | Risk |
| DQB1*02:01 | 1.124025 | Protective |
| DRB1*15:04 | 1.101392 | Risk |
| C*02:205Q | 1.059023 | Risk |
| DRB1*03:01 | 0.995755 | Protective |
| DRB1*15:02 | 0.978105 | Risk |
| DQB1*06:352 | 0.943292 | Risk |
| DRA*01:07 | 0.919685 | Risk |
| DQB1*06:01 | 0.917363 | Risk |
| DRB1*04:334 | 0.806599 | Risk |
| DRB1*04:07 | 0.778646 | Protective |
| DRB3*01:108 | 0.651119 | Risk |
| DRB1*11:321 | 0.649581 | Risk |
| DQB1*03:518 | 0.633368 | Risk |
| DRB1*01:02 | 0.5887 | Risk |
| DMA*01:06 | 0.43839 | Risk |
| DRB1*07:34 | 0.415876 | Risk |
| DRB3*02:191 | 0.409526 | Risk |
| B*52:01 | 0.264423 | Risk |
| C*12:02 | 0.197327 | Risk |
| DMA*01:05 | 0.173473 | Risk |
| DRA*01:08 | 0.093838 | Risk |
| DQB1*06:395 | 4.232648 | Risk |
| DRB1*13:327 | 4.076464 | Risk |
| DRA*01:06 | 3.891708 | Risk |

### Results

The machine learning model was trained to predict a likelihood that a subject will develop IBD in response to administration of an ICI therapy. The predicted likelihoods were used to stratify patients into: (i) patients likely to develop IBD and (ii) patients not likely to develop IBD. The threshold for stratifying patient was approximately 0.5. For example, a patient for whom the predicted likelihood was greater than or equal to 0.5 was identified as a patient likely to experience a severe irAE.

The machine learning model successfully predicted IBD occurrence in patients with severe irAEs from the RADIOHEAD dataset (AUC=0.68) and in an independent open-source validation cohort (AUC=0.70) (FIGS. 11A and 11B). These results indicate that incorporating HLA genotypes provides complementary predictive value, particularly for irAEs with strong autoimmune components such as IBD.

### B. Example 2

Example 2 demonstrates the performance of techniques used to predict the likelihood that a subject will experience a severe immune-related adverse event (irAE) in response to administration of an ICI therapy.

### 1. Datasets

Multiple datasets were used in this example, including: the PICI Liang Pancancer Radiohead (“RADIOHEAD”) Dataset, MGH Sullivan SKCM IOPROF (“MGH”) Dataset, and a plurality of open-source datasets.

### RADIOHEAD Dataset

Table 31 lists characteristics of patients in the RADIOHEAD dataset.

TABLE 31
Characteristics of patients in the RADIOHEAD dataset.

| Category | All patients, N (%) | |
|---|---|---|
| Patients, (N) | 965 | |
| Age, (median, range) | 69.0 | (25-89) |
| Sex, M/F | 545 (56.5)/420 (43.5) | |
| Therapy: | | |
| Anti-PD-1 | 501 | (51.9) |
| Anti-PD-1 + Chemotherapy | 141 | (14.6) |
| Anti-PDL1 | 140 | (14.5) |
| Anti-CTLA-4 + Anti-PD-1 | 82 | (8.5) |
| Anti-PDL1 + Chemotherapy | 73 | (7.6) |
| Other | 28 | (2.9) |
| Smoker status: | | |
| Ever | 547 | (56.7) |
| Never | 246 | (25.5) |
| Current | 172 | (17.8) |
| Diagnosis: | | |
| Non Small Cell Lung Carcinoma | 351 | (36.4) |
| Melanoma | 119 | (12.3) |
| Renal Cell Carcinoma | 88 | (9.1) |
| Urinary Bladder Neoplasm | 81 | (8.4) |
| Small Cell Lung Carcinoma | 58 | (6.0) |
| Other | 268 | (27.8) |
| Race: | | |
| White | 881 | (91.3) |
| African American | 49 | (5.1) |
| Asian | 19 | (2.0) |
| Other | 12 | (1.2) |
| Hawaii Pacific | 3 | (0.3) |
| Other | 1 | (0.1) |

### MGH Dataset

The MGH dataset includes pre-treatment PBMCs from a melanoma cohort (n=47) treated with either anti-PD-1 (pembrolizumab; n=23) or anti-PD-1 plus anti-CTLA-4 (nivolumab+ipilimumab; n=24) ICI. The severe irAE incidence was 20% (10/47). Table 32 lists characteristics of patients in the MGH dataset.

TABLE 32
Characteristics of patients in the MGH dataset.

| Category | All patients, N (%) | |
|---|---|---|
| Patients, (N) | 51 | |
| Age, (median, range) | 67.0 | (38-89) |
| Sex, M/F | 30 (58.8)/21 (41.2) | |
| Therapy, Anti-PD-1/Anti-CTLA-4 + | 26 (51.0)/25 (49.0) | |
| Anti-PD-1 | | |
| Diagnosis: | | |
| Cutaneous Melanoma | 33 | (64.7) |
| Melanoma | 12 | (23.5) |
| Mucosal Melanoma | 4 | (7.8) |
| Uveal Melanoma | 2 | (3.9) |

### Open-Source Datasets

The open-source datasets include respective open-source datasets for: the HLA predictor, cellular signatures, pathway transcriptomic signatures, and cellular transcriptomic signatures. The datasets are listed in Table 33.

TABLE 33
Open-source datasets.

| Dataset Name | | Dataset Source |
|---|---|---|
| HLA Predictor | | GSE121578, E-MTAB-6739, GSE92472, GSE143507, |
| | | GSE161031, GSE159034, GSE171770, GSE191328, |
| | | GSE177044, GSE186507, GSE117875, GSE69446, |
| | | GSE95450, GSE99816, E-MTAB-5464, GSE184307, |
| | | GSE156044, GSE115390, GSE158952, GSE171244, |
| | | GSE199906, GSE224758, GSE192819, GSE57945, |
| | | GSE93624, GSE81266, GSE233900, GSE261086, |
| | | GSE243625, GSE230113, GSE157020, GSE174159, |
| | | GSE137344, GSE83687, GSE228122, GSE164871, |
| | | GSE66207, GSE134080, GSE97356, GSE164877, |
| | | GSE193141, GSE198449, GSE54308, PRJNA938007, |
| | | GSE215067, E-MTAB-9708, E-MTAB-10395, |
| | | GSE192786, GSE151686, GSE215144, GSE139179, |
| | | GSE235236, GSE123141, GSE172372, GSE112057 |
| Cellular | Cytometry | PMID35027754, PMID3166003, PMID34360781, |
| Signatures | | PMID36248910, PMID35074903 |
| | RNA-Seq | GSE186143, GSE180045, GSE216329 |
| Pathway Transcriptomic Signatures | | GSE186143 |
| Cellular | CD4 | GSE103844, GSE114065, GSE129829, GSE75011, |
| Transcriptomic | | GSE133822, GSE94396, GSE80016, GSE113891, |
| Signatures | | GSE121827, GSE90569, GSE117655, GSE87505, |
| | | GSE96538, GSE130882, GSE52260, GSE122612, |
| | | GSE104744, GSE114407, GSE94150, GSE95297, |
| | | GSE94149, GSE116073, GSE161829, GSE94859, |
| | | GSE73213, GSE78276, GSE116139, GSE143213, |
| | | GSE65621, GSE102045, GSE114883, GSE89225, |
| | | GSE110417, GSE60424, GSE84445, GSE172317, |
| | | GSE134416, GSE56179, GSE123812, GSE66763, |
| | | GSE112101, E-MTAB-6370, GSE86452, GSE94964, |
| | | E-MTAB-2319, GSE78922, GSE97862, GSE89404, |
| | | GSE60482, GSE150805, GSE71645, GSE122735, |
| | | GSE124757, GSE111377, GSE127457, GSE129522, |
| | | GSE107011, GSE83808, GSE59846, GSE130580, |
| | | GSE95754, GSE130810, GSE118974, GSE115898, |
| | | GSE118951, GSE122321, GSE97861, GSE110097, |
| | | GSE139341, GSE87399, GSE114716, GSE107981, E- |
| | | MTAB-5622, GSE150834, GSE85294, GSE84197, |
| | | PRJNA486998, GSE115103, GSE125504, GSE90468, |
| | | GSE149219, GSE148669, GSE118094 |
| | T cell | GSE121827, GSE103844, GSE133822, GSE114065, |
| | | GSE111892, GSE90730, GSE87505, GSE129829, |
| | | GSE104744, GSE120904, GSE75011, GSE94396, |
| | | GSE114407, GSE80016, GSE113891, GSE134416, |
| | | GSE90569, GSE117655, GSE96538, GSE107011, |
| | | GSE130882, GSE52260, GSE131088, GSE131089, |
| | | GSE122612, GSE87517, GSE84445, GSE60424, |
| | | GSE99531, GSE126752, GSE94150, GSE95297, |
| | | GSE83637, GSE94149, GSE116073, GSE113590, E- |
| | | MTAB-6370, GSE158835, GSE94859, GSE122624, |
| | | GSE161829, GSE123977, GSE123649, GSE73213, |
| | | GSE94964, E-MTAB-2319, GSE116139, GSE78276, |
| | | GSE143213, GSE65621, GSE112483, GSE97862, |
| | | GSE83808, GSE114883, GSE102045, E-MTAB-7143, |
| | | GSE89225, GSE129196, GSE110417, GSE122149, |
| | | GSE111377, GSE172317, GSE139341, GSE111389, |
| | | GSE141797, GSE115898, GSE63144, GSE113098, |
| | | GSE56179, GSE66763, GSE135582, GSE110469, |
| | | GSE123812, GSE140430, GSE112101, GSE95754, E- |
| | | MTAB-5381, GSE100624, GSE130810, GSE119918, |
| | | GSE86452, GSE100860, GSE89134, GSE78922, |
| | | GSE110097, GSE89404, GSE129522, GSE141645, |
| | | GSE106420, GSE129906, GSE122735, GSE115305, |
| | | GSE127457, E-MTAB-5640, GSE124757, GSE60482, |
| | | GSE150805, GSE80306, GSE109841, GSE71645, |
| | | GSE130580, GSE59846, GSE78522, GSE90600, |
| | | GSE132812, GSE128822, GSE117614, GSE164266, |
| | | GSE76371, GSE58596, GSE115736, GSE118974, |
| | | GSE87399, GSE97861, GSE85294, E-MTAB-6727, |
| | | GSE115686, GSE124876, GSE122321, GSE125504, |
| | | GSE123805, GSE118951, GSE147620, GSE81975, |
| | | GSE124381, GSE107981, GSE150834, GSE112341, |
| | | GSE135291, GSE116015, GSE111968, GSE120847, |
| | | GSE64655, GSE140483, E-MTAB-5622, GSE114716, |
| | | GSE74246, GSE116865, GSE84197, GSE84531, |
| | | GSE117627, GSE149219, GSE106830, GSE148669, |
| | | GSE135390, GSE110684, GSE118094, GSE69239, |
| | | GSE144108, GSE120364, GSE162179, GSE96578, |
| | | GSE115103, GSE90468, PRJNA486998, GSE155715 |
| | PBMC | GSE120596, GSE103401, GSE96783, GSE102288, |
| | | GSE120502, GSE152683, GSE150735, GSE166292, |
| | | GSE169030, GSE168698, GSE168409, GSE164366, |
| | | GSE135192, GSE162562, GSE79970, GSE131590, |
| | | GSE163527, GSE184039, GSE115449, GSE109515, |
| | | GSE119117, GSE135964, GSE138746, GSE120115, |
| | | GSE58122, GSE122058, GSE182522, GSE179627, |
| | | GSE122438, GSE165149, GSE162746, GSE110146, |
| | | GSE113210, GSE114588, GSE85263, GSE133298, E- |
| | | MTAB-6270, GSE81259, GSE112104, GSE141646, |
| | | GSE102677, GSE154911, GSE114407, GSE94892, |
| | | GSE157859, GSE111405, E-MTAB-8249, |
| | | GSE165604, GSE110325, GSE174566, GSE166761, |
| | | GSE152418, GSE151159, GSE182038, GSE128627, |
| | | GSE158712, GSE134985, GSE108665, GSE161031, |
| | | GSE156124, GSE142514, GSE163605, GSE104423, E- |
| | | MTAB-9066, GSE174072, GSE113287, GSE166253, |
| | | GSE165254, GSE32874, GSE125223, GSE164208, |
| | | GSE79027, GSE138804, GSE58335, GSE179621, |
| | | GSE161199, GSE156336, GSE134979, GSE175988, |
| | | GSE77929, GSE98884, GSE126091, GSE153122, |
| | | GSE35394, GSE159094, GSE129534, GSE123786, |
| | | GSE179987, GSE119835, GSE74235, GSE122709, |
| | | GSE100026, GSE120663, GSE153100, GSE92917, |
| | | GSE163073, GSE159337, GSE133499, GSE152179, |
| | | GSE154703, GSE122309, GSE183817, GSE107011, |
| | | GSE94800, GSE123523, GSE60217, GSE115259 |
| | CD8 | GSE111892, GSE90730, GSE120904, GSE133822, |
| | | GSE87505, GSE104744, GSE114407, GSE99531, |
| | | GSE83637, GSE113590, GSE122149, GSE80306, |
| | | GSE84445, GSE111389, GSE113098, GSE63144, |
| | | GSE60424, GSE140430, E-MTAB-6370, GSE135582, |
| | | GSE107011, GSE78522, GSE100624, E-MTAB-5381, |
| | | GSE94964, GSE119918, GSE100860, GSE89134, E- |
| | | MTAB-5640, GSE109841, GSE115305, GSE106420, |
| | | GSE83808, E-MTAB-2319, GSE132812, GSE117614, |
| | | E-MTAB-6727, GSE111377, GSE147620, |
| | | GSE141645, GSE135291, GSE116865, GSE115898, |
| | | GSE81975, GSE96578, GSE155715, GSE144108, |
| | | GSE162179, GSE110684 |

### 2. Predictors

Multiple predictors were developed to predict whether a subject will experience an immune-related adverse event in response to administration of an ICI therapy including and/or to predict whether a subject will develop a specific immune-related adverse event in response to administration of an ICI. The predictors include: an HLA predictor, a cellular signature predictor, a pathway transcriptomic signatures predictor, a cellular transcriptomic signatures predictor, an immunotypes predictor, and an embeddings predictor.

### HLA Predictor

The HLA predictor model integrates data from both internal data and literature sources to identify alleles associated with autoimmune diseases. This model is built upon an analysis of unique alleles present in both diseased and healthy cohorts, as well as significant alleles reported in the literature.

For the diseased cohort, RNA-Seq open-source autoimmune datasets were used to identify unique patients based on SNP (STAR) data. This cohort included patients with diseases listed in Table 34 (for example, a total of 1884 patients with Diarrhea Colitis). For the healthy cohort, RNA-Seq open-source healthy datasets and laboratory data were used, comprising a total of 880 healthy patients.

Subsequently, for each locus, sub-cohorts were formed within both the diseased and healthy cohorts, depending on locus typing. Attention was given to maintaining the ethnicity ratio in the diseased cohort so that the healthy cohort reflects a matching ethnicity distribution.

After identifying unique alleles, statistical analysis was performed. The odds ratio and p-value were calculated using the Barnard test, followed by adjustment of the p-values using the FDR correction method (Benjamini-Hochberg). Based on this analysis, a list of unique disease risk or protective alleles for all loci was compiled.

Simultaneously, a literature review was conducted on autoimmune diseases. Articles were annotated in the HLA Database (HLA_v.2.0) to identify significant alleles and other data. The database of Anthony Nolan Research Institute (hla.alleles.org/alleles/deleted.html was used to check for an actual list of alleles. For each diagnosis, literature-derived alleles were filtered to retain only those appearing more than once and exhibiting consistent effects, meaning they influence the odds ratio in the same direction.

The collected literature data was used to create a list of unique disease risk and protective alleles for all loci. Then it was combined with previously identified alleles to enhance the statistical power of the analysis.

To enhance the models' generalization, reduce variance, and improve robustness against outliers additional healthy cohort (1329 patients) was used, increasing the training dataset size for validation. The presence of each allele was confirmed within the samples, along with information about the typing for each locus.

Based on this information, the presence of unique alleles in the cohorts was analyzed, and a predictive model was trained using the CatBoost algorithm. This model aims to confirm the presence of autoimmune diseases based on the identified alleles.

The following alleles were selected as features for the model.

Identified alleles: B*35:01, C*03:04, DMA*01:01, DMA*01:03, DMA*01:04, DMA*01:06, DMB*01:01, DMB*01:02, DMB*01:04, DOB*01:01, DOB*01:04, DPA1*01:03, DPB1*03:01, DQB1*02:01, DQB1*06:09, DRA*01:02, DRA*01:05, DRB1*01:01, DRB1*04:01, DRB3*01:108, DRB3*02:01, DRB3*02:02, DRB3*02:25, DRB5*01:01, DRB5*01:08, DRB5*01:119, DRB5*01:53N.

Literature alleles: A*02:01, B*07:02, B*08:01, B*51:01, B*52:01, C*07:01, C*12:02, DPB1*04:01, DQB1*05:01, DQB1*06:01, DRB1*01:03, DRB1*03:01, DRB1*15:02, DQB1*02:01, DRB1*04:01.

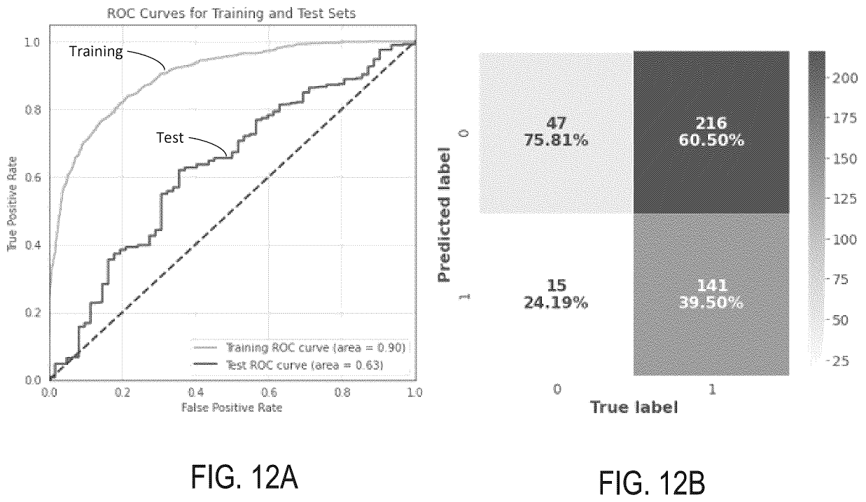

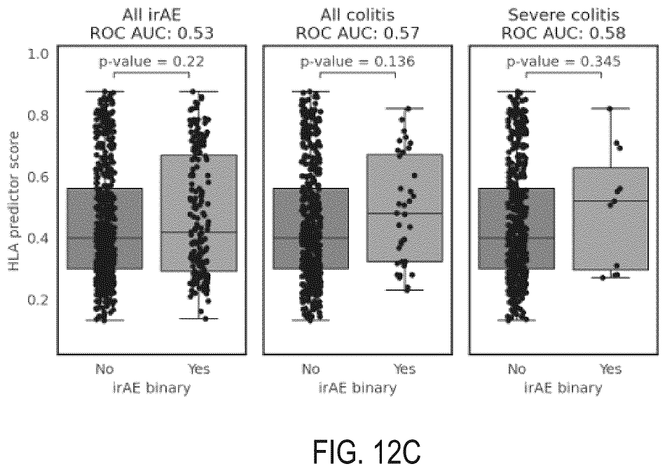

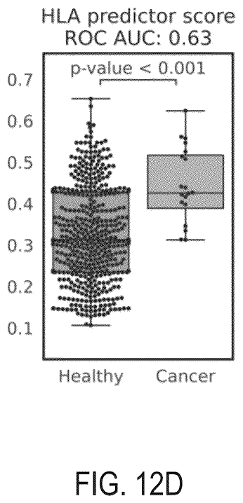

FIGS. 12A-12C present the results of model evaluation on the test dataset and real-world data. FIG. 12A is a plot that shows the ROC-AUC curve for the test dataset, illustrating the model's accuracy in distinguishing between classes. In FIG. 12B, the confusion matrix displays the distribution of correct and incorrect predictions on the test set. FIG. 12C contains boxplots for real-world data, visualizing the distribution of predicted values within each group and enabling an assessment of differences between them.

As part of the validation methods, the HLA score in colorectal cancer patients was evaluated. FIG. 12D presents a boxplot demonstrating a significant separation between two patient groups based on their HLA scores. This result is consistent with expectations, given the strong association between colorectal cancer and colitis.

TABLE 34
Immune-related adverse events and related diagnoses.

| irAE name | Related diagnoses from datasets |
|---|---|
| Diarrhea Colitis | Crohn's disease, Ulcerative colitis, Inflammatory bowel disease |
| Pneumonitis | Idiopathic pulmonary fibrosis, Systemic sclerosis-associated |
| | interstitial lung disease, Interstitial lung disease, Rheumatoid |
| | arthritis-associated interstitial lung disease, Non-usual |
| | interstitial pneumonia, Interstitial lung disease in Primary |
| | Sjogren syndrome, Idiopathic interstitial pneumonia |
| Hepatitis | Autoimmune hepatitis, ICI hepatitis |
| Myocarditis | Myocarditis, ICI myocarditis, Vaccine-associated myocarditis, |
| | Cardiac sarcoidosis |
| Cytokine release | Systemic inflammatory response syndrome, Sepsis, COVID-19, |
| syndrome | Septic shock, Multisystem Inflammatory Syndrome in Children |
| Systemic inflammatory | Systemic inflammatory response syndrome, Sepsis, COVID-19, |
| response syndrome | Multisystem Inflammatory Syndrome in Children |
| Diabetes mellitus | Type 1 diabetes mellitus |
| Arthritis | Rheumatoid arthritis, Osteoarthritis, ICI arthritis, Psoriatic |
| | arthritis |
| Myositis | Polymyositis, Dermatopolymyositis, Inclusion Body Myositis |
| Myasthenia gravis | Myasthenia gravis |
| Guillain-Barre syndrome | Guillain-Barre syndrome |
| Nephritis | Nephritis, Glomerulonephritis, GN-membranous |
| | glomerulonephritis, Membranoproliferative glomerulonephritis |
| Hypothyroidism | Hypothyroidism |

### Cellular Signature

Pre-treatment PBMCs from patients with advanced melanoma were profiled using flow cytometry and bulk RNA sequencing. A cellular irAE signature was developed using 17 peripheral immune cell populations selected from open-source data. Principal component analysis was then used to evaluate these populations in samples from the melanoma cohort. Additionally, a gene-based irAE signature was developed from 55 reported irAE-associated genes using ssGSEA for gene signature calculation.

The development of a cell prediction model involved three stages. The stages helped to ensure the accuracy and reliability of the model.

In the initial stage, populations from publicly available cytometry and RNA sequencing datasets were selected that are identical to BostonGene's internal cell populations. For cytometry data, available calculated percentages of cell types from open-source databases were used.

To identify cell populations from bulk RNA sequencing data, the Kassandra deconvolution model was applied (science.bostongene.com/kassandra/), which allowed for accurate estimation of the proportions of various cell types from the RNA sequencing data.

After this, all cell population percentages were normalized to the parent population percentage to ensure consistency across datasets and reduce the impact of variations in larger populations on smaller ones.

Following the normalization process, a differential analysis was performed to identify cell populations with statistically significant differences between patients with severe irAE and patients without. Populations that were significantly different between patients with irAEs and those without severe irAEs (MWU test, a p-value<0.05) were identified. As a result, 17 cell populations that were associated with irAE occurrence were identified.

In the second stage, there was a focus on defining connections between the identified cellular populations using principal component analysis (PCA). For training the PCA model, an internal cohort consisting of approximately 1,000 patients (doi.org/10.1016/j.ccell.2024.04.008) was utilized. The second principal component (PCA2) was selected as the signature differentiating patients with serious adverse events following ICI therapy.

The final step was to validate the developed cellular predictor using an independent melanoma cohort (including 47 patients, 10 with severe irAEs). This validation demonstrated the model's potential to effectively predict severe adverse events in this clinical setting. The development of the Cellular irAE Signature was carried out in three stages: identification of cell populations in open datasets, integration of these cell populations using principal component analysis (PCA) and an independent cohort to calculate the signature for the melanoma cohort, and analysis of the signature along with assessment of feature importance, particularly focusing on principal component 2 (PC2).

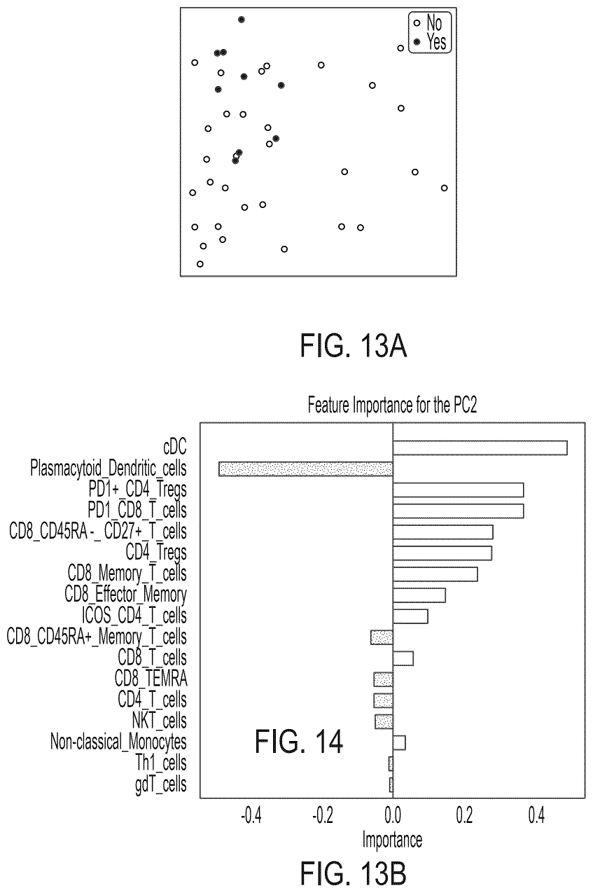

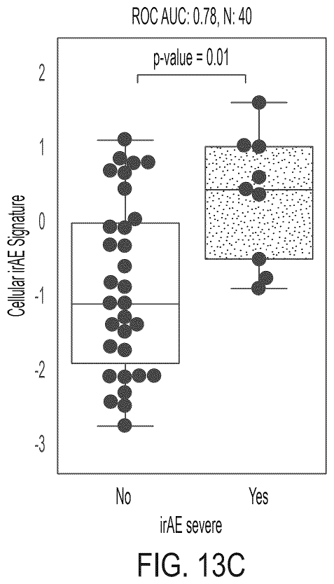

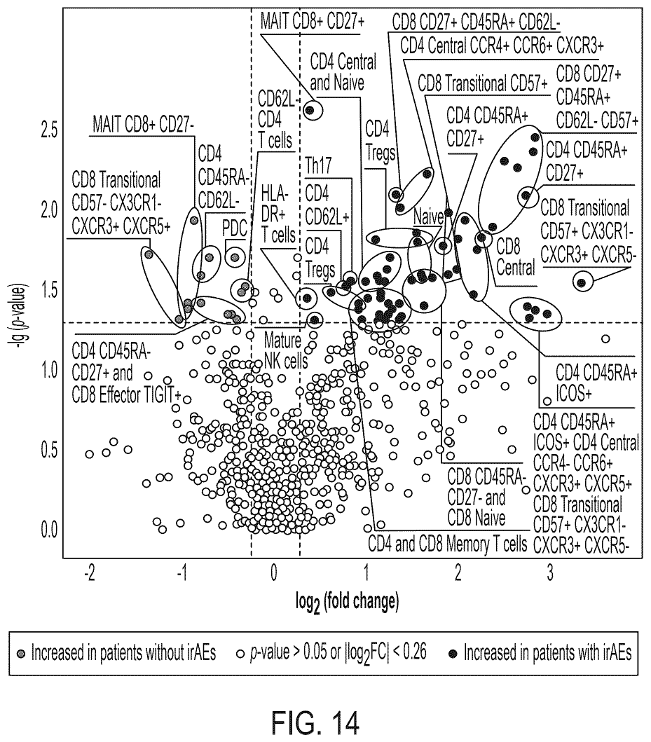

Patients with severe irAEs had significantly different cellular signature (PC2) at baseline from those without irAEs (ROC-AUC=0.78, p=0.01; FIGS. 12A-12C), FIGS. 12A-12C show the performance of the cellular irAE signature in the MGH cohort (irAE: yes=9, no=31). FIG. 13A shows the cellular PC2 for patients with severe irAEs. FIG. 13B shows the signature features (PC2) sorted by weights. FIG. 13C shows the distribution of patients with and without severe irAEssein the cellular principal component space. An independent differential cell population analysis of the MGH cohort identified cellular clusters that distinguished between patients with and without irAEs (FIG. 14).

Table 35 lists the weights of the PCA.

TABLE 35
Weights of the PCA.

| Population | Weight |
|---|---|
| CDC | 0.4856077908340596 |
| Plasmacytoid Dendritic cells | −0.4856077908340579 |
| CD4 Tregs PD-1+ | 0.3648508734571471 |
| CD8 T cells PD-1+ | 0.36378953162177363 |
| CD8 CD45RA- CD27+ T cells | 0.2799396362504675 |
| CD4 Tregs | 0.2785682236742606 |
| CD8 Memory T cells | 0.23815544468122993 |
| CD8 Effector Memory | 0.1497952875576699 |
| CD4 T cells ICOS+ | 0.09964264641629443 |
| CD8 CD45RA+ Memory T cells | −0.0644494515192163 |
| CDS T cells | 0.058758650311803086 |
| CD8 TEMRA | −0.05487328401111803 |
| CD4 T cells | −0.0546502530369101 |
| NKT cells | −0.05123658121017785 |
| Non-classical Monocytes | 0.03683100096466562 |
| Th1 cells | −0.013237565810203574 |
| gdT cells | −0.009921004448920263 |

### Pathway Transcriptomic Signatures Predictor

The development of the Gene irAE Signature involved identifying irAE-associated genes, filtering for correlation, and assembling the differentiating genes into a signature using ssGSEA.

A comprehensive search was conducted for biomarkers that can differentiate patients with or without irAE (or with grade 0-2 vs grade 3-4 irAE) and can be evaluated based on the transcriptome data, for the development of irAE predictor. The biomarkers that were checked included:
1. protein or gene expression biomarkers obtained from a detailed review of the literature, with over 40 publications processed;
2. Differential expression analysis;
3. Functional gene expression signatures (FGES) developed previously in the BostonGene company (Bagaev et al., 2021) and newly composed, and single genes consist of;
4. Cell fractions calculated by the Kassandra deconvolution tool (DOI: doi.org/10.1016/j.ccell.2022.07.006).

All selected biomarkers were tested on the internal and publicly available datasets treated with immune checkpoint inhibitors (ICI) and annotated for irAE, which are listed in Table 36, for their ability to differentiate patients with irAE, and especially high-grade irAE, at baseline or already on treatment.

TABLE 36
Data used in the evaluation of biomarker performance.

| | | | N | | | |
|---|---|---|---|---|---|---|
| | | | without | N with | N irAE | N irAE |
| Dataset ID | Data type | Diagnosis | irAE | irAE | grade 0-2 | grade 3-4 |
| Internal 1 | Bulk | Melanoma | 37 | 10 | n/a | n/a |
| (baseline) | RNAseq | | | | | |
| Internal 1 | Bulk | Melanoma | 37 | 8 | n/a | n/a |
| (day 21) | RNAseq | | | | | |
| Internal 1 | Bulk | Melanoma | 33 | 6 | n/a | n/a |
| (day 42) | RNAseq | | | | | |
| Internal 1 | Bulk | Melanoma | 30 | 9 | n/a | n/a |
| (day 63) | RNAseq | | | | | |
| Internal 2 | Bulk | HNSCC | 23 | 8 | n/a | n/a |
| | RNAseq | | | | | |
| GSE186143 | Bulk | Melanoma | 10 | 37 | 30 | 17 |
| | RNAseq | | | | | |
| GSE180045 | Single-cell | NSCLC, | 2 | 7 | n/a | n/a |
| | RNAseq | HNSCC, | | | | |
| | (turned into | SKCM, | | | | |
| | pseudo-bulk) | BLCA, | | | | |
| | | Adrenal | | | | |
| | | Cancer | | | | |

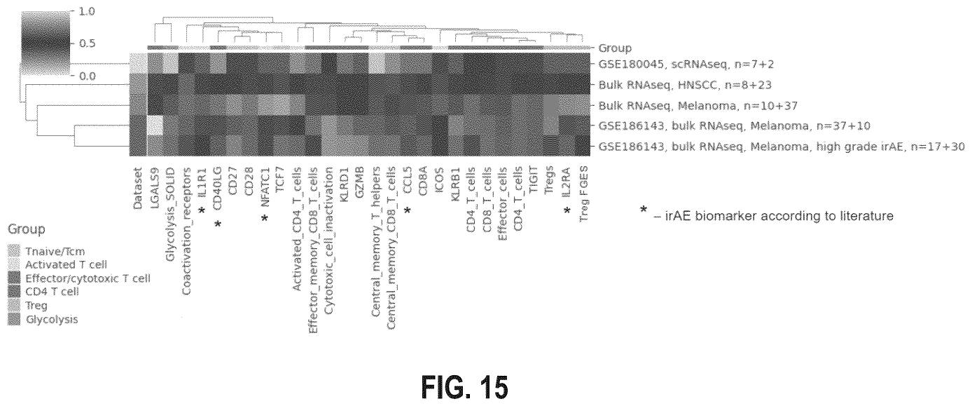

FIG. 15, Table 37, and Table 38 show the ROC-AUC values and the corresponding p-values of the baseline biomarkers, which have demonstrated ROC-AUC>0.5 in two or more independent datasets and, thus, have the potential for the prediction of irAE development risk. These biomarkers cluster into certain biological groups. One group is formed by regulatory T cells, defined both by Treg FGES and Treg deconvolution model, and a single gene IL2RA, which is also commonly and rather specifically expressed on regulatory T cells. Interestingly, a higher signal from Tregs is significantly associated with the risk of irAE in two datasets, in controversy with previously published data where the lower amount was shown to be associated with toxicity development [PMID: 28368458]. The next two clusters of features unite cytotoxic and CD4 T cells, different types of memory T cells, and several genes of inhibitory receptors (KLRD1, KLRB1, TIGIT), cytotoxic cell markers (GZMB, CCL5, CD8A) and activation marker ICOS. Higher levels of main T cell subpopulations like CD4+ or CD8+ T cells are proven to be connected to irAE development already [PMID: 35892826, 33980577, 35027754]. Another cluster unites markers of activated T cells and naive/central memory T cells (ILIR1, CD40LG, CD27, CD28, NFATC1, TCF7). Activated subsets of memory T cells are associated with severe irAE in several publications (PMID 37794264, 37035636, 35027754). The last cluster includes glycolysis FGES and LGALS9 gene. The level of LDH, one of the important glycolytic enzymes, was shown to have associations with irAE development (PMID: 35192899). Galectin-9 was reported as a predictor of adverse events only for patients with chronic HIV during suppressive antiretroviral therapy (PMID: 34366381).

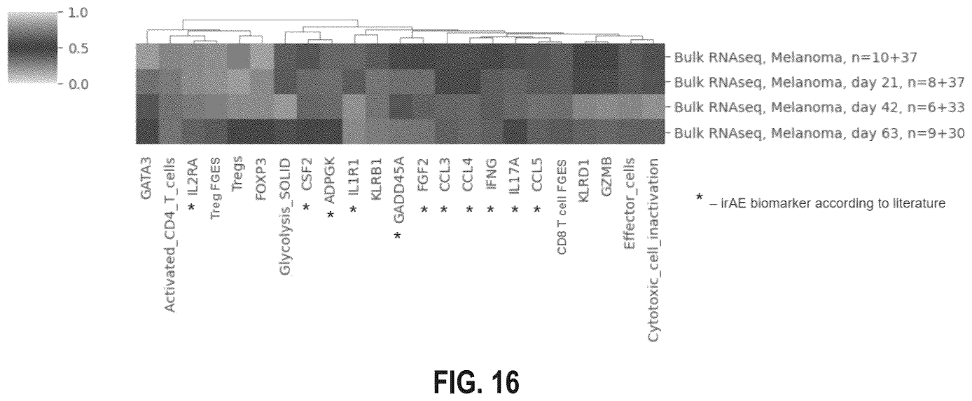

The same analysis was conducted for the dynamic biomarkers, and the corresponding genes, FGES, and deconvolution scores were tested on the dataset of melanoma patients screened at several time points (Internal dataset 1, see Table 36). FIG. 16, Table 39, and Table 40 show ROC-AUC values and the corresponding p-values of the dynamic biomarkers, which have demonstrated ROC-AUC>0.5 in two or more time points and, thus, have the potential for prediction of irAE development risk during immunotherapy treatment.

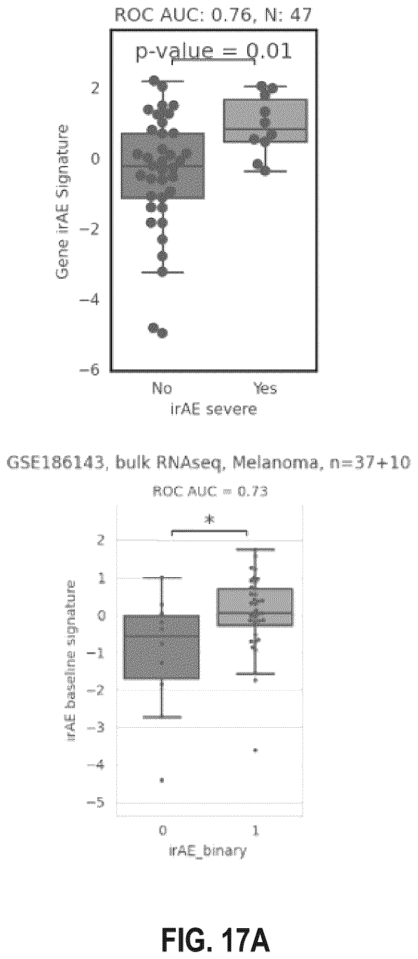

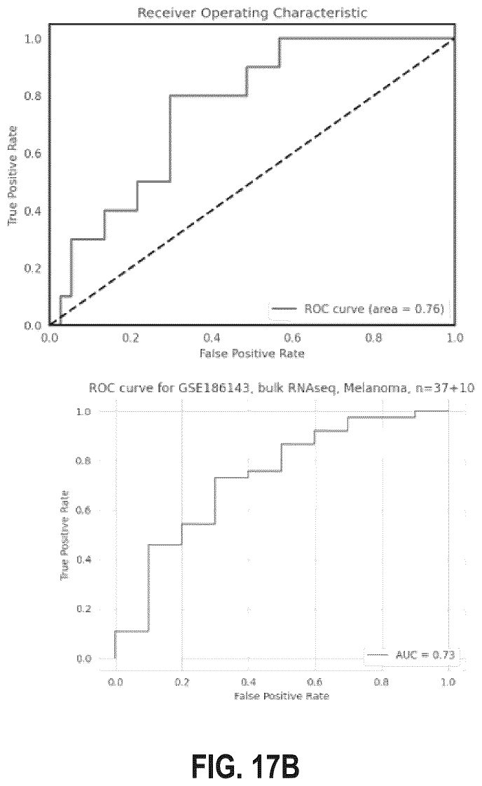

Based on the selected biomarkers, which have passed the test of their ability to differentiate samples with and without irAE, a unified score calculated on transcriptomic data is developed to predict which patient is going to develop irAE during immunotherapy treatment. According to the results presented above, this score may be based on genes related to certain blood cell populations, cytokines, markers of cell activation, cytotoxicity, and/or cell metabolism. One variant of the risk score may be based on baseline markers, i.e., defined before the beginning of the therapy regimen. The other variant may be based on biomarkers that have shown prediction ability in samples already on treatment, i.e., dynamic biomarkers. The preliminary results of baseline predictor performance are seen in FIGS. 17A-17B, where the irAE biomarker score differentiates patients with irAE in two training melanoma datasets (internal 1 and GSE186143) with an average ROC-AUC value of 0.745. FIG. 17A shows the gene signature calculated by ssGSEA for patients with severe irAEs (MGH.Sullivan.SKCM.IOPROF and GSE186143). FIG. 17B shows the ROC-AUC curve of the irAE gene signature score (MGH.Sullivan.SKCM.IOPROF and GSE186143).Such predictors will allow to minimize risks of severe complications if immunotherapy is regarded as the best choice for an individual patient.

TABLE 37
ROC-AUC values of baseline biomarkers from different sources were tested for the ability
to differentiate patients with irAE vs patients without irAE (or patients with grade
3-4 irAE vs grade 0-2 irAE where indicated) in each test dataset and averaged.

| | | | GSE186143 | | GSE180045 | |
|---|---|---|---|---|---|---|
| | Internal 1 | GSE186143 | high-grade | Internal 2 | (mixed | |
| | (Melanoma) | (Melanoma) | (Melanoma) | (HNSCC) | diagnoses) | Average |
| LGALS9 | 0.54 | 0.99 | 0.78 | 0.57 | 0.79 | 0.734 |
| Central_memory_T_helpers | 0.61 | 0.65 | 0.69 | 0.41 | 0.93 | 0.658 |
| (deconv) | | | | | | |
| Cytotoxic_cell_inactivation | 0.58 | 0.81 | 0.81 | 0.56 | 0.5 | 0.652 |
| (FGES) | | | | | | |
| KLRB1 | 0.61 | 0.75 | 0.69 | 0.54 | 0.64 | 0.646 |
| KLRD1 | 0.5 | 0.72 | 0.79 | 0.48 | 0.71 | 0.64 |
| Glycolysis_SOLID (FGES) | 0.38 | 0.72 | 0.69 | 0.45 | 0.93 | 0.634 |
| Tregs (deconv) | 0.74 | 0.75 | 0.59 | 0.45 | 0.64 | 0.634 |
| IL2RA | 0.77 | 0.6 | 0.66 | 0.49 | 0.64 | 0.632 |
| CD4_T_cells (deconv) | 0.68 | 0.68 | 0.68 | 0.49 | 0.57 | 0.62 |
| CD27 | 0.79 | 0.64 | 0.75 | 0.47 | 0.43 | 0.616 |
| TCF7 | 0.85 | 0.58 | 0.7 | 0.59 | 0.36 | 0.616 |
| Central_memory_CD8_T_cells | 0.57 | 0.61 | 0.7 | 0.39 | 0.79 | 0.612 |
| (deconv) | | | | | | |
| GZMB | 0.49 | 0.65 | 0.77 | 0.51 | 0.64 | 0.612 |
| Tregs (FGES) | 0.79 | 0.62 | 0.51 | 0.48 | 0.64 | 0.608 |
| CD8A | 0.61 | 0.59 | 0.66 | 0.47 | 0.71 | 0.608 |
| TIGIT | 0.66 | 0.66 | 0.65 | 0.47 | 0.57 | 0.602 |
| CCL5 | 0.6 | 0.64 | 0.61 | 0.43 | 0.71 | 0.598 |
| Effector_memory_CD8_T_cells | 0.59 | 0.69 | 0.44 | 0.56 | 0.71 | 0.598 |
| (deconv) | | | | | | |
| CD8_T_cells (FGES) | 0.62 | 0.68 | 0.69 | 0.46 | 0.5 | 0.59 |
| CD4_T_cells (FGES) | 0.68 | 0.69 | 0.58 | 0.49 | 0.5 | 0.588 |
| CD28 | 0.72 | 0.66 | 0.67 | 0.43 | 0.43 | 0.582 |
| Effector_cells (FGES) | 0.61 | 0.64 | 0.65 | 0.43 | 0.57 | 0.58 |
| Activated_CD4_T_cells | 0.76 | 0.5 | 0.33 | 0.51 | 0.79 | 0.578 |
| (FGES) | | | | | | |
| NFATC1 | 0.82 | 0.56 | 0.57 | 0.45 | 0.36 | 0.552 |
| ICOS | 0.66 | 0.49 | 0.53 | 0.4 | 0.57 | 0.53 |
| CD40LG | 0.71 | 0.58 | 0.64 | 0.5 | 0.21 | 0.528 |
| Coactivation_receptors | 0.69 | 0.65 | 0.4 | 0.34 | 0.43 | 0.502 |
| (FGES) | | | | | | |
| IL1R1 | 0.66 | 0.42 | 0.5 | 0.55 | 0.36 | 0.498 |

Deconv = deconvolution by Kassandra algorithm.
FGES = functional gene expression signatures, calculated by ssGSEA.

TABLE 38
P-values of ROC-AUC of baseline biomarkers from different sources tested for the
ability to differentiate patients with irAE vs patients without irAE (or patients
with grade 3-4 irAE vs grade 0-2 irAE where indicated) in each test dataset.

| | | | GSE186143 | | GSE180045 |
|---|---|---|---|---|---|
| | Internal 1 | GSE186143 | high-grade | Internal 2 | (mixed |
| | (Melanoma) | (Melanoma) | (Melanoma) | (HNSCC) | diagnoses) |
| LGALS9 | 0.706 | 0.001 | 0.014 | 0.580 | 0.333 |
| Central_memory_T_{—} | 0.317 | 0.164 | 0.036 | 0.464 | 0.111 |
| helpers (deconv) | | | | | |
| Cytotoxic_cell_{—} | 0.443 | 0.003 | 0.001 | 0.642 | 1.000 |
| inactivation (FGES) | | | | | |
| KLRB1 | 0.317 | 0.016 | 0.031 | 0.774 | 0.667 |
| KLRD1 | 0.990 | 0.122 | 0.004 | 0.912 | 0.500 |
| Glycolysis_SOLID | 0.258 | 0.034 | 0.033 | 0.707 | 0.111 |
| (FGES) | | | | | |
| Tregs (deconv) | 0.022 | 0.017 | 0.335 | 0.707 | 0.667 |
| IL2RA | 0.009 | 0.343 | 0.071 | 0.947 | 0.667 |
| CD4_T_cells (deconv) | 0.089 | 0.079 | 0.043 | 0.982 | 0.889 |
| CD27 | 0.006 | 0.189 | 0.004 | 0.842 | 0.889 |
| TCF7 | 0.001 | 0.475 | 0.025 | 0.464 | 0.667 |
| Central_memory_{—} | 0.507 | 0.292 | 0.022 | 0.391 | 0.333 |
| CD8_T_cells (deconv) | | | | | |
| GZMB | 0.907 | 0.142 | 0.003 | 0.947 | 0.667 |
| Tregs (FGES) | 0.005 | 0.269 | 0.921 | 0.912 | 0.667 |
| CD8A | 0.281 | 0.370 | 0.078 | 0.808 | 0.500 |
| TIGIT | 0.135 | 0.116 | 0.104 | 0.842 | 0.889 |
| CCL5 | 0.330 | 0.189 | 0.236 | 0.580 | 0.500 |
| Effector_memory_{—} | 0.397 | 0.075 | 0.472 | 0.635 | 0.500 |
| CD8_T_cells (deconv) | | | | | |
| CD8_T_cells (FGES) | 0.269 | 0.094 | 0.036 | 0.740 | 1.000 |
| CD4_T_cells (FGES) | 0.084 | 0.071 | 0.347 | 0.947 | 1.000 |
| CD28 | 0.036 | 0.116 | 0.058 | 0.611 | 0.889 |
| Effector_cells (FGES) | 0.281 | 0.181 | 0.082 | 0.611 | 0.889 |
| Activated_CD4_T_{—} | 0.011 | 0.990 | 0.061 | 0.982 | 0.333 |
| cells (FGES) | | | | | |
| NFATC1 | 0.002 | 0.576 | 0.458 | 0.674 | 0.667 |
| ICOS | 0.135 | 0.907 | 0.782 | 0.411 | 0.889 |
| CD40LG | 0.050 | 0.459 | 0.124 | 1.000 | 0.333 |
| Coactivation_{—} | 0.067 | 0.164 | 0.273 | 0.203 | 0.889 |
| receptors (FGES) | | | | | |
| IL1R1 | 0.116 | 0.532 | 0.982 | 0.707 | 0.667 |

TABLE 39
ROC-AUC values of dynamic biomarkers from different sources were
tested for the ability to differentiate patients with irAE vs patients
without irAE at each time point indicated and averaged.

| | | Internal 1 | Internal 1 | Internal 1 | |
|---|---|---|---|---|---|
| | Internal 1 | (Melanoma), | (Melanoma), | (Melanoma), | |
| | (Melanoma) | day 21 | day 42 | day 63 | Average |
| Tregs (FGES) | 0.79 | 0.8 | 0.74 | 0.61 | 0.735 |
| IL2RA | 0.77 | 0.78 | 0.72 | 0.64 | 0.7275 |
| FOXP3 | 0.84 | 0.77 | 0.78 | 0.49 | 0.72 |
| Activated_CD4_T_cells | 0.76 | 0.72 | 0.69 | 0.7 | 0.7175 |
| (FGES) | | | | | |
| IL1R1 | 0.66 | 0.62 | 0.79 | 0.77 | 0.71 |
| Tregs | 0.74 | 0.83 | 0.78 | 0.48 | 0.7075 |
| KLRB1 | 0.61 | 0.7 | 0.7 | 0.73 | 0.685 |
| GATA3 | 0.83 | 0.69 | 0.6 | 0.54 | 0.665 |
| Effector_cells (FGES) | 0.61 | 0.61 | 0.76 | 0.64 | 0.655 |
| Cytotoxic_cell_inactivation | 0.58 | 0.59 | 0.8 | 0.64 | 0.6525 |
| (FGES) | | | | | |
| CD8_T_cells (FGES) | 0.62 | 0.62 | 0.68 | 0.61 | 0.6325 |
| CCL5 | 0.6 | 0.61 | 0.69 | 0.63 | 0.6325 |
| GADD45A | 0.55 | 0.68 | 0.59 | 0.71 | 0.6325 |
| GZMB | 0.49 | 0.56 | 0.78 | 0.66 | 0.6225 |
| FGF2 | 0.47 | 0.68 | 0.63 | 0.7 | 0.62 |
| KLRD1 | 0.5 | 0.55 | 0.76 | 0.64 | 0.6125 |
| CCL4 | 0.56 | 0.55 | 0.66 | 0.66 | 0.6075 |
| IL17A | 0.58 | 0.64 | 0.66 | 0.54 | 0.605 |
| IFNG | 0.54 | 0.62 | 0.62 | 0.63 | 0.6025 |
| Glycolysis_SOLID (FGES) | 0.38 | 0.61 | 0.82 | 0.57 | 0.595 |
| CCL3 | 0.44 | 0.55 | 0.65 | 0.63 | 0.5675 |
| CSF2 | 0.41 | 0.67 | 0.65 | 0.52 | 0.5625 |
| ADPGK | 0.35 | 0.68 | 0.67 | 0.53 | 0.5575 |

TABLE 40
P-values of ROC-AUC of dynamic biomarkers from different sources
tested for the ability to differentiate patients with irAE vs patients
without irAE at each time point indicated and averaged.

| | | Internal 1 | Internal 1 | Internal 1 |
|---|---|---|---|---|
| | Internal 1 | (Melanoma), | (Melanoma), | (Melanoma), |
| | (Melanoma) | day 21 | day 42 | day 63 |
| Tregs (FGES) | 0.005 | 0.006 | 0.063 | 0.325 |
| IL2RA | 0.009 | 0.013 | 0.091 | 0.199 |
| FOXP3 | 0.001 | 0.016 | 0.031 | 0.960 |
| Activated_CD4_T_cells | 0.011 | 0.058 | 0.159 | 0.075 |
| (FGES) | | | | |
| IL1R1 | 0.116 | 0.312 | 0.022 | 0.016 |
| Tregs | 0.022 | 0.004 | 0.028 | 0.855 |
| KLRB1 | 0.317 | 0.082 | 0.126 | 0.040 |
| GATA3 | 0.002 | 0.107 | 0.481 | 0.701 |
| Effector_cells (FGES) | 0.281 | 0.327 | 0.047 | 0.224 |
| Cytotoxic_cell_inactivation | 0.443 | 0.456 | 0.020 | 0.199 |
| (FGES) | | | | |
| CD8_T_cells (FGES) | 0.269 | 0.312 | 0.184 | 0.342 |
| CCL5 | 0.330 | 0.341 | 0.159 | 0.250 |
| GADD45A | 0.649 | 0.128 | 0.505 | 0.055 |
| GZMB | 0.907 | 0.590 | 0.028 | 0.167 |
| FGF2 | 0.765 | 0.121 | 0.330 | 0.075 |
| KLRD1 | 0.990 | 0.673 | 0.043 | 0.211 |
| CCL4 | 0.541 | 0.694 | 0.242 | 0.167 |
| IL17A | 0.126 | 0.037 | 0.010 | 0.360 |
| IFNG | 0.687 | 0.312 | 0.391 | 0.237 |
| Glycolysis_SOLID (FGES) | 0.258 | 0.341 | 0.012 | 0.560 |
| CCL3 | 0.576 | 0.673 | 0.275 | 0.250 |
| CSF2 | 0.361 | 0.134 | 0.222 | 0.837 |
| ADPGK | 0.142 | 0.128 | 0.198 | 0.777 |

TABLE 41
Immune signature names and the associated
genes included in each signature.

| Signature | Genes |
|---|---|
| irAE Baseline | TNFSF14, TRAC, GZMK, NFKBID, CD5, ENO1, |
| Signature | CD69, CCR8, IKZF4, TBX21, ZAP70, PRF1, |
| | SERPIN89, TIGIT, CTLA4, TCF7, IL2RA, PGK1, |
| | GZMA, GZMB, GNLY, TNFRSF4, CD8B, |
| | NFATC1, PFKP, GPD2, GZMH, TRAT1, EOMES, |
| | LDHA, CD8A, IKZF2, LGALS9, BPGM, ICOS, |
| | KLRK1, KLRB1, CD27, NKG7, FOXP3, |
| | TNFRSF9, SIGLEC7, TNFRSF18, CCL5, CD28, |
| | FASLG, LAIR2, IL1R1, GPI, IFNG, KLRD1, CD4, |
| | CD40, CD40LG, LAIR1 |
| CTL Signature | CCL4, CD160, CTSW, EOMES, FASLG, FCRL6, |
| | FGFBP2, GNLY, GZMA, GZMB, GZMH, IL2RB, |
| | KLRB1, KLRC3, KLRD1, KLRF1, KLRG1, |
| | KLRK1, NCR3, NKG7, NMUR1, PRF1, PTGDR, |
| | PYHIN1, S1PR5, SAMD3, SH2D1B, SH2D2A, |
| | SLAMF7, TBX21, TIGIT, TRDC |
| CD4 Related | CACNA1I, CAMK4, CCR4, CCR7, CD28, CHMP7, |
| Signature | DUSP16, GATA3, IL2RA, ITPKB, KCNA3, |
| | LRIG1, MAL, PIK3C2B, RASA3, RCAN3, S1PR1, |
| | TESPA1, TNFRSF10A, TRABD2A, TRAF1, |
| | TRAT1, ZC3H12D, CD27, TCF7, ICOS, CD40LG, |
| | AQP3, CD5, CD6 |
| Glycolysis | BPGM, GSTO1, HAVCR2, HSP90B1, LDHA, |
| Signature | LGALS8, MAGT1, PDIA3, PDIA6, PGK1, PLIN1, |
| | PSMA6, SKIL, SPPL2A, YARS |
| Treg Signature | FOXP3, IKZF4, IKZF2, TNFRSF18, CCR8, IL2RA, |
| | CTLA4 |
| Antibody | CD38, IGHG1, TXNDCS, SDC1, TNFRSF17, |
| Secreting Cells | MZB1, PDIA4, CAV1, SPCS2, PRDX4, KDELR2 |
| Response | |

### Cellular Transcriptomic Signatures Predictor

Datasets with cell subtypes, such as CD8 T cells, were used, and only those containing or more samples were selected. For each dataset, the expression of all genes was correlated with a target gene, for example, LAG3. The mean and median correlations for each dataset were calculated. The 75th percentile was used to select genes: genes whose mean and median correlations exceed this threshold were selected.

Next, other datasets with cell subtypes, such as T cells, were considered, and those containing 5 or more samples were selected. Genes were filtered by previously obtained lists (mean and median), where expression is renormalized by the average sum, and correlation with the target gene is performed. Similar to the previous step, mean and median correlations were calculated for all genes, and those exceeding the 75th percentile were selected.

Then, datasets with whole blood or PBMCs sequencing were considered. Filtering by gene lists, expression renormalization, and calculation of correlations with the target gene were applied. For the final selection, genes that fall into both the average and median lists were selected. These genes were additionally filtered through open databases, such as STRING, to check their functional significance.

The final gene list is saved to form a signature, for example, for CD8 LAG3.

Signatures presented in Table 42 were collected according to the outlined framework, based on well-established literature biomarkers associated with adverse effects in CD4 and CD8 cells.

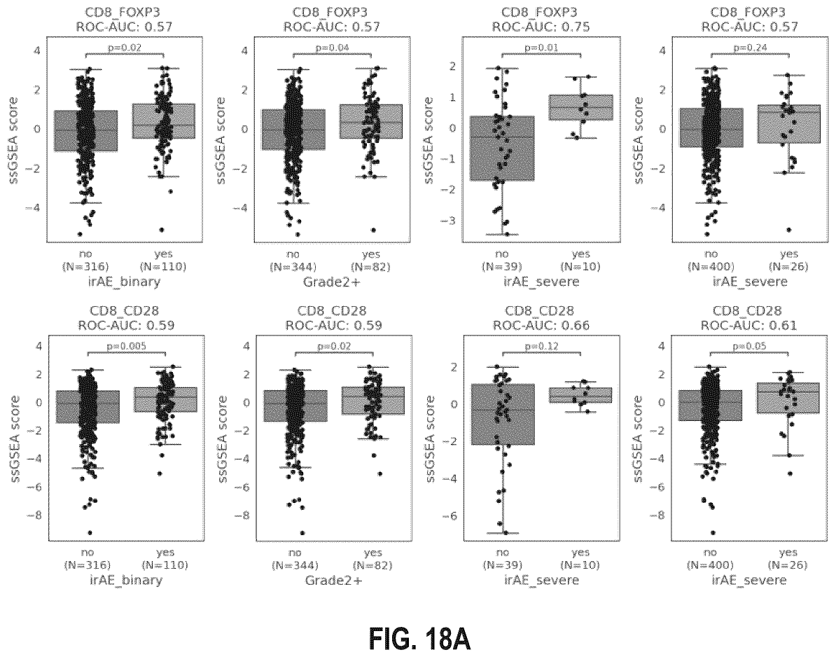

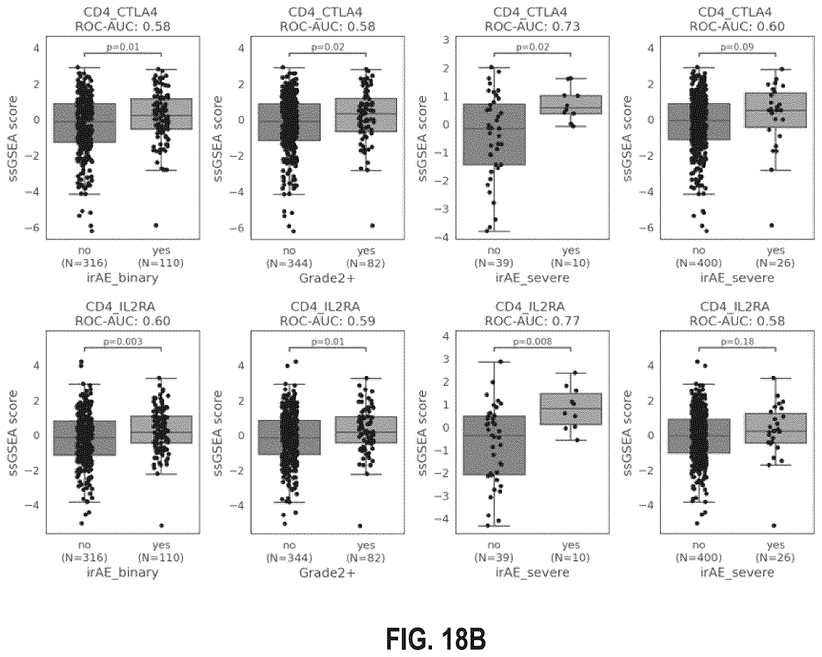

Using the framework described, multiple gene signatures were developed, as illustrated in the FIG. 18A and FIG. 18B, with examples including FOXP3, CD28, CTLA4, and IL2RA related signatures. These signatures have been shown to significantly distinguish patients with different levels of adverse effect severity across two independent cohorts, RADIOHEAD and MGH.

TABLE 42
Immune signature names and the associated genes included in each signature.

| Signature | 1 | 2 | 3 | 4 | 5 | 6 | 7 | 8 | 9 | 10 | 11 | 12 | 13 | 14 | 15 | 16 | 17 | 18 |
|---|---|---|---|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | | | | | | | | |
| [symbol] | [symbol] | [symbol] | [symbol] | [symbol] | [symbol] | [symbol] | [symbol] | [symbol] | [symbol] | [symbol] | [symbol] | [symbol] | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | | |
| [symbol] | [symbol] | [symbol] | [symbol] | [symbol] | [symbol] | [symbol] | [symbol] | [symbol] | | | | | | | | | | |
| [symbol] | [symbol] | [symbol] | [symbol] | [symbol] | [symbol] | [symbol] | [symbol] | [symbol] | [symbol] | | | | | | | | | |
| [symbol] | [symbol] | [symbol] | [symbol] | | | | | | | | | | | | | | | |
| [symbol] | [symbol] | [symbol] | [symbol] | [symbol] | [symbol] | [symbol] | [symbol] | [symbol] | [symbol] | [symbol] | [symbol] | | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | |
| [symbol] | [symbol] | [symbol] | [symbol] | [symbol] | [symbol] | [symbol] | [symbol] | | | | | | | | | | | |
| [symbol] | [symbol] | [symbol] | [symbol] | [symbol] | [symbol] | [symbol] | [symbol] | [symbol] | | | | | | | | | | |
| [symbol] | [symbol] | [symbol] | [symbol] | [symbol] | [symbol] | [symbol] | [symbol] | [symbol] | [symbol] | [symbol] | | | | | | | | |
| [symbol] | [symbol] | [symbol] | [symbol] | | | | | | | | | | | | | | | |
| [symbol] | [symbol] | [symbol] | [symbol] | [symbol] | [symbol] | [symbol] | [symbol] | [symbol] | [symbol] | | | | | | | | | |
| [symbol] | [symbol] | [symbol] | [symbol] | [symbol] | [symbol] | [symbol] | [symbol] | [symbol] | [symbol] | [symbol] | [symbol] | [symbol] | | | | | | |
| [symbol] | [symbol] | [symbol] | [symbol] | [symbol] | [symbol] | [symbol] | [symbol] | [symbol] | [symbol] | | | | | | | | | |
| [symbol] | [symbol] | [symbol] | [symbol] | [symbol] | [symbol] | [symbol] | [symbol] | | | | | | | | | | | |
| [symbol] | [symbol] | [symbol] | [symbol] | [symbol] | [symbol] | [symbol] | | | | | | | | | | | | |
| [symbol] | [symbol] | [symbol] | [symbol] | [symbol] | [symbol] | [symbol] | [symbol] | [symbol] | [symbol] | [symbol] | | | | | | | | |
| [symbol] | [symbol] | [symbol] | [symbol] | [symbol] | [symbol] | [symbol] | [symbol] | [symbol] | [symbol] | [symbol] | [symbol] | [symbol] | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | | | | | |
| [symbol] | [symbol] | [symbol] | [symbol] | [symbol] | [symbol] | [symbol] | [symbol] | [symbol] | [symbol] | | | | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | | | |
| [symbol] | [symbol] | [symbol] | | | | | | | | | | | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | | | | |
| [symbol] | [symbol] | [symbol] | [symbol] | [symbol] | [symbol] | [symbol] | [symbol] | | | | | | | | | | | |
| [symbol] | [symbol] | [symbol] | [symbol] | | | | | | | | | | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | | | |
| [symbol] | [symbol] | [symbol] | [symbol] | [symbol] | [symbol] | | | | | | | | | | | | | |
| [symbol] | [symbol] | [symbol] | [symbol] | [symbol] | [symbol] | [symbol] | [symbol] | [symbol] | [symbol] | | | | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | | | | | |

[symbol]

indicates data missing or illegible when filed

### Immunotypes

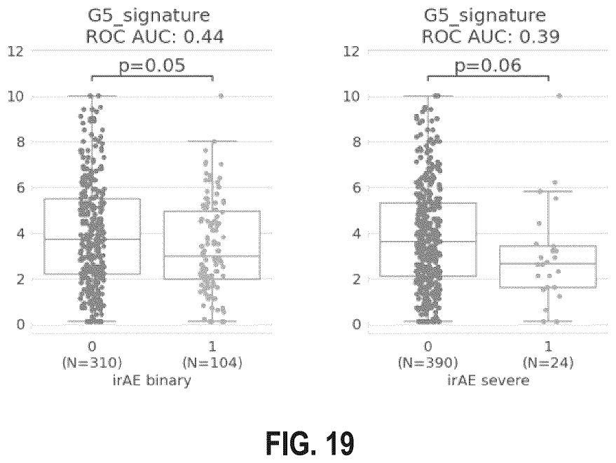

Immune type G5 aids in the stratification of patients with potential adverse effects, providing a valuable tool for risk assessment. A detailed description of this immune profile is available (PMID: 38744245), offering further insights into its characteristics and relevance in patient stratification. FIG. 19 shows boxplots of the immunotype signature G5 from the RADIOHEAD dataset, illustrating the separation between patients with and without adverse effects.

### Embeddings

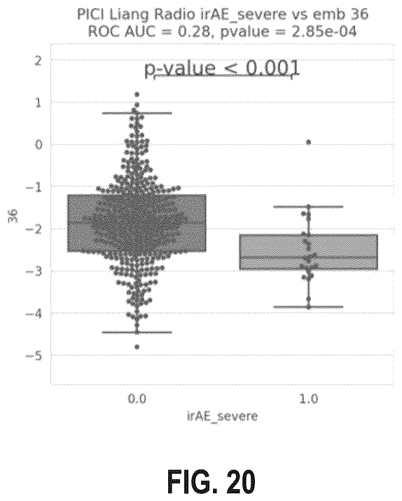

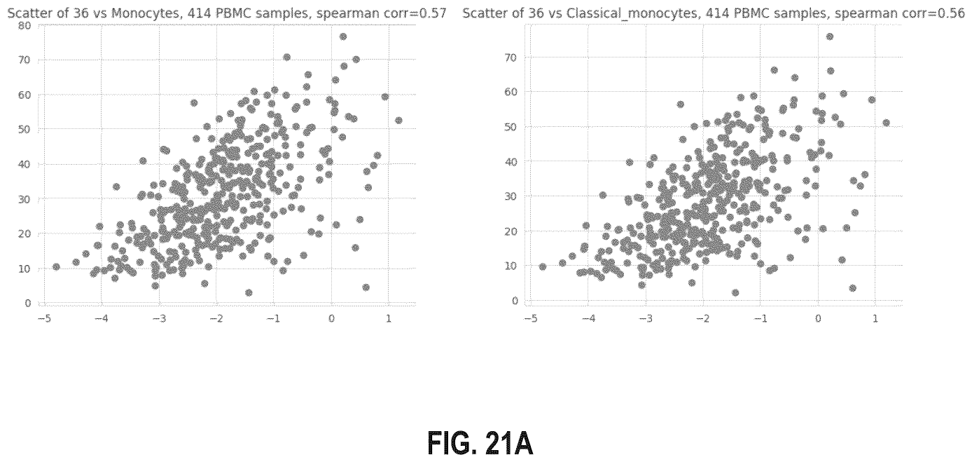

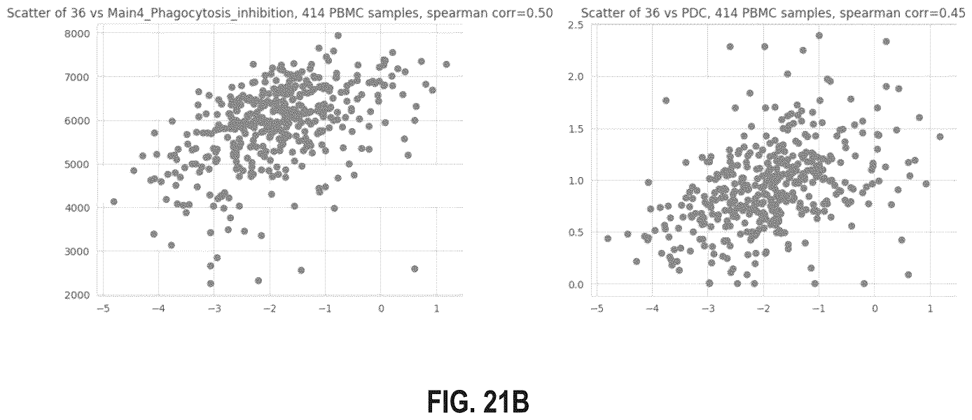

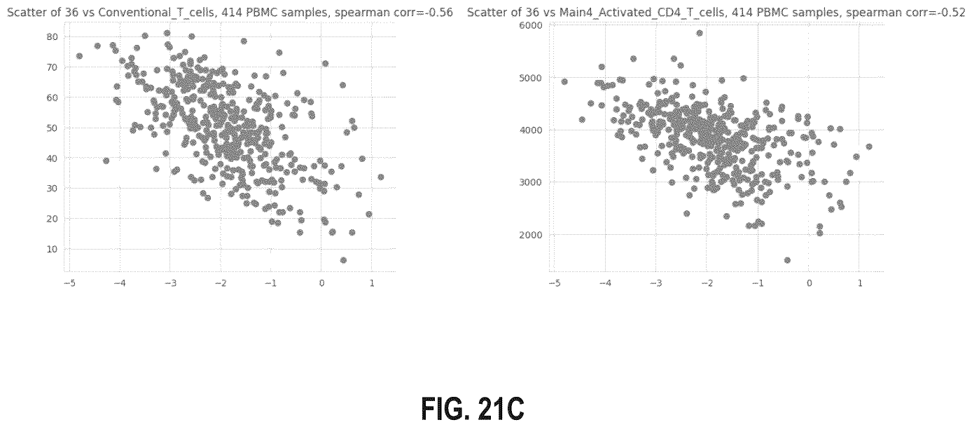

An autoencoder was trained on over 30,000 open-source blood and sorted cell samples, compressing them into a 64-dimensional space. FIG. 20 displays a box plot of the embedding, which demonstrates the most significant separation of patients with adverse effects, adjusted for multiple comparisons. Further analysis revealed that this embedding correlates with the biological features shown in FIGS. 21A-21C, suggesting that these features characterize the embedding and play a role in the detection of adverse effects,

### 3. Cytometry

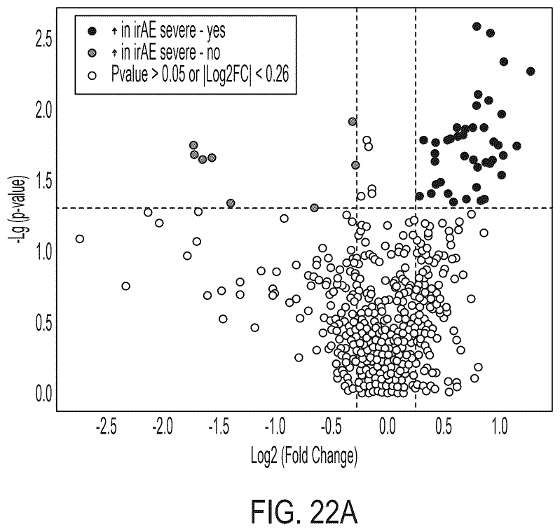

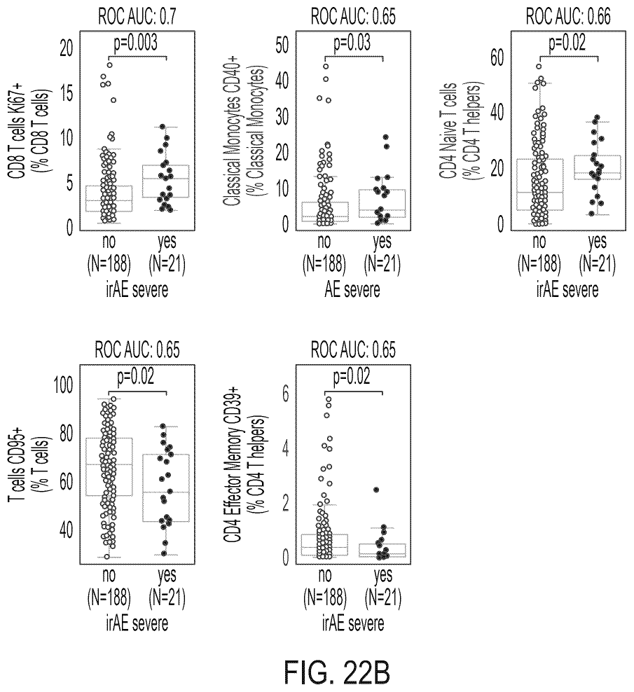

A preliminary differential analysis of flow cytometry data was performed to identify blood cell populations associated with severe irAEs (FIG. 22A). Each point on the volcano plot represents an individual immune population, expressed as a percentage relative to its parent population. The x-axis shows the log₂ fold change of medians between groups, and the y-axis displays the −log₁₀(p-value) from the Mann-Whitney U test. Populations with a fold change greater than 1.2 and a p-value below 0.05 were considered significantly different, corresponding to at least a 20% difference in median abundance between groups. Populations increased in patients with severe irAEs included CD8⁺ T cells Ki67⁺, classical monocytes CD40⁺, and naïve CD4⁺ T cells, suggesting enhanced immune activation and proliferation. In contrast, CD95⁺ T cells and CD4⁺ effector memory T cells CD39⁺ were more abundant in patients without irAEs, reflecting a more regulated immune state. Representative populations (CD8⁺ Ki67⁺ T cells, classical monocytes CD40⁺, naïve CD4⁺ T cells, CD95⁺ T cells, and CD4⁺ effector memory CD39⁺ T cells) are shown as boxplots. (FIG. 22B).

### 4. Benchmark

Publicly available predictors of adverse effects that were accessible for benchmarking purposes in testing were gathered. These predictors, presented in Table 43, encompass a wide range of methodologies and biological targets, providing a comprehensive basis for comparative analysis across different datasets.

TABLE 43
Publicly available adverse effect predictors
with associated quality metrics.

| PMID | ROC_AUC_orig | NPV_orig | R\hat{ }2_orig |
|---|---|---|---|
| 38743882 | NA | 0.97 | 0.16 |
| 38743882 | NA | 0.98 | 0.21 |
| 37292751 | 0.66 | NA | NA |
| 37292751 | 0.65 | NA | NA |
| 35027754 | 0.8 to 0.9 | | |

### Example Aspects

Some aspects provide for a method for predicting whether a subject will experience an immune-related adverse event (irAE) in response to administration of an immune checkpoint inhibitor (ICI) therapy to the subject, the method comprising: using at least one processor to perform: obtaining healthcare data for the subject, the healthcare data comprising at least two of: clinical data for the subject, RNA sequencing data for the subject, and immune receptor data for the subject; determining, using at least some of the healthcare data, a likelihood that the subject will experience the irAE in response to administration of the ICI therapy, the determining comprising: performing at least two of: (a) processing the clinical data for the subject using a first machine learning (ML) model to output a first likelihood that the subject will experience the irAE in response to administration of the ICI therapy, wherein the first ML model is trained to predict, from clinical data for a particular subject, a likelihood that the particular subject will experience the irAE in response to administration of the ICI therapy, (b) processing the RNA sequencing data for the subject using a second ML model to output a second likelihood that the subject will experience the irAE in response to administration of the ICI therapy, wherein the second ML model is trained to predict, from RNA sequencing data for a particular subject, a likelihood that the particular subject will experience the irAE in response to administration of the ICI therapy, and (c) processing the immune receptor data for the subject using a third ML model to output a third likelihood that the subject will experience the irAE in response to administration of the ICI therapy, wherein the third ML model is trained to predict, from immune receptor data for a particular subject, a likelihood that the particular subject will experience the irAE in response to administration of the ICI therapy; and processing two or more of the first, second, and third likelihoods using a fourth ML model trained to predict the likelihood that the subject will experience the irAE in response to administration of the ICI therapy from one or more of the likelihoods that the subject will experience the irAE determined using two or more of first-, second- and third-ML models; and outputting the likelihood that the subject will experience the irAE in response to administration of the ICI therapy.

Some aspects provide for a system, comprising: at least one processor; and at least one non-transitory computer-readable storage medium storing processor-executable instructions that, when executed by the at least one processor, cause the at least one processor to perform a method for predicting whether a subject will experience an immune-related adverse event (irAE) in response to administration of an immune checkpoint inhibitor (ICI) therapy to the subject, the method comprising: obtaining healthcare data for the subject, the healthcare data comprising at least two of: clinical data for the subject, RNA sequencing data for the subject, and immune receptor data for the subject; determining, using at least some of the healthcare data, a likelihood that the subject will experience the irAE in response to administration of the ICI therapy, the determining comprising: performing at least two of: (a) processing the clinical data for the subject using a first machine learning (ML) model to output a first likelihood that the subject will experience the irAE in response to administration of the ICI therapy, wherein the first ML model is trained to predict, from clinical data for a particular subject, a likelihood that the particular subject will experience the irAE in response to administration of the ICI therapy, (b) processing the RNA sequencing data for the subject using a second ML model to output a second likelihood that the subject will experience the irAE in response to administration of the ICI therapy, wherein the second ML model is trained to predict, from RNA sequencing data for a particular subject, a likelihood that the particular subject will experience the irAE in response to administration of the ICI therapy, and (c) processing the immune receptor data for the subject using a third ML model to output a third likelihood that the subject will experience the irAE in response to administration of the ICI therapy, wherein the third ML model is trained to predict, from immune receptor data for a particular subject, a likelihood that the particular subject will experience the irAE in response to administration of the ICI therapy; and processing two or more of the first, second, and third likelihoods using a fourth ML model trained to predict the likelihood that the subject will experience the irAE in response to administration of the ICI therapy from one or more of the likelihoods that the subject will experience the irAE determined using two or more of first-, second- and third-ML models; and outputting the likelihood that the subject will experience the irAE in response to administration of the ICI therapy.

Some aspects provide for a least one non-transitory computer-readable storage medium storing processor-executable instructions that, when executed by at least one processor, cause the at least one processor to perform a method for predicting whether a subject will experience an immune-related adverse event (irAE) in response to administration of an immune checkpoint inhibitor (ICI) therapy to the subject, the method comprising: obtaining healthcare data for the subject, the healthcare data comprising at least two of: clinical data for the subject, RNA sequencing data for the subject, and immune receptor data for the subject; determining, using at least some of the healthcare data, a likelihood that the subject will experience the irAE in response to administration of the ICI therapy, the determining comprising: performing at least two of: (a) processing the clinical data for the subject using a first machine learning (ML) model to output a first likelihood that the subject will experience the irAE in response to administration of the ICI therapy, wherein the first ML model is trained to predict, from clinical data for a particular subject, a likelihood that the particular subject will experience the irAE in response to administration of the ICI therapy, (b) processing the RNA sequencing data for the subject using a second ML model to output a second likelihood that the subject will experience the irAE in response to administration of the ICI therapy, wherein the second ML model is trained to predict, from RNA sequencing data for a particular subject, a likelihood that the particular subject will experience the irAE in response to administration of the ICI therapy, and (c) processing the immune receptor data for the subject using a third ML model to output a third likelihood that the subject will experience the irAE in response to administration of the ICI therapy, wherein the third ML model is trained to predict, from immune receptor data for a particular subject, a likelihood that the particular subject will experience the irAE in response to administration of the ICI therapy; and processing two or more of the first, second, and third likelihoods using a fourth ML model trained to predict the likelihood that the subject will experience the irAE in response to administration of the ICI therapy from one or more of the likelihoods that the subject will experience the irAE determined using two or more of first-, second- and third-ML models; and outputting the likelihood that the subject will experience the irAE in response to administration of the ICI therapy.

Embodiments of any of the above aspects may have one or more of the following features.

Some embodiments further comprise: outputting a recommendation to administer the ICI therapy to the subject when the likelihood that the subject will experience the irAE is less than or equal to a threshold.

Some embodiments further comprise: administering the ICI therapy to the subject when the likelihood that the subject will experience the irAE is less than or equal to the threshold.

Some embodiments further comprise: when the likelihood that the subject will experience the irAE is greater than or equal to a threshold: generating, using the RNA sequencing data for the subject, human leukocyte antigen (HLA) input features indicative of HLA alleles present in a genome of the subject; and processing the HLA input features using an ML model for predicting inflammatory bowel disease (IBD) to output a likelihood that the subject will develop IBD in response to the administration of the ICI therapy, wherein the ML model for predicting IBD is trained to predict, from HLA input features for a particular subject, a likelihood that the particular subject will develop IBD.

In some embodiments, generating the HLA input features using the RNA sequencing data for the subject comprises generating: (i) a first input feature indicative of a number of HLA alleles present in the genome of the subject that are associated with a risk of IBD, (ii) a second input feature indicative of a number of HLA alleles present in the genome of the subject that are not associated with the risk of IBD, and (iii) one or more third input features, each of the one or more third input features indicative of a respective HLA allele present in the genome of the subject.

In some embodiments, the ML model for predicting IBD is a gradient-boosted decision tree model implemented using CatBoost, XGBoost, or LightGBM.

In some embodiments, the healthcare data comprises the clinical data for the subject, and determining, using the at least some of the healthcare data, the likelihood that the subject will experience the irAE in response to administration of the ICI therapy comprises: processing the clinical data for the subject using the first ML model to output the first likelihood that the subject will experience the irAE in response to administration of the ICI therapy.

In some embodiments, the clinical data for the subject indicates: age, gender, diagnosis, disease stage, therapy type, and metastatic status for the subject.

In some embodiments, the first ML model is a random forest model.

In some embodiments, the healthcare data comprises the RNA sequencing data for the subject, and determining, using the at least some of the healthcare data, the likelihood that the subject will experience the irAE in response to administration of the ICI therapy comprises: processing the RNA sequencing data for the subject using the second ML model to output the second likelihood that the subject will experience the irAE in response to administration of the ICI therapy.

In some embodiments, processing the RNA sequencing data for the subject using the second ML model comprises: determining a plurality of immune signatures using the RNA sequencing data for the subject, each of the plurality of immune signatures representing RNA expression levels for at least some genes in a respective gene group of a plurality of gene groups; and processing the plurality of immune signatures using the second ML model to obtain the second likelihood that the subject will experience the irAE in response to administration of the ICI therapy.

In some embodiments, the RNA sequencing data for the subject indicates RNA expression levels for at least some genes in each group of at least some of the plurality of gene groups, the plurality of gene groups comprising: LDHB glycolysis signature: LDHB, DGKA, GCNT4, TBC1D4, ETS1; Treg and T-cell activation signature: ABCC1, ARID5B, BCL2, BIRC3, CCND2, CCR4, CD2, CD28, CISH, CTLA4, FAS, FOXP3, GATA3, ICOS, IL12RB2, IL2RA, IL2RB, LTA, MAF, MAP3K14, OPTN, P2RY10, PIM2, POU2AF1, RTKN2, SLAMF1, SOCS1, SOCS2, TIGIT, TRADD, TRAF1, TRAF2; irAE-associated T-cell signature: TNFRSF4, CD28, KLRB1, TNFRSF18, CD40, IFNG, TRAT1, EOMES, CD69, CCR8, GZMA, TIGIT, TNFRSF9, ZAP70, TCF7, KLRK1, ICOS, CD8B, FASLG, CD27, IKZF2, PRF1, GZMB, LAIR2, GZMK, CCL5, CD5, GZMH, CD8A, PFKP, CD40LG, KLRD1, TBX21, NKG7, GNLY, CTLA4, TRAC; Treg signature: FOXP3, CTLA4, IL2RA, CCR8, IKZF4, IKZF2, RTKN2, CCR4, FAS; CD4-related signature: CD28, TCF7, IL2RA, CHMP7, CCR4, CAMK4, S1PR1, DUSP16, MAL, AQP3, CCR7, RASA3, CD40LG, GATA3, KCNA3, RCAN3, ZC3H12D, CD6, LRIG1, TRAF1, TRAT1, CD27, TRABD2A, TESPA1, ICOS, CACNA11, ITPKB, PIK3C2B, TNFRSFIOA, CD5; Antigen specific T-cell activation: TESPA1, SIRPG, CD3G, SLAMF6, CD27, LCK, IKZF3, FCMR, LDLRAP1, LTB, EPB41, LAT, CD3D, PTPRCAP, ADD3, CD2, MAP4K1, SIT1, ESYT1, UBASH3A, TRAF3IP3, CD3E, SAMD3, THEMIS, LIME1, LY9, GRAP, SKAP1, TCF7, ITM2A, KLRG1; Hypoxia factors signature: FUT11, NDRG1, EPAS1, CA9, LDHA, LOX, SLC2A1, P4HA1, CA12, HK2, PDK1, PGK1, TPI1, ALDOA, PFKFB3; LDHA glycolysis signature: HAVCR2, PGK1, LDHA, PSMA6, BPGM, PDIA3, PDIA6, PLIN2, SPPL2A, LGALS8, YARS, HSP90B1, MAGT1, SKIL, GSTO1; Platelet signature: ITGA2B, ITGB3, SELP, MPL, GP1BA, GP1BB, TUBB1; TNF signaling-associated signature: AREG, EREG, LAMB3, PLAU, PTX3; Myeloid suppression signature: TGFB2, IL10, CCL24, CXCL8, S100A12, EBI3, MSR1, PTGS2, SLC11A1, TREM1, PLAUR; M2 polarization signature: TGFB2, TGFB3, IL10, CCL18, IL33, CCL24; and Autophagy signature ATG12, ATG9A, TFEB, RB1CC1, MAP1LC3B, GABARAPL2, ATG4B, ATG7, GABARAP, VMP1, ATG14, GABARAPL1, ATG13, NBR1.

In some embodiments, determining the plurality of immune signatures using the RNA sequencing data for the subject comprises: determining gene group scores for respective gene groups in the at least some of the plurality of gene groups using the RNA expression levels.

In some embodiments, processing the RNA sequencing data for the subject using the second ML model comprises: determining, using the RNA sequencing data for the subject, (i) a proportion of classical dendritic cells (cDCs) to dendritic cells and (ii) a proportion of memory T cells to T cells; and processing (i) the proportion of cDCs to dendritic cells, (ii) the proportion of memory T cells to T cells, and (iii) the plurality of immune signatures, using the second ML model to output the second likelihood that the subject will experience the irAE in response to administration of the ICI therapy.

In some embodiments, the healthcare data further comprises immune cell data, and wherein processing the RNA sequencing data using the second ML model further comprises: determining, using the immune cell data, (i) a proportion of classical dendritic cells (cDCs) to dendritic cells and (ii) a proportion of memory T cells to T cells; and processing (i) the proportion of cDCs to dendritic cells, (ii) the proportion of memory T cells to T cells, and (iii) the plurality of immune signatures, using the second ML model to output the second likelihood that the subject will experience the irAE in response to administration of the ICI therapy.

In some embodiments, the second ML model is a logistic regression model.

In some embodiments, the healthcare data comprises the immune receptor data for the subject, and determining, using the at least some of the healthcare data, the likelihood that the subject will experience the irAE in response to administration of the ICI therapy comprises: processing the immune receptor data for the subject using the third ML model to output the third likelihood that the subject will experience the irAE in response to administration of the ICI therapy.

In some embodiments, the immune receptor data comprises B cell receptor sequence data and T cell receptor sequence data.

In some embodiments, processing the immune receptor data using the third ML model comprises: determining, using the B cell receptor sequence data, a value indicative of B cell receptor diversity; determining, using the T cell receptor sequence data, a value indicative of T cell receptor diversity; determining, using the B cell receptor data, a proportion of a number of IgH clonotypes having a particular variable gene with respect to a total number of IgH clonotypes; and processing, using the third ML model, the value indicative of B cell receptor diversity, the value indicative of T cell receptor diversity, and the proportion of the number of IgH clonotypes associated with the particular variable gene with respect to the total number of IgH clonotypes.

In some embodiments, the value indicative of the B cell receptor diversity and the value indicative of the T cell receptor diversity are computed according to:

$$- \frac{1}{N} {\sum }_{n = 1}^{N} {\sum }_{i = 1}^{{s}_{N}} {p}_{i , N} ln ( {p}_{i , N} )$$

where: N represents a number of receptor chains; s_{N} represents a number of clonotypes for a particular receptor chain, and p_{i,N} represents a proportion of a frequency of a particular clonotype with respect to a frequency of all clonotypes for the particular receptor chain.

In some embodiments, the particular variable gene is IgHV4-34.

In some embodiments, the third ML model is a logistic regression model.

In some embodiments, the healthcare data comprises the clinical data for the subject, the RNA sequencing data for the subject, and the immune receptor data for the subject, and determining, using the at least some of the healthcare data, the likelihood that the subject will experience the irAE in response to administration of the ICI therapy comprises: performing: (a) processing the clinical data for the subject using the first ML model to output the first likelihood that the subject will experience the irAE in response to administration of the ICI therapy; (b) processing the RNA sequencing data for the subject using the second ML model to output the second likelihood that the subject will experience the irAE in response to administration of the ICI therapy; and (c) processing the immune receptor data for the subject using the third ML model to output the third likelihood that the subject will experience the irAE in response to administration of the ICI therapy.

In some embodiments, the fourth ML model is a logistic regression model.

In some embodiments, processing two or more of the first, second, and third likelihoods using the fourth ML model trained to predict the likelihood that the subject will experience the irAE in response to administration of the ICI therapy comprises processing the first, second, and third likelihoods using the fourth ML model trained to predict the likelihood that the subject will experience the irAE in response to administration of the ICI therapy.

Some aspects provide for a method for predicting whether a subject will experience an immune-related adverse event (irAE) in response to administration of an immune checkpoint inhibitor (ICI) therapy to the subject, the method comprising: using at least one processor to perform: determining, using RNA sequencing data and/or immune cell data, (i) a proportion of classical dendritic cells (cDCs) to dendritic cells and (ii) a proportion of memory T cells to T cells; determining a plurality of immune signatures using the RNA sequencing data, each of the plurality of immune signatures representing RNA expression levels for at least some genes in a respective gene group of a plurality of gene groups; processing (i) the proportion of cDCs to dendritic cells, (ii) the proportion of memory T cells to T cells, and (iii) the plurality of immune signatures, using a machine learning (ML) model trained to predict a likelihood that the subject will experience the irAE in response to administration of the ICI therapy from (i) the proportion of cDCs to dendritic cells, (ii) the proportion of memory T cells to T cells, and (iii) the plurality of immune signatures; and outputting the likelihood that the subject will experience the irAE in response to administration of the ICI therapy.

Some aspects provide for a system, comprising: at least one processor; and at least one non-transitory computer-readable storage medium storing processor-executable instructions that, when executed by the at least one processor, cause the at least one processor to perform a method for predicting whether a subject will experience an immune-related adverse event (irAE) in response to administration of an immune checkpoint inhibitor (ICI) therapy to the subject, the method comprising: determining, using RNA sequencing data and/or immune cell data, (i) a proportion of classical dendritic cells (cDCs) to dendritic cells and (ii) a proportion of memory T cells to T cells; determining a plurality of immune signatures using the RNA sequencing data, each of the plurality of immune signatures representing RNA expression levels for at least some genes in a respective gene group of a plurality of gene groups; processing (i) the proportion of cDCs to dendritic cells, (ii) the proportion of memory T cells to T cells, and (iii) the plurality of immune signatures, using a machine learning (ML) model trained to predict a likelihood that the subject will experience the irAE in response to administration of the ICI therapy from (i) the proportion of cDCs to dendritic cells, (ii) the proportion of memory T cells to T cells, and (iii) the plurality of immune signatures; and outputting the likelihood that the subject will experience the irAE in response to administration of the ICI therapy.

Some aspects provide for at least one non-transitory computer-readable storage medium storing processor-executable instructions that, when executed by at least one processor, cause the at least one processor to perform a method for predicting whether a subject will experience an immune-related adverse event (irAE) in response to administration of an immune checkpoint inhibitor (ICI) therapy to the subject, the method comprising: determining, using RNA sequencing data and/or immune cell data, (i) a proportion of classical dendritic cells (cDCs) to dendritic cells and (ii) a proportion of memory T cells to T cells; determining a plurality of immune signatures using the RNA sequencing data, each of the plurality of immune signatures representing RNA expression levels for at least some genes in a respective gene group of a plurality of gene groups; processing (i) the proportion of cDCs to dendritic cells, (ii) the proportion of memory T cells to T cells, and (iii) the plurality of immune signatures, using a machine learning (ML) model trained to predict a likelihood that the subject will experience the irAE in response to administration of the ICI therapy from (i) the proportion of cDCs to dendritic cells, (ii) the proportion of memory T cells to T cells, and (iii) the plurality of immune signatures; and outputting the likelihood that the subject will experience the irAE in response to administration of the ICI therapy.

Embodiments of any of the above aspects may have one or more of the following features.

In some embodiments, determining, using the RNA sequencing data and/or the immune cell data, (i) the proportion of cDCs to dendritic cells and (ii) the proportion of memory T cells to T cells comprises: determining, using the RNA sequencing data, (i) the proportion of cDCs to dendritic cells and (ii) the proportion of memory T cells to T cells.

In some embodiments, determining, using the RNA sequencing data and/or the immune cell data, (i) the proportion of cDCs to dendritic cells and (ii) the proportion of memory T cells to T cells comprises: determining, using the immune cell data, (i) the proportion of cDCs to dendritic cells and (ii) the proportion of memory T cells to T cells.

In some embodiments, the RNA sequencing data indicates RNA expression levels for at least some genes in each group of at least some of the plurality of gene groups, the plurality of gene groups comprising: LDHB glycolysis signature: LDHB, DGKA, GCNT4, TBC1D4, ETS1; Treg and T-cell activation signature: ABCC1, ARID5B, BCL2, BIRC3, CCND2, CCR4, CD2, CD28, CISH, CTLA4, FAS, FOXP3, GATA3, ICOS, IL12RB2, IL2RA, IL2RB, LTA, MAF, MAP3K14, OPTN, P2RY10, PIM2, POU2AF1, RTKN2, SLAMF1, SOCS1, SOCS2, TIGIT, TRADD, TRAF1, TRAF2; irAE-associated T-cell signature: TNFRSF4, CD28, KLRB1, TNFRSF18, CD40, IFNG, TRAT1, EOMES, CD69, CCR8, GZMA, TIGIT, TNFRSF9, ZAP70, TCF7, KLRK1, ICOS, CD8B, FASLG, CD27, IKZF2, PRF1, GZMB, LAIR2, GZMK, CCL5, CD5, GZMH, CD8A, PFKP, CD40LG, KLRD1, TBX21, NKG7, GNLY, CTLA4, TRAC; Treg signature: FOXP3, CTLA4, IL2RA, CCR8, IKZF4, IKZF2, RTKN2, CCR4, FAS; CD4-related signature: CD28, TCF7, IL2RA, CHMP7, CCR4, CAMK4, SIPRI, DUSP16, MAL, AQP3, CCR7, RASA3, CD40LG, GATA3, KCNA3, RCAN3, ZC3H12D, CD6, LRIG1, TRAF1, TRAT1, CD27, TRABD2A, TESPA1, ICOS, CACNA11, ITPKB, PIK3C2B, TNFRSF10A, CD5; Antigen specific T-cell activation: TESPA1, SIRPG, CD3G, SLAMF6, CD27, LCK, IKZF3, FCMR, LDLRAP1, LTB, EPB41, LAT, CD3D, PTPRCAP, ADD3, CD2, MAP4K1, SIT1, ESYT1, UBASH3A, TRAF3IP3, CD3E, SAMD3, THEMIS, LIME1, LY9, GRAP, SKAP1, TCF7, ITM2A, KLRG1; Hypoxia factors signature: FUT11, NDRG1, EPAS1, CA9, LDHA, LOX, SLC2A1, P4HA1, CA12, HK2, PDK1, PGK1, TPI1, ALDOA, PFKFB3; LDHA glycolysis signature: HAVCR2, PGK1, LDHA, PSMA6, BPGM, PDIA3, PDIA6, PLIN2, SPPL2A, LGALS8, YARS, HSP90B1, MAGT1, SKIL, GSTO1; Platelet signature: ITGA2B, ITGB3, SELP, MPL, GP1BA, GP1BB, TUBB1; TNF signaling-associated signature: AREG, EREG, LAMB3, PLAU, PTX3; Myeloid suppression signature: TGFB2, IL10, CCL24, CXCL8, S100A12, EBI3, MSR1, PTGS2, SLC11A1, TREM1, PLAUR; M2 polarization signature: TGFB2, TGFB3, IL10, CCL18, IL33, CCL24; and Autophagy signature ATG12, ATG9A, TFEB, RB1CC1, MAP1LC3B, GABARAPL2, ATG4B, ATG7, GABARAP, VMP1, ATG14, GABARAPL1, ATG13, NBR1.

In some embodiments, determining the plurality of immune signatures using the RNA sequencing data comprises: determining gene group scores for respective gene groups in the at least some of the plurality of gene groups using the RNA expression levels.

In some embodiments, the ML model is a logistic regression model.

Some embodiments further comprise: outputting a recommendation to administer the ICI therapy to the subject when the likelihood that the subject will experience the irAE is less than or equal to a threshold.

Some embodiments further comprise: administering the ICI therapy to the subject when the likelihood that the subject will experience the irAE is less than or equal to the threshold.

Some embodiments further comprise: when the likelihood that the subject will experience the irAE is greater than or equal to a threshold: generating, using the RNA sequencing data, human leukocyte antigen (HLA) input features indicative of HLA alleles present in a genome of the subject; and processing the HLA input features using an ML model for predicting inflammatory bowel disease (IBD) to output a likelihood that the subject will develop IBD in response to the administration of the ICI therapy, wherein the ML model for predicting IBD is trained to predict, from HLA input features for particular subject, a likelihood that the particular subject will develop IBD.

In some embodiments, generating the HLA input features using the RNA sequencing data comprises generating: (i) a first input feature indicative of a number of HLA alleles present in the genome of the subject that are associated with a risk of IBD, (ii) a second input feature indicative of a number of HLA alleles present in the genome of the subject that are not associated with the risk of IBD, and (iii) one or more third input features, each of the one or more third input features indicative of a respective HLA allele present in the genome of the subject.

In some embodiments, the ML model for predicting IBD is a gradient-boosted decision tree model implemented using CatBoost, XGBoost, or LightGBM.

Some aspects provide for a method for predicting whether a subject will develop inflammatory bowel disease (IBD) in response to administration of an immune checkpoint inhibitor (ICI) therapy, the method comprising: using at least one processor to perform: obtaining sequencing data for the subject, the sequencing data indicating whether a plurality of human leukocyte antigen (HLA) alleles are present in a genome of the subject, the plurality of HLA alleles comprising: (i) a first set of HLA alleles associated with a risk of IBD, and (ii) a second set of HLA alleles not associated with a risk of IBD; providing, as input to a machine learning (ML) model, a plurality of input features including: (i) a first input feature indicative of a number of HLA alleles in the first set of HLA alleles associated with the risk of IBD that are present in the genome of the subject, (ii) a second input feature indicative of a number of HLA alleles in the second set of HLA alleles not associated with the risk of IBD that are present in the genome of the subject, and (iii) one or more third input features indicative of HLA alleles present in the genome of the subject; processing the input using the ML model to output a likelihood that the subject will develop IBD in response to the administration of the ICI therapy, wherein the ML model is trained to predict the likelihood that the subject will develop IBD in response to administration of the ICI therapy from (i) the first input feature, (ii) the second input feature, and (iii) the one or more third input features; and outputting the likelihood that the subject will develop IBD in response to the administration of the ICI therapy.

Some aspects provide for a system, comprising: at least one processor; and at least one non-transitory computer-readable storage medium storing processor-executable instructions that, when executed by the at least one processor, cause the at least one processor to perform a method for predicting whether a subject will develop inflammatory bowel disease (IBD) in response to administration of an immune checkpoint inhibitor (ICI) therapy, the method comprising: obtaining sequencing data for the subject, the sequencing data indicating whether a plurality of human leukocyte antigen (HLA) alleles are present in a genome of the subject, the plurality of HLA alleles comprising: (i) a first set of HLA alleles associated with a risk of IBD, and (ii) a second set of HLA alleles not associated with a risk of IBD; providing, as input to a machine learning (ML) model, a plurality of input features including: (i) a first input feature indicative of a number of HLA alleles in the first set of HLA alleles associated with the risk of IBD that are present in the genome of the subject, (ii) a second input feature indicative of a number of HLA alleles in the second set of HLA alleles not associated with the risk of IBD that are present in the genome of the subject, and (iii) one or more third input features indicative of HLA alleles present in the genome of the subject; processing the input using the ML model to output a likelihood that the subject will develop IBD in response to the administration of the ICI therapy, wherein the ML model is trained to predict the likelihood that the subject will develop IBD in response to administration of the ICI therapy from (i) the first input feature, (ii) the second input feature, and (iii) the one or more third input features; and outputting the likelihood that the subject will develop IBD in response to the administration of the ICI therapy.

Some aspects provide for at least one non-transitory computer-readable storage medium storing processor-executable instructions that, when executed by at least one processor, cause the at least one processor to perform a method for predicting whether a subject will develop inflammatory bowel disease (IBD) in response to administration of an immune checkpoint inhibitor (ICI) therapy, the method comprising: obtaining sequencing data for the subject, the sequencing data indicating whether a plurality of human leukocyte antigen (HLA) alleles are present in a genome of the subject, the plurality of HLA alleles comprising: (i) a first set of HLA alleles associated with a risk of IBD, and (ii) a second set of HLA alleles not associated with a risk of IBD; providing, as input to a machine learning (ML) model, a plurality of input features including: (i) a first input feature indicative of a number of HLA alleles in the first set of HLA alleles associated with the risk of IBD that are present in the genome of the subject, (ii) a second input feature indicative of a number of HLA alleles in the second set of HLA alleles not associated with the risk of IBD that are present in the genome of the subject, and (iii) one or more third input features indicative of HLA alleles present in the genome of the subject; processing the input using the ML model to output a likelihood that the subject will develop IBD in response to the administration of the ICI therapy, wherein the ML model is trained to predict the likelihood that the subject will develop IBD in response to administration of the ICI therapy from (i) the first input feature, (ii) the second input feature, and (iii) the one or more third input features; and outputting the likelihood that the subject will develop IBD in response to the administration of the ICI therapy.

Embodiments of any of the above aspects may have one or more of the following features.

In some embodiments, the plurality of HLA alleles comprise at least some of: C*02:205Q, C*03:04, C*06:201, DMA*01:01, DMA*01:05, DMA*01:06, DMB*01:02, DOB*01:04, DPA1*01:03, DPB1*04:02, DQB1*03:518, DQB1*06:352, DQB1*06:395, DRA*01:02, DRA*01:05, DRA*01:06, DRA*01:07, DRA*01:08, DRB1*01:01, DRB1*01:02, DRB1*04:07, DRB1*04:334, DRB1*07:34, DRB1*11:321, DRB1*13:327, DRB1*15:04, DRB3*01:108, DRB3*02:01, DRB3*02:191, DRB3*02:25, E*01:13, A*02:01, B*07:02, B*08:01, B*51:01, B*52:01, C*07:01, C*12:02, DPB1*04:01, DQB1*05:01, DQB1*06:01, DRB1*01:03, DRB1*03:01, DRB1*15:02, DQB1*02:01, DRB1*04:01.

In some embodiments, the first set of HLA alleles associated with the risk of IBD comprises: DRA*01:05, A*02:01, DPB1*04:01, DOB*01:04, DRB3*02:25, B*51:01, DRB3*02:01, DMB*01:02, C*06:201, DQB1*05:01, E*01:13, DRB1*01:03, DRB1*15:04, C*02:205Q, DRB1*15:02, DQB1*06:352, DRA*01:07, DQB1*06:01, DRB1*04:334, DRB3*01:108, DRB1*11:321, DQB1*03:518, DRB1*01:02, DMA*01:06, DRB1*07:34, DRB3*02:191, B*52:01, C*12:02, DMA*01:05, DRA*01:08, DQB1*06:395, DRB1*13:327, and DRA*01:06.

In some embodiments, the second set of HLA alleles not associated with the risk of IBD comprises: DPA1*01:03, DMA*01:01, DRA*01:02, DPB1*04:02, B*07:02, DRB1*01:01, C*07:01, B*08:01, C*03:04, DRB1*04:01, DQB1*02:01, DRB1*03:01, and DRB1*04:07.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as an example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as an example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for predicting whether a subject will experience an immune-related adverse event (irAE) in response to administration of an immune checkpoint inhibitor (ICI) therapy to the subject, the method comprising:
using at least one processor to perform:
obtaining healthcare data for the subject, the healthcare data comprising at least two of: clinical data for the subject, RNA sequencing data for the subject, and immune receptor data for the subject;
determining, using at least some of the healthcare data, a likelihood that the subject will experience the irAE in response to administration of the ICI therapy, the determining comprising:
performing at least two of:
(a) processing the clinical data for the subject using a first machine learning (ML) model to output a first likelihood that the subject will experience the irAE in response to administration of the ICI therapy, wherein the first ML model is trained to predict, from clinical data for a particular subject, a likelihood that the particular subject will experience the irAE in response to administration of the ICI therapy,
(b) processing the RNA sequencing data for the subject using a second ML model to output a second likelihood that the subject will experience the irAE in response to administration of the ICI therapy, wherein the second ML model is trained to predict, from RNA sequencing data for a particular subject, a likelihood that the particular subject will experience the irAE in response to administration of the ICI therapy, and
(c) processing the immune receptor data for the subject using a third ML model to output a third likelihood that the subject will experience the irAE in response to administration of the ICI therapy, wherein the third ML model is trained to predict, from immune receptor data for a particular subject, a likelihood that the particular subject will experience the irAE in response to administration of the ICI therapy; and
processing two or more of the first, second, and third likelihoods using a fourth ML model trained to predict the likelihood that the subject will experience #14561833v1 the irAE in response to administration of the ICI therapy from one or more of the likelihoods that the subject will experience the irAE determined using two or more of first-, second- and third-ML models; and
outputting the likelihood that the subject will experience the irAE in response to administration of the ICI therapy.

2. The method of claim 1, further comprising:
outputting a recommendation to administer the ICI therapy to the subject when the likelihood that the subject will experience the irAE is less than or equal to a threshold.

3. The method of claim 2, further comprising:
administering the ICI therapy to the subject when the likelihood that the subject will experience the irAE is less than or equal to the threshold.

4. The method of claim 1, further comprising:
when the likelihood that the subject will experience the irAE is greater than or equal to a threshold:
generating, using the RNA sequencing data for the subject, human leukocyte antigen (HLA) input features indicative of HLA alleles present in a genome of the subject; and
processing the HLA input features using an ML model for predicting inflammatory bowel disease (IBD) to output a likelihood that the subject will develop IBD in response to the administration of the ICI therapy, wherein the ML model for predicting IBD is trained to predict, from HLA input features for a particular subject, a likelihood that the particular subject will develop IBD.

5. The method of claim 4, wherein generating the HLA input features using the RNA sequencing data for the subject comprises generating:
(i) a first input feature indicative of a number of HLA alleles present in the genome of the subject that are associated with a risk of IBD,
(ii) a second input feature indicative of a number of HLA alleles present in the genome of the subject that are not associated with the risk of IBD, and
(iii) one or more third input features, each of the one or more third input features indicative of a respective HLA allele present in the genome of the subject.

6. The method of claim 1,
wherein the healthcare data comprises the clinical data for the subject, and
wherein determining, using the at least some of the healthcare data, the likelihood that the subject will experience the irAE in response to administration of the ICI therapy comprises:
processing the clinical data for the subject using the first ML model to output the first likelihood that the subject will experience the irAE in response to administration of the ICI therapy, and
wherein the clinical data for the subject indicates: age, gender, diagnosis, disease stage, therapy type, and metastatic status for the subject.

7. The method of claim 1,
wherein the healthcare data comprises the RNA sequencing data for the subject, and
wherein determining, using the at least some of the healthcare data, the likelihood that the subject will experience the irAE in response to administration of the ICI therapy comprises:
processing the RNA sequencing data for the subject using the second ML model to output the second likelihood that the subject will experience the irAE in response to administration of the ICI therapy.

8. The method of claim 7, wherein processing the RNA sequencing data for the subject using the second ML model comprises:
determining a plurality of immune signatures using the RNA sequencing data for the subject, each of the plurality of immune signatures representing RNA expression levels for at least some genes in a respective gene group of a plurality of gene groups; and
processing the plurality of immune signatures using the second ML model to obtain the second likelihood that the subject will experience the irAE in response to administration of the ICI therapy.

9. The method of claim 8, wherein the RNA sequencing data for the subject indicates RNA expression levels for at least some genes in each group of at least some of the plurality of gene groups, the plurality of gene groups comprising:
LDHB glycolysis signature: LDHB, DGKA, GCNT4, TBC1D4, ETS1;
Treg and T-cell activation signature: ABCC1, ARID5B, BCL2, BIRC3, CCND2, CCR4, CD2, CD28, CISH, CTLA4, FAS, FOXP3, GATA3, ICOS, IL12RB2, IL2RA, IL2RB, LTA, MAF, MAP3K14, OPTN, P2RY10, PIM2, POU2AF1, RTKN2, SLAMF1, SOCS1, SOCS2, TIGIT, TRADD, TRAF1, TRAF2;
irAE-associated T-cell signature: TNFRSF4, CD28, KLRB1, TNFRSF18, CD40, IFNG, TRAT1, EOMES, CD69, CCR8, GZMA, TIGIT, TNFRSF9, ZAP70, TCF7, KLRK1, ICOS, CD8B, FASLG, CD27, IKZF2, PRF1, GZMB, LAIR2, GZMK, CCL5, CD5, GZMH, CD8A, PFKP, CD40LG, KLRD1, TBX21, NKG7, GNLY, CTLA4, TRAC;
Treg signature: FOXP3, CTLA4, IL2RA, CCR8, IKZF4, IKZF2, RTKN2, CCR4, FAS;
CD4-related signature: CD28, TCF7, IL2RA, CHMP7, CCR4, CAMK4, S1PR1, DUSP16, MAL, AQP3, CCR7, RASA3, CD40LG, GATA3, KCNA3, RCAN3, ZC3H12D, CD6, LRIG1, TRAF1, TRAT1, CD27, TRABD2A, TESPA1, ICOS, CACNA11, ITPKB, PIK3C2B, TNFRSF10A, CD5;
Antigen specific T-cell activation: TESPA1, SIRPG, CD3G, SLAMF6, CD27, LCK, IKZF3, FCMR, LDLRAP1, LTB, EPB41, LAT, CD3D, PTPRCAP, ADD3, CD2, MAP4K1, SIT1, ESYT1, UBASH3A, TRAF3IP3, CD3E, SAMD3, THEMIS, LIME1, LY9, GRAP, SKAP1, TCF7, ITM2A, KLRG1;
Hypoxia factors signature: FUT11, NDRG1, EPAS1, CA9, LDHA, LOX, SLC2A1, P4HA1, CA12, HK2, PDK1, PGK1, TPI1, ALDOA, PFKFB3;
LDHA glycolysis signature: HAVCR2, PGK1, LDHA, PSMA6, BPGM, PDIA3, PDIA6, PLIN2, SPPL2A, LGALS8, YARS, HSP90B1, MAGT1, SKIL, GSTO1;
Platelet signature: ITGA2B, ITGB3, SELP, MPL, GP1BA, GP1BB, TUBB1;
TNF signaling-associated signature: AREG, EREG, LAMB3, PLAU, PTX3;
Myeloid suppression signature: TGFB2, IL10, CCL24, CXCL8, S100A12, EBI3, MSR1, PTGS2, SLC11A1, TREM1, PLAUR;
M2 polarization signature: TGFB2, TGFB3, IL10, CCL18, IL33, CCL24; and
Autophagy signature ATG12, ATG9A, TFEB, RB1CC1, MAP1LC3B, GABARAPL2, ATG4B, ATG7, GABARAP, VMP1, ATG14, GABARAPL1, ATG13, NBR1.

10. The method of claim 8, wherein determining the plurality of immune signatures using the RNA sequencing data for the subject comprises:
determining gene group scores for respective gene groups in the at least some of the plurality of gene groups using the RNA expression levels.

11. The method of claim 8, wherein processing the RNA sequencing data for the subject using the second ML model comprises:
determining, using the RNA sequencing data for the subject, (i) a proportion of classical dendritic cells (cDCs) to dendritic cells and (ii) a proportion of memory T cells to T cells; and
processing (i) the proportion of cDCs to dendritic cells, (ii) the proportion of memory T cells to T cells, and (iii) the plurality of immune signatures, using the second ML model to output the second likelihood that the subject will experience the irAE in response to administration of the ICI therapy.

12. The method of claim 8, wherein the healthcare data further comprises immune cell data, and wherein processing the RNA sequencing data using the second ML model further comprises:
determining, using the immune cell data, (i) a proportion of classical dendritic cells (cDCs) to dendritic cells and (ii) a proportion of memory T cells to T cells; and
processing (i) the proportion of cDCs to dendritic cells, (ii) the proportion of memory T cells to T cells, and (iii) the plurality of immune signatures, using the second ML model to output the second likelihood that the subject will experience the irAE in response to administration of the ICI therapy.

13. The method of claim 1,
wherein the healthcare data comprises the immune receptor data for the subject, and
wherein determining, using the at least some of the healthcare data, the likelihood that the subject will experience the irAE in response to administration of the ICI therapy comprises:
processing the immune receptor data for the subject using the third ML model to output the third likelihood that the subject will experience the irAE in response to administration of the ICI therapy.

14. The method of claim 13, wherein the immune receptor data comprises B cell receptor sequence data and T cell receptor sequence data.

15. The method of claim 14, wherein processing the immune receptor data using the third ML model comprises:
determining, using the B cell receptor sequence data, a value indicative of B cell receptor diversity;
determining, using the T cell receptor sequence data, a value indicative of T cell receptor diversity;
determining, using the B cell receptor sequence data, a proportion of a number of IgH clonotypes having a particular variable gene with respect to a total number of IgH clonotypes; and
processing, using the third ML model, the value indicative of B cell receptor diversity, the value indicative of T cell receptor diversity, and the proportion of the number of IgH clonotypes associated with the particular variable gene with respect to the total number of IgH clonotypes.

16. The method of claim 15, wherein the value indicative of the B cell receptor diversity and the value indicative of the T cell receptor diversity are computed according to:
$$- \frac{1}{N} {\sum }_{n = 1}^{N} {\sum }_{i = 1}^{{s}_{N}} {p}_{i , N} ln ( {p}_{i , N} )$$
where:
N represents a number of receptor chains;
s_{N} represents a number of clonotypes for a particular receptor chain, and
p_{i,N} represents a proportion of a frequency of a particular clonotype with respect to a frequency of all clonotypes for the particular receptor chain.

17. The method of claim 15, wherein the particular variable gene is IgHV4-34.

18. The method of claim 1,
wherein the healthcare data comprises the clinical data for the subject, the RNA sequencing data for the subject, and the immune receptor data for the subject, and
wherein determining, using the at least some of the healthcare data, the likelihood that the subject will experience the irAE in response to administration of the ICI therapy comprises:
performing:
(a) processing the clinical data for the subject using the first ML model to output the first likelihood that the subject will experience the irAE in response to administration of the ICI therapy;
(b) processing the RNA sequencing data for the subject using the second ML model to output the second likelihood that the subject will experience the irAE in response to administration of the ICI therapy; and
(c) processing the immune receptor data for the subject using the third ML model to output the third likelihood that the subject will experience the irAE in response to administration of the ICI therapy.

19. A system, comprising:
at least one processor; and
at least one non-transitory computer-readable storage medium storing processor-executable instructions that, when executed by the at least one processor, cause the at least one processor to perform a method for predicting whether a subject will experience an immune-related adverse event (irAE) in response to administration of an immune checkpoint inhibitor (ICI) therapy to the subject, the method comprising:
obtaining healthcare data for the subject, the healthcare data comprising at least two of: clinical data for the subject, RNA sequencing data for the subject, and immune receptor data for the subject;
determining, using at least some of the healthcare data, a likelihood that the subject will experience the irAE in response to administration of the ICI therapy, the determining comprising:
performing at least two of:
(a) processing the clinical data for the subject using a first machine learning (ML) model to output a first likelihood that the subject will experience the irAE in response to administration of the ICI therapy, wherein the first ML model is trained to predict, from clinical data for a particular subject, a likelihood that the particular subject will experience the irAE in response to administration of the ICI therapy,
(b) processing the RNA sequencing data for the subject using a second ML model to output a second likelihood that the subject will experience the irAE in response to administration of the ICI therapy, wherein the second ML model is trained to predict, from RNA sequencing data for a particular subject, a likelihood that the particular subject will experience the irAE in response to administration of the ICI therapy, and
(c) processing the immune receptor data for the subject using a third ML model to output a third likelihood that the subject will experience the irAE in response to administration of the ICI therapy, wherein the third ML model is trained to predict, from immune receptor data for a particular subject, a likelihood that the particular subject will experience the irAE in response to administration of the ICI therapy; and
processing two or more of the first, second, and third likelihoods using a fourth ML model trained to predict the likelihood that the subject will experience the irAE in response to administration of the ICI therapy from one or more of the likelihoods that the subject will experience the irAE determined using two or more of first-, second- and third-ML models; and
outputting the likelihood that the subject will experience the irAE in response to administration of the ICI therapy.

20. At least one non-transitory computer-readable storage medium storing processor-executable instructions that, when executed by at least one processor, cause the at least one processor to perform a method for predicting whether a subject will experience an immune-related adverse event (irAE) in response to administration of an immune checkpoint inhibitor (ICI) therapy to the subject, the method comprising:
obtaining healthcare data for the subject, the healthcare data comprising at least two of: clinical data for the subject, RNA sequencing data for the subject, and immune receptor data for the subject;
determining, using at least some of the healthcare data, a likelihood that the subject will experience the irAE in response to administration of the ICI therapy, the determining comprising:
performing at least two of:
(a) processing the clinical data for the subject using a first machine learning (ML) model to output a first likelihood that the subject will experience the irAE in response to administration of the ICI therapy, wherein the first ML model is trained to predict, from clinical data for a particular subject, a likelihood that the particular subject will experience the irAE in response to administration of the ICI therapy,
(b) processing the RNA sequencing data for the subject using a second ML model to output a second likelihood that the subject will experience the irAE in response to administration of the ICI therapy, wherein the second ML model is trained to predict, from RNA sequencing data for a particular subject, a likelihood that the particular subject will experience the irAE in response to administration of the ICI therapy, and
(c) processing the immune receptor data for the subject using a third ML model to output a third likelihood that the subject will experience the irAE in response to administration of the ICI therapy, wherein the third ML model is trained to predict, from immune receptor data for a particular subject, a likelihood that the particular subject will experience the irAE in response to administration of the ICI therapy; and
processing two or more of the first, second, and third likelihoods using a fourth ML model trained to predict the likelihood that the subject will experience the irAE in response to administration of the ICI therapy from one or more of the likelihoods that the subject will experience the irAE determined using two or more of first-, second- and third-ML models; and
outputting the likelihood that the subject will experience the irAE in response to administration of the ICI therapy.
